(19) United States
(12) Patent Application Publication
(10) Pub. No.: US 2023/0119171 A1
(43) Pub. Date: Apr. 20, 2023

(54) BIOMARKER PANELS FOR STRATIFICATION OF RESPONSE TO IMMUNE CHECKPOINT BLOCKADE IN CANCER

(71) Applicant: CEDARS-SINAI MEDICAL CENTER, Los Angeles, CA (US)

(72) Inventors: Dan Theodorescu, Los Angeles, CA (US); Sungyong You, Los Angeles, CA (US); Keith Syson Chan, Los Angeles, CA (US)

(73) Assignee: CEDARS-SINAI MEDICAL CENTER, Los Angeles, CA (US)

(21) Appl. No.: 17/914,050

(22) PCT Filed: Mar. 31, 2021
(86) PCT No.: PCT/US2021/025204

§ 371 Date: Sep. 23, 2022

Related U.S. Application Data
(60) Provisional application No. 63/002,758, filed on Mar. 31, 2020.

(51) Int. Cl. C12Q 1/6886; A61K 45/00

(52) U.S. Cl. CPC C12Q 1/6886; A61K 45/05; C12Q 2600/106; C12Q 2600/158

(57) ABSTRACT

The present invention describes methods of detecting biomarkers in tumor tissue, as well as methods of improving responsiveness to or providing survival prognosis for a subject having received or in need of, an immune checkpoint inhibitor in the treatment of cancers such as bladder cancer, lung cancer, leukemia. Discoidin domain receptor (DDR)-driven gene signatures have been identified and validated to stratify patient response to anti-PD-L1 immune checkpoint therapy.

### CROSS-REFERENCE TO RELATED APPLICATIONS

This application includes a claim of priority under 35 U.S.C. § 119(e) to U.S. provisional patent application No. 63/002,758, filed Mar. 31, 2020, the entirety of which is hereby incorporated by reference.

### STATEMENT REGARDING FEDERALLY SPONSORED RESEARCH OR DEVELOPMENT

This invention was made with government support under Grant No. CA075115 awarded by National Institutes of Health. The government has certain rights in the invention.

### FIELD OF INVENTION

This invention relates to methods of detecting biomarkers in tumor tissue and uses thereof to direct the use of immune checkpoint inhibitors in cancer patients.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Antibody-based immune checkpoint therapy (ICT) are emerging as an important pillar of anti-cancer therapy, in addition to surgery, chemotherapy, radiation and targeted therapies. These ICTs function to unleash the cytotoxic activities of T-cells via targeting co-inhibitory receptors on T-cells, such as programmed cell death 1 (PD-1) and cytotoxic T lymphocyte antigen 4 (CTLA4), as well as PD-1 ligand 1 (PD-L1) on tumor cells or antigen presenting immune cells (i.e., myeloid cells). Immune checkpoint therapies have achieved remarkably durable clinical response in melanoma, non-small cell lung carcinoma (NSCLC), bladder cancer, and renal cell cancer (RCC). Despite these initial successes, more than 64-70% of patients do not respond to checkpoint blockade immunotherapy. For example, although immune checkpoint blockade (ICB) (e.g. anti-PD-L1 and anti-PD1) has received expedited approval for the treatment of advanced and metastatic bladder urothelial carcinomas (UCs) since 2017 and revolutionized the treatment landscape for bladder UCs, approximately 70-85% of patients with advanced urothelial cancer remain immune checkpoint non-responders, that is, they are considered non-responders to either anti-PD-1 or anti-PD-L1 antibodies.

There are no reliable predictive markers and gene-signatures currently available for the selection or stratification of responder patients. To distinguish ICT responders from non-responders, recent studies have employed different biomarkers, including PD-L1 overexpression (in epithelial cancer and immune cells), tumor mutational burden (TMB) and neoantigen load in tumor cells. In general, there are some associations of these biomarkers with ICT outcome. However, neither PD-L1 overexpression nor TMB as a single marker is sufficient to distinguish ICT responders from non-responders. A meta-analysis of 45 primary studies associated with ICT drug approvals by the US Food and Drug Administration (FDA) showed that PD-L1 overexpression (on cancer or immune cells) was predictive in only 28.9% of cases, while not predictive or not tested in the remaining 53.3% and 17.8% of cases respectively. For example, in bladder cancer which is a tumor type where a significant number of patients have responses, epithelial PD-L1 expression exhibited no association with ICT response, while there was an initial indication that PD-L1 expression on immune cells (i.e., myeloid cells) demonstrated association with ICT response. Meanwhile, increasing TMB has been associated with improved clinical outcome from ICT in various cancers, including bladder cancer. However, the variable detection methods (i.e., whole exome sequencing vs. targeted next-generation sequencing panels such as MSK-IMPACT) and the lack of universal TMB threshold values to predict ICT efficacy has made it difficult to use TMB as a single marker to predict ICT response in cancer patients. Thus, these previous studies point to the needs to identify more reliable biomarkers for stratifying ICT responders from non-responders.

There remains an unmet clinical need for a predictive signature that can reliably stratify immune checkpoint responders from non-responders, thereby matching responsive patient groups with ICB therapeutics can increase treatment efficacy. The present invention in various aspects address this need.

### SUMMARY OF INVENTION

Various embodiments provide for methods of selecting a cancer patient for administration of an immune checkpoint inhibitor by measuring the gene transcript expression level and/or the protein expression level of a population of signature genes from the tumor sample of the patient.

Various embodiments provide for methods of treating a subject with cancer by administering an immune checkpoint inhibitor (e.g., an anti-PD-L1 or anti-PD-1 therapy) to a subject who has been determined to have an expression pattern of a population of signature genes in a tumor sample of the subject.

Various embodiments provide for methods of detecting expression levels of signature genes in a biological sample of a subject, or analyzing a biological sample, by measuring the expression levels of signature genes in comparison to reference values.

Various embodiments provide for methods of determining if a cancer patient is predicted to respond to the administration of an immune checkpoint inhibitor by measuring the expression levels of signature genes in comparison to reference values in a cancer tissue of the subject.

Further embodiments provide for methods of determining risk associated with cancer in a cancer patient, comprising measuring expression levels of a set of signature genes in cancer cells of said cancer patient, thereby obtaining a risk score (RS) based on the expression levels of said set of signature genes, and determining risk of cancer for said cancer patient by comparing the risk score to a predefined risk score cut off threshold for said set of signature genes. In some embodiments, methods comprise measuring expression levels of a set of signature genes in cancer cells of the cancer patient, thereby obtaining a risk score (RS) based on the expression levels of said set of signature genes, wherein the cancer patient is determined to have an increased risk of poor survival if the RS is higher than a predefined RS cut off threshold, or wherein the cancer patient is determined to have a decreased risk of poor survival if the RS is lower than the predefined RS cut off threshold. In some embodiments, the predefined RS cut off threshold for a set of genes is a risk score exhibiting the lowest P value (to best separate between the groups) from hazard ratios (HR) of high-risk vs. low-risk groups versus the RS for the set of genes. In some embodiments, the predetermined RS cut off threshold is −0.77 for the 10 signature genes in Table 3 based on Z-score model; or the predetermined RS cut off threshold is −0.079 for the 19 signature genes in Table 4 based on Cox model; or the predetermined RS cut off threshold is 0.039 for the 4 signature genes in Table 5 base on Z-score model; or the predetermined RS cut off threshold is −0.059 for the 25 signature genes in Table 6 based on Cox model. In other embodiments, the predetermined RS cut off threshold is the median RS where there are an equal number of individuals in the high and low-risk groups.

In various embodiments, a population of signature genes include 1) a plurality of marker genes having substantial sequence identity with those set forth in Table 3; 2) a plurality of marker genes having substantial sequence identity with those set forth in Table 4; 3) a plurality of marker genes having substantial sequence identity with those set forth in Table 5; 4) a plurality of marker genes having substantial sequence identity with those set forth in Table 6; 5) polynucleotides which are complementary to any plurality of the marker genes in any of a)-d); 6) polypeptides encoded by any plurality of the marker genes in any of 1)-4); or 7) polypeptides have substantial sequence identity with those of 6).

In various embodiments, an expression pattern of the signature genes is: AQP3, NDUFA4L2, PALM, DHRS3, GGT5, GIPR, GALNT18, ANO1, PCDHGB2, LURAP1L, S100A2, GCNT3, CXCL6, MMP10, TFF1, ANXA10, FCGBP, IL33, TP53I11, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, and CRISP3 each having an expression level below respective reference value; and NEBL, MLIP, CSMD2, NXPH4, SCNN1B, IGFL1, DEFB1, IL13RA2, ALOX12, TMEM63C, CXCL2, WDR72, GUCY2C, B3GALT2, TRIM66, TPH1, S100A9, ODAPH, and NSUN7 each having an expression level above respective reference value.

In some embodiments, the cancer is bladder cancer. In some embodiments, the cancer is lung cancer, or nonsmall lung cancer. In some embodiments, the cancer is leukemia. In some embodiments, the cancer is urothelial bladder cancer.

Screening methods for candidate agents to improve cancer treatments with immune checkpoint blockade therapies, as well as assay systems in relation thereto, are also provided.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-IC depict an inverse relationship of DDR1 and DDR2 mRNA expression and enriched cellular processes in the TCGA bladder cancer patients with TNM stage T2 and higher. FIG. 1A is a scatter plot with a regression line showing inverse relationship between the mRNA expression levels of DDR1 and DDR2 from 259 specimens in Example 1. FIG. 1B is a heat map depicting mRNA expression pattern of DDR1 and DDR2. FIG. 1C is a heat map displaying gene set enrichment analysis (GSEA) analysis results of enriched hallmark gene sets in DDR2 expression high tumors compared to DDR2 low tumors.

FIGS. 2A-2C depict the DDR1 and DDR2 signatures as surrogate marker for measuring DDR1 and DDR2 activation status. FIG. 2A depicts generation of tumors with enforced expression of DDR1 or control based on T24 human bladder cancer (BC) cells. FIG. 2B depicts generation of tumors/cells treated with shDDR2 or control scrambles. These models are based on NA13 mouse BC tumor. FIG. 2C depicts a workflow of selecting the DDR1 and DDR2 gene expression signature from the RNA-seq data.

FIGS. 3A-3D depict evaluation of Top50 genes from DDR1 and DDR2 data. FIG. 3A is a bar chart displaying the average fold change of the distinct combination of the top genes from DDR1 in vivo data in both DDR1 in vivo and IMvigor data. FIG. 3B shows bar charts displaying average fold change of the top 50 genes from DDR2 in vitro and in vivo data in both DDR2 in vitro (or in vivo) and IMvigor data. FIG. 3C is a heat map depicting enrichment result of the 16 hallmark gene sets by DDR1 and DDR2 expression and activation status. DDR1/2 activation status was computed by the weighted Z-score method and used to stratified tumors into two groups with high and low activation status of DDR1 or DDR2 in IMvigor cohort. FIG. 3D is a box plot depicting DDR gene signature score against the Complete Responder group and Progressive Disease group from IMvigor cohort.

FIG. 4 depicts clinical association of DDR1 and DDR2 activation status in TCGA BC cohort (N=259; T2+). Fitted line in the diagrams on the left depicts a trend of Hazard Ratio (HR) versus DDR1 or DDR2 activation scores in TCGA BC data. Star marker with solid vertical line indicates estimated HR at the optimal point of DDR1 or DDR2 activation score showing the best separation of BC specific mortality in TCGA BC cohort. On the right, Kaplan-Meier curves shows the survival rate of the two groups stratified at the optimal point of DDR1 or DDR2 activation in TCGA BC cohort.

FIG. 5 depicts clinical association of DDR1 activation in IMvigor study. Kaplan-Meier curves shows survival rate difference between DDR1 activation high and low groups. Three different stratifications were applied to examine the survival separation patterns. Fitted line is depicting trend of HR versus DDR1 activation scores in IMvigor data. Star marker with solid vertical line indicates estimated HR at the optimal point of DDR1 activation score showing the best separation of overall survival in IMvigor cohort.

FIG. 6 depicts clinical association of DDR2 activation in IMvigor study. Kaplan-Meier curves shows survival rate difference between DDR2 activation high and low groups. Three different stratifications were applied to examine the survival separation patterns. Fitted line is depicting trend of HR versus DDR2 activation scores in IMvigor data. Star marker with solid vertical line indicates estimated HR at the optimal point of DDR2 activation score showing the best separation of overall survival in IMvigor cohort.

FIG. 7 depicts a schematic view of gene selection process for model construction. Given the up- or down-regulated genes from DDR1 and DDR2 RNA-seq data, two different subtractive approaches were employed and made two different gene models based on the two genes signatures for DDR1 and DDR2, respectively, which are corresponding to Z-score model and Cox model. OS=Overall Survival; CR=Complete Response; PD=Progressive Disease.

FIG. 8 depicts clinical association of DDR1 Risk Score (RS) in IMvigor study. Upper and lower panels show survival analysis results for 10-gene DDR1 Z-score model and 19-gene DDR1 Cox Risk Score model, respectively. Fitted line is depicting trend of HR versus DDR1 RS in IMvigor data. Star marker with solid pink line indicates estimated HR at the optimal point of DDR1 RS showing the best separation of overall survival in IMvigor cohort. Kaplan-Meier curves shows survival rate difference between DDR1 RS high and low groups.

FIG. 9 depicts clinical association of DDR2 RS in IMvigor study. Upper and lower panels show survival analysis results for 4-gene DDR2 Z-score model and 25-gene DDR2 Cox Risk Score model, respectively. Fitted line is depicting trend of HR versus DDR2 RS in IMvigor data. Star marker with solid pink line indicates estimated HR at the optimal point of DDR2 RS showing the best separation of overall survival in IMvigor cohort. Kaplan-Meier curves shows survival rate difference between DDR2 RS high and low groups.

FIG. 10 depicts clinical association of DDR RS in IMvigor study. Upper and lower panels show survival analysis results for 14-gene DDR Z-score model and 44-gene DDR Cox Risk Score model, respectively. Fitted line is depicting trend of HR versus DDR RS in IMvigor data. Star marker with solid vertical line indicates estimated HR at the optimal point of DDR RS showing the best separation of overall survival in IMvigor cohort. Kaplan-Meier curves shows survival rate difference between DDR RS high and low groups.

FIGS. 11A-11F depict the expression of DDR genes in human bladder tumors as a function of molecular subtype and clinical outcome. FIG. 11A. Heatmap depicts expression pattern of DDR1 and DDR2 in bladder TCGA cohort described in Example 1.9 Materials and Methods (n=259). FIG. 11B. Scatter plot with a regression line shows inverse relationship between DDR1 and DDR2 expression. Hierarchical clustering was performed using Manhattan distance and ward linkage method. FIG. 11C. Stacked bar graph depicts distribution of the tumors from bladder TCGA cohort by bladder cancer consensus subtypes. Tumor samples in each subtype were stratified into three groups by DDR expression at tercile values. FIGS. 11D and 11E. Box plot shows DDR1 (11D) and DDR2 (11E) expression in bladder cancer consensus subtypes. FIG. 11F. Kaplan-Meier survival curves for DDR1 expression (upper) and DDR2 expression (lower) in bladder TCGA cohort. Tumors were stratified into high and low groups at median expression of DDR. Significance of differential survival between the groups were tested by Log-Rank test. HR: Hazard rate.

FIGS. 12A-12I depict the impact of DDR expression on gene set enrichment analysis (GSEA), molecularly defined cellular composition and T cell inflamed GEP in human bladder tumors. FIG. 12A. Heatmap displays differentially enriched hallmark gene sets between DDR1^{high} and DDR2^{high} bladder tumors. FIG. 12B. Bar graphs depict Spearman's correlation coefficient of DDR1/2 expression and 23 immune infiltration scores described in Example 1.9 Materials and Methods. FIGS. 12C and 12E. TCGA Pan-cancer (n=10,323) analysis of DDR1/2 expression and T cell inflamed GEP score shows negative correlation with DDR1 expression (12C) and positive correlation with DDR2 expression (12E). Spearman's method was used to estimate correlation coefficient, with a regression line indicated in each plot. FIGS. 12D and 12F. Scatter plots and regression lines shows relationship of DDR1 (12D) and DDR2 (12F) expression with GEP score in bladder TCGA tumors (n=259). FIG. 12G. Tumors were stratified by T cell inflamed GEP score at the median of bladder TCGA cohort (n=259). FIG. 12H. Kaplan-Meier curves shows survival patterns of the four group by DDR1 expression and T cell inflamed GEP score. Multiple Log-Rank tests were performed with DDR1^{low} & GEP^{low} group as a base line. FIG. 12I. Kaplan-Meier curves shows survival patterns of the four group by DDR2 and T cell inflamed GEP score. HR: Hazard rate.

FIGS. 13A-13J depict the association of DDR expression with TME features and immune checkpoint therapy response in human bladder cancer. FIG. 13A. Bar graph depicts Spearman's correlation coefficient of DDR1 and DDR2 expression and immune cell type scores (n=23). FIG. 13B. Heatmap and scatter plot shows inverse relationship of DDR1 and DDR2 expression in IMvigor210 cohort. FIGS. 13C and 13D. Box plots depict expression distribution of DDR1 by immunotherapy response groups in IMvigor210 cohort (n=298). FIGS. 13E and 13F. Box plots depict expression distribution of DDR2 by immunotherapy response groups in IMvigor210 cohort (n=298). FIGS. 13G and 13H. Kaplan-Meier survival curves for DDR1 (13G) and DDR2 (13H) expression in IMvigor210 cohort. Tumors were stratified into high and low groups at median expression of DDR. Significance of differential survival between the groups were tested by Log-Rank test. FIG. 13I. Kaplan-Meier curves shows survival patterns of the four group by DDR1 expression and T cell inflamed GEP score. Multiple Log-Rank tests were performed with DDR1^{low} & GEP^{low} group as a base line. FIG. 13J. Kaplan-Meier curves shows survival patterns of the four group by DDR2 and T cell inflamed GEP score. Multiple Log-Rank tests were performed with DDR2^{low} & GEP^{high} group as a base line. Table 11 shows hazard ratio (HR), significance level (P-value) and confidence interval (CI) for each comparison.

FIGS. 14A-14F depict differentially expressed genes in response to DDR1 and DDR2 expression changes in mouse bladder cancer models. FIG. 14A. Generation of tumors with enforced expression of DDR1 and control based on T24 human bladder tumor cells, and NA13 mouse bladder tumor treated with shDDR2 and control scrambles. FIGS. 14B and 14C. Volcano plots depict differential expression of the genes perturbed by DDR1 (14B) or DDR2 (14C) in murine models. With enforced expression of DDR1, some genes have an elevated expression level including IL33, TFF1, ANXA10, FCGBP, and LINC02615; and some genes have a decreased level of expression, including PDPN, LINC02154, RYR2, MAGEC2, and AGMO. With knockdown of DDR2, some genes have an elevated expression level including Atp2a1, Ckm, Pvalb, Pygm, and Pdk4; and some genes have a decreased level of expression, including Chi14, Alas2, Hba-a1, Retnia, and Grp. FIG. 14D. Bar chart shows uniquely enriched hallmark gene sets by the up-regulated genes by DDR1 overexpression. FIG. 14E. Bar chart shows uniquely enriched hallmark gene sets by the down-regulated genes by shDDR2. FIG. 14F. Common and differentially enriched hallmark gene sets by up genes by DDR1 overexpression and down genes by shDDR2.

FIG. 15 depicts development and characterization of DDR gene expression signatures and their scores. A flow chart shows selection process of the differentially expressed genes perturbed by DDR1 or DDR2 in models, followed by functional evaluation process of the top 50 genes from DDR1 or DDR2 model through pathway analysis and survival analysis.

FIGS. 16A-16E depict the evaluation of DDR gene signature scores in bladder cancer patients treated with immune checkpoint therapy. FIGS. 16A and 16B. Bar plots depict enrichment KEGG pathways by DDR1 (16B) or DDR2 (16A) active tumors. DDR1/2 gene expression score was computed by the weighted Z-score method and used to stratify tumors into two groups with high and low DDR1 or DDR2 scores. Pathways were sorted by its DDR score and selected 10 pathways from the top of the list. FIG. 16C. Stacked bar graph depicts distribution of the tumors from IMvigor210 cohort by bladder cancer consensus subtypes. Tumor samples in each subtype were stratified into three groups by DDR score at tercile values. FIGS. 16D and 16E. Box plot shows DDR1 (16D) and DDR2 (16E) scores in bladder cancer consensus subtypes in IMvigor210 cohort.

FIGS. 17A-17D depict the association of DDR gene signature scores with immune checkpoint therapy response of bladder cancer patients. FIGS. 17A and 17B. Boxplots depicts DDR1 (17A) and DDR2 (17B) scores against the CR group and PD group from IMvigor210 cohort. FIGS. 17C and 17D. Kaplan-Meier survival curves for DDR1 (17C) and DDR2 (17D) score in IMvigor210 cohort. Tumors were stratified into high and low groups at median score level of IMvigor210 cohort. Significance of differential survival between the groups were tested by Log-Rank test.

FIGS. 18A-18I depict the development and validation of DDR gene signatures for predicting immunotherapy response. Given the up- or down-regulated genes from DDR murine models, two branches subtractive approaches were employed and made two different gene models based on the two genes signatures for DDR1 and DDR2, respectively, which are corresponding to Z-score model and Cox model. FIGS. 18A and 18B. Clinical association of DDR1 gene models. FIG. 18A and FIG. 18B show survival analysis results for 10-gene DDR1 Z-score model (CS-10) and 19-gene DDR1 Cox Risk Score model (CS-19), respectively. FIG. 18C. Clinical association of DDR2 gene models. Left and right panels show survival analysis results for 4-gene DDR2 Z-score model (CS-4) and 25-gene DDR2 Cox Risk Score model (CS-25), respectively. FIG. 18D. Survival curves of CS-10 high and low groups in LumP and Ba/Sq subtypes in IMvigor210 cohort.

FIG. 18E. Survival curves of CS-19 high and low groups in LumP and Ba/Sq subtypes in IMvigor210 cohort. FIG. 18F. Survival curves of CS-4 high and low groups in Stroma-rich subtype in IMvigor210 cohort. FIG. 18G. Survival curves of CS-25 high and low groups in Stroma-rich subtype in IMvigor210 cohort. FIG. 18H. Survival curves of CS-4 high and low groups in non-small cell lung cancer (NSCLC) cohort treated with anti PD-L1 therapy from Tempus. FIG. 18I. Survival curves of CS-25 high and low groups in NSCLC cohort treated with anti PD-L1 therapy from Caris.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed, Cold Spring Harbor Laboratory Press (Cold Spring Harbor, N.Y. 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor N.Y., 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The TNM system is a classification system to stage different types of cancer based on certain standards. Staging is the process of finding out how much cancer is in a person's body and where it's located. It's how the doctor determines the stage of a person's cancer. In the TNM system, each cancer is assigned a letter or number to describe the tumor (T), node (N), and metastases (M). T stands for the original (primary) tumor; N tells whether the cancer has spread to the nearby lymph nodes; and M tells whether the cancer has spread to distant parts of the body. Numbers after the T (such as T1, T2, T3, and T4) might describe the tumor size and/or amount of spread into nearby structures. The higher the T number, the larger the tumor and/or the more it has grown into nearby tissues. For example, TX means the tumor can't be measured; TO means there is no evidence of a primary tumor (it cannot be found); and Tis means that the cancer cells are only growing in the most superficial layer of tissue, without growing into deeper tissues, which may also be called in situ cancer or pre-cancer. Numbers after the N (such as N1, N2, and N3) might describe the size, location, and/or the number of nearby lymph nodes affected by cancer. The higher the N number, the greater the cancer spread to nearby lymph nodes. For example, NX means the nearby lymph nodes cannot be evaluated; and NO means nearby lymph nodes do not contain cancer. Similarly, M0 means that no distant cancer spread was found; while M1 means that the cancer has spread to distant organs or tissues (distant metastases were found).

“Stage grouping”: Once the values for T, N, and M have been determined, they are combined to assign an overall stage. For most cancers, the stage is a Roman numeral from I to IV, where stage IV (4) is the highest and means the cancer is more advanced than in the lower stages. Sometimes stages are subdivided as well, using letters such as A and B. Stage 0 is carcinoma in situ for most cancers. This means the cancer is at a very early stage, is only in the area where it first developed, and has not spread. Not all cancers have a stage 0. Stage I cancers are the next least advanced and often have a good prognosis (outlook). The outlook is usually not as good for higher stages. Typically, staging is done when a person is first diagnosed, before any treatment is given.

A “cancer” or “tumor” as used herein refers to an uncontrolled growth of cells which interferes with the normal functioning of the bodily organs and systems, and/or all neoplastic cell growth and proliferation, whether malignant or benign, and all pre-cancerous and cancerous cells and tissues. Included in this definition are benign and malignant cancers, as well as dormant tumors or micro-metastases. The term “invasive” refers to the ability to infiltrate and destroy surrounding tissue. Examples of cancer include, but are not limited to bladder cancer, B-cell lymphomas (Hodgkin lymphomas and/or non-Hodgkin lymphomas), brain tumor, urothelial cancer, breast cancer, colon cancer, lung cancer, hepatocellular cancer, gastric cancer, pancreatic cancer, cervical cancer, ovarian cancer, liver cancer, cancer of the urinary tract, thyroid cancer, renal cancer, carcinoma, melanoma, head and neck cancer, brain cancer, and prostate cancer. In further embodiments, “pan-cancer” cohort analysis refers to analysis across a plurality of tumor types from the Pan-Cancer Analysis of Whole Genomes (PCAWG) Consortium of the International Cancer Genome Consortium (ICGC) and The Cancer Genome Atlas (TCGA), including 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or all 38 tumor types selected from the group of glioblastoma (CNS-GBM), medulloblastoma and variants (CNS-Medullo), oligodendroglioma (CNS-Oligo), pilocytic astrocytoma (CNS-PiloAstro), malignant melanoma (Skin-Melanoma), papillary cholangiocarcinoma (Billary-AdenoCA), transitional cell carcinoma (Bladder-TCC), colon/rectum adenocarcinoma (ColoRect-AdenoCA), oesophagus adenocarcinoma (Eso-AdenoCA), hepatocellular carcinoma (Liver-HCC), lung adenocarcinoma (Lung-AdenoCA), lung squamous cell carcinoma (Lung-SCC), pancreas adeno. Acinar cell Ca. (Panc-AdenoCA), pancreas neuroendocrine carcinoma (Panc-Endocrine), prostate adenocarcinoma (Prost-AdenoCA), stomach adenocarcinoma (Stomach-AdenoCA), thyroid adenocarcinoma (Thy-AdenoCA), osteoblastoma or osteofibrous dysplasia (Bone-Benign), chondroblastoma or chrondromyxoid fibroma (Bone-Benign), adamantinoma or chordoma (Bone-Epith), osteosarcoma (Bone-Osteosarc), leiomyosarcoma (SoftTissue-Leiomyo), liposarcoma (SoftTissue-Liposarc), cervix adenocarcinoma (Cervix-AdenoCA), cervix squamous cell carcinoma (Cervix-SCC), head/neck squamous cell carcinoma (Head-SCC), kidney adenocarcinoma chromophobe type (Kidney-ChRCC), kidney clear cell adenocarcinoma papillary type (Kidney-RCC), lymphoid Burkitt or diffuse large B-cell or follicular or marginal (Lymph-BNHL), chronic lymphocytic leukaemia (Lymph-CLL), acute myeloid leukaemia (Myeloid-AML), myelodysplastic syndrome (Myeloid-MDS), myeloproliferative neoplasm (Myeloid-MPN), ovary adenocarcinoma (Ovary-AdenoCA), uterus adeno. endometrioid (Uterus-AdenoCA), breast infiltrating duct carcinoma (Breast-AdenoCA), breast duct micropapillary carcinoma (Breast-DCIS), and breast lobular carcinoma (Breast-LobularCA).

The markers of the invention are useful for predicting outcome of immune checkpoint therapies in multiple cancer types, including without limitation, bladder cancer, lung cancer (e.g., non-small cell lung cancer), head and neck cancer, glioma, gliosarcoma, anaplastic astrocytoma, medulloblastoma, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullary carcinoma, mastocytoma, mesotheliorma, synovioma, melanoma, leiomyosarcoma, rhabdomyosarcoma, neuroblastoma, retinoblastoma, oligodentroglioma, acoustic neuroma, hemangioblastoma, meningioma, pinealoma, ependymoma, craniopharyngioma, epithelial carcinoma, embryonal carcinoma, squamous cell carcinoma, base cell carcinoma, fibrosarcoma, myxoma, myxosarcoma, liposarcorna, chondrosarcoma, osteogenic sarcoma, and leukemia. In some embodiments the cancer may be bladder cancer, lung cancer or head and neck cancer.

“Discoidin domain receptors” (DDRs) are receptor tyrosine kinases (RTKs) that recognize fibrillar collagens. RTKs are a class of high-affinity cell surface receptors. Unlike other RTK, DDRs' ligands are solid extracellular matrix components that are abundantly present in the pericellular environment. DDRs are type I transmembrane proteins. Their domain structure consists of a collagen binding discoidin (DS) domain, a DS-like domain, an extracellular juxtamembrane (JM) region, a transmembrane region, an intracellular JM region and a tyrosine kinase (TK) domain.

A subject can be one who has been previously diagnosed with or identified as suffering from or having a disease-state in need of monitoring (e.g., cancer or infectious disease) or one or more complications related to such a disease-state, and optionally, have already undergone treatment for the disease-state or the one or more complications related to the disease/condition. Alternatively, a subject can also be one who has not been previously diagnosed as having a disease-state or one or more complications related to the disease/condition. For example, a subject can be one who exhibits one or more risk factors for a disease-state or one or more complications related to a disease-state or a subject who does not exhibit risk factors. A “subject in need” of treatment for a particular disease-state can be a subject having that disease/condition, diagnosed as having that condition, or at risk of developing that disease. The terms, “patient”, “individual” and “subject” are used interchangeably herein. A “subject” means a human or animal. Usually the animal is a vertebrate such as a primate, rodent, domestic animal or game animal. In various embodiment, the subject is a human in the methods. In some embodiments, the subject is a human having bladder cancer. In further embodiments, the subject is a human having bladder cancer who does not have a urothelial cancer.

The terms “control,” “control subject,” “normal,” “normal control subject,” and “a subject who does not have cancer” are intended to refer to a subject who has not been diagnosed with cancer, or who is cancer-free as a result of surgery to remove the diseased tissue. A non-cancer subject may be healthy and have no other disease, or they may have a disease other than cancer.

A biological marker (“biomarker” or “marker”) is a characteristic that is objectively measured and evaluated as an indicator of biologic processes, pathogenic processes, or pharmacological responses to therapeutic interventions, consistent with NIH Biomarker Definitions Working Group (1998). Markers can also include patterns or ensembles of characteristics indicative of particular biological processes. The biomarker measurement can increase or decrease to indicate a particular biological event or process. In addition, if the biomarker measurement typically changes in the absence of a particular biological process, a constant measurement can indicate occurrence of that process. A plurality of biomarkers includes at least two or more biomarkers (e.g., at least 2, 3, 4, 5, 6, and so on, in whole integer increments, up to all of the possible biomarkers) identified by the present invention, and includes any combination of such biomarkers. In various embodiments, such biomarkers are selected from any of the markers listed in the Table 3, Table 4, Table 5, Table 6, Table 7, or Table 8. In one embodiment, the plurality of biomarkers used in the present invention includes all of the biomarkers listed in Table 3. In one embodiment, the plurality of biomarkers used in the present invention includes all of the biomarkers listed in Table 4. In one embodiment, the plurality of biomarkers used in the present invention includes all of the biomarkers listed in Table 5. In one embodiment, the plurality of biomarkers used in the present invention includes all of the biomarkers listed in Table 6. In one embodiment, the plurality of biomarkers used in the present invention includes all of the biomarkers listed in Table 7. In one embodiment, the plurality of biomarkers used in the present invention includes all of the biomarkers listed in Table 8. In one embodiment, the plurality of biomarkers used in the present invention includes all of the biomarkers listed in any two, three, four, five, or all six of Tables 3-8.

The term “expression levels” refers to a quantity reflected in or derivable from the gene or protein expression data, whether the data is directed to gene transcript accumulation or protein accumulation or protein synthesis rates, etc. In some embodiments, the term “expression level” refers to the amount of gene transcript accumulation; and in some embodiments, the term “expression level” refers to the amount of protein accumulation; and in other embodiments, the term “expression level” refers to the amount of either gene transcript accumulation or protein transcript accumulation. The phrase “gene expression” or “protein expression” includes any information pertaining to the amount of gene transcript or protein present in a sample, as well as information about the rate at which genes or proteins are produced or are accumulating or being degraded (e.g., reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information.” Such information may be given in the form of amounts per cell, amounts relative to a control gene or protein, in unitless measures, etc.; the term “information” is not to be limited to any particular means of representation and is intended to mean any representation that provides relevant information.

The gene markers identified in any of Tables 3-8 may be polynucleotides that are genomic DNA, cDNA, or mRNA transcripts. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example, a polynucleotide includes a gene, a gene fragment, cDNA, isolated DNA, mRNA, tRNA, rRNA, isolated RNA of any sequence, recombinant polynucleotides, primers, probes, plasmids, and vectors. Additionally, the invention provides polynucleotides that have substantial sequence similarity to a polynucleotide that is described in any of Tables 3-8. Two polynucleotides have “substantial sequence identity” when there is at least 80% sequence identity, at least 90% sequence identity, at least 95% sequence identity or at least 99% sequence identity between their amino acid sequences or when the polynucleotides are capable of forming a stable duplex with each other under stringent hybridization conditions. In some embodiments, the invention provides polynucleotides that have at least 95% sequence identity to a polynucleotide described in any of Tables 3-8.

Polypeptides encoded by the gene markers identified in any of Tables 3-8 may reflect a single polypeptide appearing in a database. In general, the polypeptide is the largest polypeptide found in the database. But such a selection is not meant to limit the polypeptide to those corresponding to those single polypeptides. Accordingly, in another embodiment, the invention provides a polypeptide that is a fragment, or a homolog or allele of a marker described in any of Tables 3-8. Additionally, the present invention includes polypeptides that have substantially similar sequence identity to the polypeptides encoded by the gene markers in any of Tables 3-8, or when polynucleotides encoding the polypeptides are capable of forming a stable duplex with each other under stringent hybridization conditions. For example, conservative amino acid substitutions may be made in polypeptides to provide functionally equivalent variants of the foregoing polypeptides, i.e., the variants retain the functional capabilities of the polypeptides. As used herein, a “conservative amino acid substitution” refers to an amino acid substitution that does not alter the relative charge or size characteristics of the protein in which the amino acid substitution is made. “Substantially sequence identity” refers to at least 80% sequence identity, at least 90% sequence identity, at least 95% sequence identity, at least 96% sequence identity, at least 97% sequence identity, at least 98% sequence identity, or at least 99% sequence identity to those polypeptides encoded by the gene markers identified in any of Tables 3-8.

Generally, a “fragment” of a polypeptide refers to a plurality of amino acid residues comprising an amino acid sequence that has at least 5 contiguous amino acid residues, at least 10 contiguous amino acid residues, at least 20 contiguous amino acid residues or at least 30 contiguous amino acid residues of a sequence of the polypeptide. A “fragment” of polynucleotide refers to a polymer of nucleic acid residues comprising a nucleic acid sequence that has at least 15 contiguous nucleic acid residues, at least 30 contiguous nucleic acid residues, at least 60 contiguous nucleic acid residues, or at least 90%, at least 91%, at least 92%, at least 93%, at least 94%, at least 95%, at least 96%, at least 97%, at least 98%, or at least 99%, of a sequence of the polynucleotide. In some embodiment, the fragment is an antigenic fragment, and the size of the fragment will depend upon factors such as whether the epitope recognized by an antibody is a linear epitope or a conformational epitope. Thus, some antigenic fragments will consist of longer segments while others will consist of shorter segments, (e.g. 5, 6, 7, 8, 9, 10, 11 or 12 or more amino acids long, including each integer up to the full length of the polypeptide). Those skilled in the art are well versed in methods for selecting antigenic fragments of proteins.

Homologs and alleles of the polypeptide markers of the invention can be identified by conventional techniques. A homolog to a polypeptide is a polypeptide from a human or other animal that has a high degree of structural similarity to the identified polypeptides. Identification of human and other organism homologs of polypeptide markers identified herein will be familiar to those of skill in the art. In general, nucleic acid hybridization is a suitable method for identification of homologous sequences of another species (e.g., human, cow, sheep), which correspond to a known sequence. Standard nucleic acid hybridization procedures can be used to identify related nucleic acid sequences of selected percent identity. For example, one can construct a library of cDNAs reverse transcribed from the mRNA of a selected tissue (e.g., bladder) and use the nucleic acids that encode polypeptides identified herein to screen the library for related nucleotide sequences. The screening preferably is performed using high-stringency conditions (described elsewhere herein) to identify those sequences that are closely related by sequence identity. Nucleic acids so identified can be translated into polypeptides and the polypeptides can be tested for activity.

The markers may be detected by a method known to those of skill in the art. In one embodiment, the expression of the marker genes is detected by detecting the presence of transcripts of the gene in cells in a biological sample. The expression of the marker genes may be detected by detecting hybridization of at least a portion of the gene or a transcript thereof, to a nucleic acid molecule comprising a portion of the gene and a transcript thereof in a nucleic acid array. The expression of the marker genes may also be detected by obtaining RNA from the cancer tissue sample; generating cDNA from the RNA; amplifying the cDNA with probes or primers for marker genes; and obtaining from the amplified cDNA the expression levels of the genes or gene expression products in the sample. Detection of the presence or number of copies of all or a part of a marker gene of the invention may be performed using techniques such as Southern analysis, in which total DNA from a cell or tissue sample is extracted, is hybridized with a labeled probe (e.g., a complementary DNA molecule), and the probe is detected; or techniques such as direct sequencing, gel electrophoresis, column chromatography, and quantitative PCR. In another embodiment, the protein expression of markers may be detected by mass spectrometry, chromatographic separations, 2-D gel separations, binding assays (e.g., immunoassays, ELISA), competitive inhibition assays, and so on, or a combination thereof.

The present invention also encompasses reagents or molecules which specifically bind the markers. The term “specifically binding,” refers to the interaction between binding pairs (e.g., an antibody and an antigen or aptamer and its target). In some embodiments, the interaction has an affinity constant of at most 106 moles/liter, at most 10-?moles/liter, or at most 10-8 moles/liter. In other embodiments, the phrase “specifically binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

In various embodiments, the level of the markers is compared to a standard level or a reference level. Typically, the standard biomarker level or reference range is obtained by measuring the same marker or markers in a set of normal controls. Measurement of the standard biomarker level or reference range need not be made contemporaneously; it may be a historical measurement. Preferably the normal control is matched to the patient with respect to some attribute(s) (e.g., age). Depending upon the difference between the measured and standard level or reference range, the patient can be diagnosed as predicted to respond to the radiation therapy or as not predicted to respond to the anti-PD-1 or anti-PD-L1 based immune checkpoint therapy. The markers of this invention may be used for diagnostic and prognostic purposes, as well as for therapeutic, drug screening and patient stratification purposes (e.g., to group patients into a number of “subsets” for evaluation), as well as other purposes described herein. The markers of the invention are useful in methods for monitoring progression of cancer and/or response to therapy. The markers are also useful in methods for treating cancer and for evaluating the efficacy of treatment for the disease. Such methods can be performed in human and non-human subjects. The markers may also be used as pharmaceutical compositions or in kits. The markers may also be used to screen candidate compounds that modulate their expression. The markers may also be used to screen candidate drugs for treatment of cancer. Such screening methods can be performed in human and non-human subjects. We previously have shown that a higher immune cell expression of PD-L1 (>5% staining, compared to <5% staining) is associated with a lower progression disease and a higher complete response, and that a gene set associated with CD8+T-effector (Teff) cells (CD8+ Teff-signature score) is positively associated with response and overall survival. We recently identified discoidin domain receptor 2 (DDR2) from an in vivo synthetic lethality screen as a target for enhancing ICT response; and further validated the functional depletion and pharmaceutical blockade of DDR2 (e.g., with dasatinib) as a viable approach to sensitize ICT response in preclinical models of bladder, breast, colon, sarcoma, and melanoma. Therefore, we reasoned that the evaluation of downstream genes that are modulated by DDR2 and/or its closely family member DDR1, when formulated as “gene signature scores” might be able to stratify patient response to ICT. Here we show that this indeed is possible and such signatures can stratify response to anti PD-L1 therapy in several patient cohorts in different tumor types. We provide a predictive signature that can reliably stratify immune checkpoint responders from non-responders.

Discoidin domain receptor DDR1 and DDR2, members of a collagen receptor family, have been identified as contributors to bladder cancer metastasis. Our findings reveal DDR1 expression is associated with low T cell infiltration and high tumor-associated neutrophils (TANs)—an immune desert phenotype (i.e., a lack of an immune response present in the tumor; no T cell army present to attack the tumor); while that of DDR2 shows an inflamed phenotype infiltrated with T cells and M2 macrophages with a TGF-β-driven activated stroma (i.e., inflamed tumor has an army of T Cells ready to attack the cancer from inside—active immune response within the tumor, but there may still be inhibitory factors preventing the active immune response from actually destroying all of the cancer cells). As such, identification of a gene signature based on DDR1 and DDR2 transcriptional biology is a useful predictor of response to ICB.

Various embodiments the present invention are based, at least in part, on these findings. The sets of gene markers of the invention are set forth in each of Tables 3-8, and are identified by the gene symbol, gene name and the log 2FC (fold change) between complete responder (CR) and progressive disease (PD) detailed in Examples 2-5. The polynucleotide sequences of these genes, as well as the sequences of the polypeptides encoded by them are publicly available and known to one having average skill in the art. All information associated with the publicly available identifiers and accession numbers, including the nucleic acid sequences of the associated genes and the amino acid sequences of the encoded proteins is incorporated herein by reference in its entirety. Given the name of the protein (also referred to herein as the “full protein”; indicated as “Protein”), other peptide fragments of such measured proteins may be obtained, and such other peptide fragments including those with substantial sequence identity are included within the scope of the invention.

In various embodiments, measuring the expression level of a gene set include measuring the expression level of each gene in the set (including the transcript level, the protein expression level, or both), and a decreased or increased level of the expression level of a gene set include a decreased, or increased respectively, level of each gene in the set. In various embodiments, measuring the expression level of a gene or a plurality of gene in a set comprises or consists of measuring any number of genes in the set (e.g., one, two, three, four, five, . . . up to the total number in the set).

### Treatment/Selection

Various embodiments of the present invention provide for a method of treating a cancer subject, comprising: administering an immune checkpoint inhibitor to a subject in need thereof, wherein the subject has been determined to have a marker gene or a plurality of marker genes of the following relative levels in a set selected from the group consisting of:

i) a plurality of marker genes having at least 95% sequence identity with sequences selected from Table 3, or homologs or variants thereof, each (when selected) having an expression level below its respective reference value;
ii) a plurality of marker genes having at least 95% sequence identity with sequences selected from Table 4, or homologs or variants thereof, wherein TFF1, ANXA10, FCGBP, IL33, TP53I11, TMEM45B, ADAM28, ATF6B, NDUFA4L2, CAPN8, HMCN2, and ALDH3A1 (when selected) each has an expression level below its respective reference value, and NEBL, MLIP, CSMD2, NXPH4, SCNN1B, IGFL1, and DEFB1 (when selected) each has an expression level above its respective reference value;
iii) a plurality of marker genes having at least 95% sequence identity with sequences selected from Table 5, or homologs or variants thereof, each (when selected) having an expression level below its respective reference value;
iv) a plurality of marker genes having at least 95% sequence identity with sequences selected from Table 6, or homologs or variants thereof, wherein GRP, ALAS2, HBA2, MYO15B, HBA1, ALOX15, CXCL6, FRMD5, GABRP, PPARG, CXCL3, CSF2, and CRISP3 (when selected) each has an expression level below its respective reference value, and IL13RA2, ALOX12, TMEM63C, CXCL2, WDR72, GUCY2C, B3GALT2, TRIM66, TPH1, S100A9, ODAPH, and NSUN7 (when selected) each has an expression level above its respective reference value;
v) a polynucleotide which is complementary to any plurality of the marker genes in any one or more of i)-vi);
vi) polypeptides encoded by any plurality of the marker genes in any one or more of i)-vi); and
vii) polypeptides having at least 95% sequence identity to those of vi); whereby the subject is responsive to the immune checkpoint inhibitor.

In some embodiments of the method of treating a cancer subject, the subject has been determined to have a marker gene or a plurality of marker genes of the following relative expression levels:

viii) a plurality of marker genes that have at least 95% sequence identity with sequences selected from Table 7 (Table 7 includes genes from Tables 3 and 5), or homologs or variants thereof, each (when selected) having an expression level below its respective reference value;
ix) a plurality of marker genes that have at least 95% sequence identity with sequences selected from Table 8 (Table 8 includes genes from Tables 4 and 6), or homologs or variants thereof, wherein TFF1, ANXA10, FCGBP, IL33, TP53I11, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, and CRISP3 (when selected) each has an expression level below its respective reference value, and wherein NEBL, MLIP, CSMD2, NXPH4, SCNN1B, IGFL1, DEFB1, IL13RA2, ALOX12, TMEM63C, CXCL2, WDR72, GUCY2C, B3GALT2, TRIM66, TPH1, S100A9, ODAPH, and NSUN7 (when selected) each has an expression level above its respective reference value;
x) a polynucleotide which is complementary to the plurality of the marker genes selected from Table 7 or Table 8, or both;
xi) polypeptides encoded by the plurality of the marker genes of (viii) or (ix); or xii) polypeptides having at least 95% sequence identity to those of xi).

In some embodiments, the marker gene or the plurality of marker genes in set i), ii), iii), iv), viii) or ix) comprises a marker gene that is 100% sequence identity with those set forth in Table 3, 4, 5, 6, 7 or 8, respectively. In some embodiments, the marker gene or the plurality of marker genes in set i), ii), iii), iv), viii) or ix) comprises a marker gene that is 99%, 98%, 97%, 96% or 95% sequence identity with those set forth in Table 3, 4, 5, 6, 7 or 8, respectively. In some embodiments, the marker gene or the plurality of marker genes do not comprise a marker gene that is set forth in Table 1 and 2 but not set forth in any of Tables 3-8. In some embodiments, the marker gene or the plurality of marker genes further comprises a marker gene that is set forth in Table 1 or 2. In some embodiments, the marker gene or the plurality of marker genes in set i), ii), v), vi) or vii) are further accompanied by an upregulated expression of DDR1 or an expression level of DDR1 above a reference value. In some embodiments, the marker gene or the plurality of marker genes in set iii), iv), v), vi) or vii) are further accompanied by a down-regulated expression of DDR2 or an expression level of DDR2 below a reference value.

Various embodiments provide for a method of selecting a cancer patient for administration of an immune checkpoint inhibitor, comprising detecting or measuring in a sample of tumor cells from the patient a level of expression of a plurality of marker genes in one or more sets selected from i)-vii) described above, or a plurality of marker genes in one or more sets selected from viii)-xii), wherein the patient is selected for administration of an immune checkpoint inhibitor when the plurality of genes have a relative expression level compared to respective reference value as described above with the respective set, and the patient is not selected for administration of an immune checkpoint inhibitor when the plurality of genes does not have a relative expression level as described above with the respective set.

Various embodiments of the present invention provide for a method of selecting a cancer patient and treating the subject, comprising: selecting a subject whose tissue expresses a plurality of marker genes selected from one or more sets of i)-vii), or a plurality of marker genes selected from one or more sets of viii)-xii), at a level relative to a reference value as described above with the respective set, and administering an immune checkpoint inhibitor to the subject.

### Detection and Treatment

Various embodiments of the present invention provide for a method for treating cancer in a subject, comprising: measuring the expression level of a plurality of marker genes selected from one or more sets of i)-vii) or one or more sets of viii)-xii) from the cancer tissue of the subject, and administering an immune checkpoint inhibitor to the subject,

wherein if selected, AQP3, NDUFA4L2, PALM, DHRS3, GGT5, GIPR, GALNT18, ANO1, PCDHGB2, LURAP1L, S100A2, GCNT3, CXCL6, MMP10, TFF1, ANXA10, FCGBP, IL33, TP53111, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, and CRISP3 each has an expression level below its respective reference value, and

wherein if selected, NEBL, MLIP, CSMD2, NXPH4, SCNN1B, IGFL1, DEFB1, IL13RA2, ALOX12, TMEM63C, CXCL2, WDR72, GUCY2C, B3GALT2, TRIM66, TPH1, S100A9, ODAPH, and NSUN7 each has an expression level above its respective reference value.

In various embodiments, “the expression pattern” refers to AQP3, NDUFA4L2, PALM, DHRS3, GGT5, GIPR, GALNT18, ANO1, PCDHGB2, LURAP1L, S100A2, GCNT3, CXCL6, MMP10, TFF1, ANXA10, FCGBP, IL33, TP53I11, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, and CRISP3, if selected, each has an expression level below its respective reference value, and NEBL, MLIP, CSMD2, NXPH4, SCNN1B, IGFL1, DEFB1, IL13RA2, ALOX12, TMEM63C, CXCL2, WDR72, GUCY2C, B3GALT2, TRIM66, TPH1, S100A9, ODAPH, and NSUN7, if selected each has an expression level above its respective reference value. In various embodiments, this expression pattern is based on the value of log 2FC (CR vs. PD) for each gene in Tables 3-6, wherein the gene marker's expression in complete responder is lower than that in patient with progression of disease when log 2FC (CR vs. PD)<0, i.e., the fold change of the gene marker in complete responder is lower than that is patient with progression of disease, and accordingly, the gene marker's expression in complete responder is lower than that in patient with progression of disease when log 2FC (CR vs. PD)>0.

Further embodiments of the method provide include discontinuing administration of a therapeutic agent consisting of or consisting essentially of an immune checkpoint inhibitor to the subject if the expression levels of the marker genes relative to respective reference values are not the expression pattern.

In some embodiments, a method for treating bladder cancer in a subject, comprising:

measuring the expression level of a plurality of marker genes selected from one or more sets of:

a) all marker genes set forth in Table 3;

b) all marker genes set forth in Table 4;

c) all marker genes set forth in Table 5;

d) all marker genes set forth in Table 6;

e) polynucleotides which are complementary to any plurality of the marker genes in any of a)-d);

f) polypeptides encoded by any plurality of the marker genes in any of a)-d); or

g) polypeptides have substantial sequence identity with those of f);

and

administering an immune checkpoint inhibitor to the subject,

wherein if selected, AQP3, NDUFA4L2, PALM, DHRS3, GGT5, GIPR, GALNT18, ANO1, PCDHGB2, LURAP1L, S100A2, GCNT3, CXCL6, MMP10, TFF1, ANXA10, FCGBP, IL33, TP53I11, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, and CRISP3 each has an expression level below its respective reference value, and if selected, NEBL, MLIP, CSMD2, NXPH4, SCNN1B, IGFL1, DEFB1, IL13RA2, ALOX12, TMEM63C, CXCL2, WDR72, GUCY2C, B3GALT2, TRIM66, TPH1, S100A9, ODAPH, and NSUN7 each has an expression level above its respective reference value.

Various embodiments of the present invention provide for a method for treating cancer in a subject, comprising:

obtaining or requesting result of an analysis of expression level from a cancer tissue of the subject of:

1) a plurality of marker genes having substantial sequence identity with those set forth in Table 3;

2) a plurality of marker genes having substantial sequence identity with those set forth in Table 4;

3) a plurality of marker genes having substantial sequence identity with those set forth in Table 5;

4) a plurality of marker genes having substantial sequence identity with those set forth in Table 6;

5) polynucleotides which are complementary to any plurality of the marker genes in any of a)-d);

6) polypeptides encoded by any plurality of the marker genes in any of 1)-4); or 7) polypeptides have substantial sequence identity with those of 6);

and

administering an immune checkpoint inhibitor to the subject.

In some embodiments, a method for treating bladder cancer, lung cancer, leukemia, or a combination thereof in a subject, comprising:

obtaining or requesting result of an analysis of expression level from a cancer tissue of the subject of:

a) all marker genes set forth in Table 3;

b) all marker genes set forth in Table 4;

c) all marker genes set forth in Table 5;

d) all marker genes set forth in Table 6;

e) polynucleotides which are complementary to any plurality of the marker genes in any of a)-d);

f) polypeptides encoded by any plurality of the marker genes in any of a)-d); or

g) polypeptides have substantial sequence identity with those of f);

and

administering an immune checkpoint inhibitor to the subject,

wherein if selected, AQP3, NDUFA4L2, PALM, DHRS3, GGT5, GIPR, GALNT18, ANO1, PCDHGB2, LURAP1L, S100A2, GCNT3, CXCL6, MMP10, TFF1, ANXA10, FCGBP, IL33, TP53I11, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, and CRISP3 each has an expression level below its respective reference value, and if selected, NEBL, MLIP, CSMD2, NXPH4, SCNN1B, IGFL1, DEFB1, IL13RA2, ALOX12, TMEM63C, CXCL2, WDR72, GUCY2C, B3GALT2, TRIM66, TPH1, S100A9, ODAPH, and NSUN7 each has an expression level above its respective reference value.

Further embodiments provide for a method of treating cancer in a subject having been treated with an immune checkpoint inhibitor, comprising: measuring the expression level of marker genes selected from one or more sets of any of i)-xii) from the cancer tissue of the subject, and continuing to administer an immune checkpoint inhibitor to the subject if the expression level of marker genes follow the expression pattern, or discontinuing the administration of the immune checkpoint inhibitor to the subject if the expression level of the marker genes are not the expression pattern.

In various embodiments, the cancer comprises bladder cancer, and the cancer tissue comprises bladder cancer tissue. In some embodiments, the cancer comprises lung cancer, and the cancer tissue comprises lung cancer tissue. In other embodiments, the cancer comprises hematologic cancer such as leukemia, and the cancer tissue comprises bone marrow and/or lymphatic system specimen.

In various embodiments, the expression level of markers is obtained from a tissue of the subject, wherein the tissue is the cancerous tissue, such as cancerous bladder tissue. In other embodiments, the tissue is a bladder tissue, wherein the bladder has cancer. In further embodiments, normal tissue adjacent to the tumor is measured as a control. In some embodiments, the bladder tissue where the expression level of markers is obtained is a cancerous bladder tissue.

In some embodiments, the level of the markers is compared to a standard level or a reference level. Typically, the standard biomarker level or reference range is obtained by measuring the same marker or markers in a set of normal controls. In various embodiments, the reference value is the expression level in a control subject, e.g., non-cancer subject or one treated to be free of cancer, and of the same mammalian species as the subject for treatment in the method. In various embodiments, the decreased, lowered, or higher or greater level of expression in the subject in the methods is compared to the level of expression from a non-cancerous tissue of the same type of organ from a control subject, wherein the control subject does not have the cancer. In various embodiments, the decreased or lowered level of expression is compared to the average level of expression from a non-cancerous tissue of the same type of organ from a group of subjects that do not have the cancer. In various embodiments, the decreased or lowered level of expression is compared to the level of expression from tissue of the same organ from the subject before signs or symptoms of cancer show up. In other embodiments, the reference value is the median expression level of the respective marker gene in a pool or database of cancer tissues from subjects with the same cancer type. Measurement of the standard biomarker level or reference range need not be made contemporaneously; it may be a historical measurement. Preferably the normal control is matched to the patient with respect to some attribute(s) (e.g., age). Depending upon the difference between the measured and standard level or reference range, the patient can be diagnosed as predicted to respond to the immune checkpoint inhibitor therapy or as not predicted to respond to the immune checkpoint inhibitor therapy.

Examples of immune checkpoint inhibitors, or immune checkpoint blockade (ICB) therapeutics, include but are not limited to, an anti-PD-L1 antibody, an antibody against PD-1, an antibody against PD-L2, an antibody against CTLA-4, an antibody against KIR, an antibody against IDO1, an antibody against ID02, an antibody against TIM-3, an antibody against LAG-3, an antibody against OX40R, and an antibody against PS.

Other examples of immune checkpoint inhibitors include inhibitors of leukocyte surface antigen CD47 (antigenic surface determinant protein OA3 or integrin associated protein or protein MER6 or CD47), and such examples are magrolimab (by Forty Seven), IBI-188 (by Innovent Biologics), ALX-148 (by ALX Oncology), AO-176 (by Arch Oncology), and CC-90002 (by Bristol-Myers Squibb).

Another class of exemplary immune checkpoint inhibitors or immune checkpoint blockade therapeutics include antagonists or inhibitors of T cell immunoreceptor with Ig and ITIM domains (V set and immunoglobulin domain containing protein 9 or V set and transmembrane domain containing protein 3 or TIGIT), and such examples are tiragolumab (by Genentech), AB-154 (by Arcus Biosciences), BMS-986207 (by Bristol-Myers Squibb), vibostolimab (by Merck), and BGBA-1217 (by BeiGene).

Yet another class of exemplary immune checkpoint inhibitors or immune checkpoint blockade therapeutics include antagonists of adenosine receptor A2a (ADORA2A) or A2b (ADORA2B), and examples include AB-928 (by Arcus Biosciences), ciforadenant (by Corvus Pharmaceuticals), HTL-1071 (by AstraZeneca), PBF-509 (by Novartis), and EOS-100850 (by iTeos Therapeutics).

In one embodiment, the immune checkpoint inhibitor is humanized monoclonal anti-programmed death ligand 1 (PD-L1) antibody, atezolizumab. In another embodiment, the immune checkpoint inhibitor is an anti-PD-L1 antibody such as avelumab, durvalumab, KN035, CK-301, AUNP12, CA-170, MPDL3280A(RG7446), MEDI4736 and BMS-936559.

In another embodiment, the immune checkpoint inhibitor is an anti-PD-1 antibody such as pembrolizumab (formerly lambrolizumab or MK-3475), nivolumab (BMS-936558), cemiplimab, spartalizumab, camrelizumab, sintilimab, tislelizumab, toripalimab, Pidilizumab (CT-011), AMP-224, or AMP-514.

Further examples of immune checkpoint inhibitor, or immune checkpoint blockade (ICB) therapeutics, include but are not limited to, B7-DC-Fc fusion proteins such as AMP-224, anti-CTLA-4 antibodies such as tremelimumab (CP-675,206) and ipilimumab (MDX-010), antibodies against the B7/CD28 receptor superfamily, anti-Indoleamine (2,3)-dioxygenase (IDO) antibodies, anti-IDO1 antibodies, anti-ID02 antibodies, tryptophan, tryptophan mimetic, 1-methyl tryptophan (1-MT)), Indoximod (D-1-methyl tryptophan (D-1-MT)), L-1-methyl tryptophan (L-1-MT), TX-2274, hydroxyamidine inhibitors such as INCB024360, anti-TIM-3 antibodies, anti-LAG-3 antibodies such as BMS-986016, recombinant soluble LAG-3Ig fusion proteins that agonize MHC class II-driven dendritic cell activation such as IMP321, anti-KIR2DL1/2/3 or anti-KIR) antibodies such lirilumab (IPH2102), urelumab (BMS-663513), anti-phosphatidylserine (anti-PS) antibodies such as Bavituximab, anti-idiotype murine monoclonal antibodies against the human monoclonal antibody for N-glycolil-GM3 ganglioside such as Racotumomab (formerly known as 1E10), anti-OX40R antibodies such as IgG CD134 mAb, anti-B7-H3 antibodies such as MGA271, and small interfering (si) RNA-based cancer vaccines designed to treat cancer by silencing immune checkpoint genes.

In various embodiments, the immune checkpoint inhibitor is formulated into a pharmaceutical composition. Pharmaceutical compositions according to the invention may be formulated for delivery via any route of administration.

“Route of administration” may refer to any administration pathway known in the art, including but not limited to parenteral, aerosol, nasal, oral, transmucosal, or transdermal. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. “Transdermal” administration may be accomplished using a topical cream or ointment or by means of a transdermal patch. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions based on immune checkpoint inhibitors may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication.

The immune checkpoint inhibitors of the methods can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The immune checkpoint inhibitors of the methods can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for Tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins P A, USA) (2000).

In further embodiments, the therapeutic methods of the present invention may be combined with other anti-cancer therapies. Examples of anti-cancer therapies include traditional cancer treatments such as surgery, chemoradiation, anticancer drugs (e.g., cisplatin, carboplatin, oxaliplatin, nedaplatin, triplatin tetranitrate, phenanthriplatin, picoplatin, satraplatin), taxane, anti-VEGF therapy, as well as other new treatments. Such other anti-cancer therapies will be expected to act in an additive or synergistic manner with the immune checkpoint blockade therapy.

Yet in further embodiments of the methods, the immune checkpoint blockade therapies may be combined with, in combination with, or administered to a patient having received or in need of receiving, an inhibitor of DDR2, or an inhibitor of DDR. Examples of inhibitor of DDR2 include receptor tyrosine kinase inhibitors such as dasatinib, imatinib, nilotinib, and ponatinib.

When two or more therapies are administered in combination, they may be administered sequentially, concurrently, or even in a pre-mix composition, or even separated by a period of time.

### Detection, Analysis, and Prognosis

Various embodiments provide for a method of detecting bladder cancer in a subject, comprising: obtaining a bladder tissue sample from a subject in need thereof, and measuring the expression levels of DDR1 and DDR2 from the sample, wherein (1) an expression level of DDR1 higher than a standardized 50 percentile value of DDR1 expression from a reference group of subjects and an expression level of DDR2 lower than a standardized 50 percentile value of DDR2 expression from the reference group indicates the subject has bladder cancer, or (2) an expression level of DDR1 lower than a standardized 50 percentile value of DDR1 expression from a reference group of subjects and an expression level of DDR2 higher than a standardized 50 percentile value of DDR2 expression from the reference group indicates the subject has bladder cancer.

In some embodiments, there are two distinct types of bladder cancer including (1) DDR1-^{high}; DDR2-^{low} and (2) DDR1^{low}; DDR2^{high}. Standardized 50 percentile values of DDR1 and DDR2 were zero as a cutoff level both of DDR1 and DDR2 expression across the samples from the TCGA tumors. In some embodiments, the reference group is a plurality of subjects with TCGA tumors. The subject can be defined as one of the two types with this cutoff value of DDR1 and DDR2. In some embodiments, the indicated bladder cancer is a TNM stage 2 bladder cancer or of a higher than 2 stage. In further embodiments, the bladder cancer is indicated to be LumP subtype when it has a high fraction (>50%) in the bladder tissue sample of DDR1^{high} and a high fraction (>50%) of DDR2^{low}; the bladder cancer is indicated to be Stromal-rich subtype when it has a high fraction (>50%) in the bladder tissue sample of DDR2^{high} and a high fraction (>50%) of DDR1^{low}. Bladder cancer can be categorized into at least six subtypes on molecular level, called Luminal papillary (LumP), Basal/squamous (Ba/Sq), Luminal unstable (LumU), Stromal-rich, Luminal non-specified (LumNS), and Neuroendocrine-like (NE-like), as described by Jalanko T. et al., in Current Urology Reports, 21, 9, (2020), which is incorporated by reference herein. In further embodiments, DDR2^{high} tumors are positively correlated with immune cells including B cells, T cells, dendritic cells, macrophages, monocytes and NK cells in the bladder tumor microenvironment (bTME); and DDR1^{high} tumors are negatively correlated with B cells, T cells, dendritic cells, macrophages, monocytes or NK cells in the bladder tumor microenvironment (no significant detection of these immune cells), but for memory and naïve CD4+ T cells, mast cells and neutrophils. The presence or absence of the B cells, T cells, dendritic cells, macrophages, monocytes and/or NK cells in the bladder tumor microenvironment can also be used to determine DDR2^{high} or DDR1^{high} tumors, respectively. In various embodiments, a subject with bladder cancer determined to be DDR2^{low} has a better survival prognosis than a subject with bladder cancer determined to be DDR2^{high}.

Various embodiments provide for a method of detecting the presence or absence of bladder cancer in a subject, comprising: obtaining a bladder tissue sample from a subject in need thereof, and measuring the expression levels of DDR1 and DDR2 from a bladder tissue of a subject desiring to determine whether bladder cancer is present, or whether bladder cancer will develop.

Various embodiments provide for a method of detecting a level of DDR1, DDR2, or both in a subject, comprising: assaying a biological sample obtained from the subject, wherein the subject desires a determination regarding cancer or exhibits a symptom of the cancer, and detecting the level of DDR1, DDR2, or both.

Various embodiments provide for a method of detecting expression levels of a plurality of marker genes in a subject, comprising: assaying a biological sample obtained from the subject, wherein the subject desires a prognosis of cancer following or before receiving an immune checkpoint blockade therapy, and detecting the expression levels of a plurality of marker genes selected from:

1) a plurality of marker genes having substantial sequence identity with those set forth in Table 3;

2) a plurality of marker genes having substantial sequence identity with those set forth in Table 4;

3) a plurality of marker genes having substantial sequence identity with those set forth in Table 5;

4) a plurality of marker genes having substantial sequence identity with those set forth in Table 6;

5) polynucleotides which are complementary to any plurality of the marker genes in any of a)-d);

6) polypeptides encoded by any plurality of the marker genes in any of 1)-4); or

7) polypeptides have substantial sequence identity with those of 6).

set i), ii) or viii) in the bladder tissue of a subject desiring to determine whether an immune checkpoint inhibitor is effective in treating bladder cancer of the subject.

In some embodiments, a method of detecting expression levels of a plurality of marker genes in a subject, comprising: assaying a bladder tissue sample obtained from the subject, wherein the subject desires to determine whether an immune checkpoint inhibitor is effective in treating bladder cancer of the subject, and detecting the expression levels of a plurality of marker genes selected from:

a) all marker genes set forth in Table 3;

b) all marker genes set forth in Table 4;

c) all marker genes set forth in Table 5;

d) all marker genes set forth in Table 6;

e) polynucleotides which are complementary to any plurality of the marker genes in any of a)-d);

f) polypeptides encoded by any plurality of the marker genes in any of a)-d); or

g) polypeptides have substantial sequence identity with those of f).

In some embodiments, a method of detecting expression levels of a plurality of marker genes in a subject, comprising: assaying a lung tissue sample obtained from the subject, wherein the subject desires to determine whether an immune checkpoint inhibitor is effective in treating lung cancer of the subject, and detecting the expression levels of a plurality of marker genes selected from:

a) all marker genes set forth in Table 3;

b) all marker genes set forth in Table 4;

c) all marker genes set forth in Table 5;

d) all marker genes set forth in Table 6;

e) polynucleotides which are complementary to any plurality of the marker genes in any of a)-d);

f) polypeptides encoded by any plurality of the marker genes in any of a)-d); or

g) polypeptides have substantial sequence identity with those of f).

Various embodiments provide for a method of detecting the presence or absence of a marker gene or a plurality of marker genes in set iii), iv) or xi) in bladder tissue, comprising: obtaining a bladder tissue sample from a subject in need thereof; and measuring the expression levels of the marker gene or the plurality of marker genes selected from iii), iv) or xi) in the bladder tissue of a subject desiring to determine whether an immune checkpoint inhibitor is effective in treating bladder cancer of the subject.

Further embodiments provide for a method of detecting the presence or absence by measuring the expression level of a marker gene or a plurality of marker genes in any set of i)-xii) in bladder tissue, comprising: obtaining a bladder tissue sample from a subject in need thereof; and measuring the expression levels of a marker gene or a plurality of marker genes in any set of i)-xii) in the bladder tissue of a subject desiring to determine whether an immune checkpoint inhibitor is effective in treating bladder cancer of the subject.

In some embodiments, an immune checkpoint inhibitor is effective in treating bladder cancer when there is at least 10%, 20%, 30%, 40%, 50% or more of reduction in symptoms or pathology of bladder cancer after an effective amount of the immune checkpoint inhibitor is administered to the subject in need thereof. In some embodiments, a subject's responsiveness to an agent in treating a disease or condition is the amount of reduction in symptoms or pathology of the disease or condition with the administration of the agent; and improving or increasing a subject's responsiveness to an agent in treating a disease or condition indicates the subject's symptoms or pathology of the disease or condition is reduced with the administration of the agent, compared to the symptoms or pathology prior to the administration of the agent.

In various embodiments, the subject has a cancer. In some embodiments, the subject has a bladder cancer. In various embodiments, the subject is suspected of having bladder cancer. In further embodiments, the subject has a bladder cancer and receives administration of an inhibitor of DDR2.

Further embodiments provide methods of determining risk associated with cancer in a cancer patient, comprising measuring expression levels of a set of signature genes in cancer cells of said cancer patient, thereby obtaining a risk score (RS) based on the expression levels of said set of signature genes, and determining risk of cancer for said cancer patient by comparing the risk score to a predefined risk score cut off threshold for said set of signature genes.

In some embodiments, methods of treating a cancer patient determined of risk associated with cancer or of responsiveness to an ongoing therapy, comprise measuring expression levels of a set of signature genes in cancer cells of the cancer patient, thereby obtaining a risk score (RS) based on the expression levels of said set of signature genes, wherein the cancer patient is determined to have an increased risk of poor survival (or at risk of deterioration) or determined to be non-responsive to the ongoing therapy if his/her RS is higher than a predefined RS cut off threshold, or wherein the cancer patient is determined to have a decreased risk of poor survival or determined to be responsive to the ongoing therapy if his/her RS is lower than the predefined RS cut off threshold; and administering an immune checkpoint inhibitor to the patient determined to have a decreased risk of poor survival or determined to be responsive to the ongoing therapy, or preventing the patient determined to have an increased risk of poor survival or determined to be non-responsive from the ongoing therapy from receiving an immune checkpoint inhibitor or from receiving the ongoing therapy, respectively. In some embodiments, poor survival may refer to worse survival outcome compared to a survival parameter of a control or of pool of patients with same type of cancer.

In some embodiments, the RS score is a Z-score which represents the difference (in standard deviations) between the error-weighted mean of the expression values of the genes in a signature (or in a pathway) and the error-weighted mean of all genes in a sample after normalization. It is a score that reflects both the magnitude and relative direction of a gene set's expression. A Z-score metric to measure the relative expression level of signature S in tissue t is:

$${Z}_{tS} = \frac{〈 {X}_{tS} 〉 - 〈 {X}_{t} 〉}{{σ}_{t}} \sqrt{\vert S \vert} ,$$

where |S| is the number of genes in S, at <X_{tS}> is the mean of X_{tg} over the genes in S, and <Xₜ> is the mean of X_{tg} over all the genes assayed (e.g., on a microarray), X_{tg} is the expression value (log 10 fold change, relative to background) for a given gene g, and σₜ is the standard deviation of X_{tg} over all the genes assayed. In further embodiments, the RS score is a Wilcoxon Z statistic, which is calculated according to a similar formula, but using the ranks of the X_{tg} among all genes in tissue t, rather than the actual fold changes. Further description is seen in Levine, D. M., et al., Genome Biol. 2006; 7(10):R93, which is incorporated by reference herein.

In some embodiments, the RS score is based on multivariable Cox proportional hazard regression analysis (Cox model), which investigate the clinical association between risk score panel genes and the overall survival. The RS score of each patient is calculated as: risk score=Σᵢ₌₁ⁿ(βᵢ×xᵢ), where n is the number of genes in the risk score panel, βᵢ is the coefficient of ith gene modeled by Cox proportional hazard regression analysis, and xᵢ is the median centered gene expression of ith gene.

### Stratification/Assessment

Various embodiments provide for a method of determining if a cancer patient is predicted to respond to the administration of an immune checkpoint blockade therapy, comprising: detecting or measuring in a sample of tumor cells from the patient a level of expression of a plurality of marker genes selected from:

1) a plurality of marker genes having substantial sequence identity with those set forth in Table 3;

2) a plurality of marker genes having substantial sequence identity with those set forth in Table 4;

3) a plurality of marker genes having substantial sequence identity with those set forth in Table 5;

4) a plurality of marker genes having substantial sequence identity with those set forth in Table 6;

5) polynucleotides which are complementary to any plurality of the marker genes in any of 1)-4);

6) polypeptides encoded by any plurality of the marker genes in any of 1)-4); or

7) polypeptides have substantial sequence identity with those of 6),

wherein the patient is indicated to respond to the administration of an immune checkpoint blockade therapy when AQP3, NDUFA4L2, PALM, DHRS3, GGT5, GIPR, GALNT18, ANO1, PCDHGB2, LURAP1L, S100A2, GCNT3, CXCL6, MMP10, TFF1, ANXA10, FCGBP, IL33, TP53I11, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, and CRISP3, if selected, each has an expression level below its respective reference value, and NEBL, MLIP, CSMD2, NXPH4, SCNN1B, IGFL1, DEFB1, IL13RA2, ALOX12, TMEM63C, CXCL2, WDR72, GUCY2C, B3GALT2, TRIM66, TPH1, S100A9, ODAPH, and NSUN7, if selected, each has an expression level above its respective reference value.

In some embodiments, the patient indicated to respond to the immune checkpoint blockade therapy has a bladder cancer of Luminal papillary (LumP) subtype. In some embodiments, the patient indicated to respond to the immune checkpoint blockade therapy has a bladder cancer of Basal/squamous (Ba/Sq) subtype. In some embodiments, the patient indicated to respond to the immune checkpoint blockade therapy has a bladder cancer of Luminal unstable (LumU) subtype. In some embodiments, the patient indicated to respond to the immune checkpoint blockade therapy has a bladder cancer of Stromal-rich subtype. In some embodiments, the patient indicated to respond to the immune checkpoint blockade therapy has a bladder cancer of Luminal non-specified (LumNS) subtype. In some embodiments, the patient indicated to respond to the immune checkpoint blockade therapy has a bladder cancer of Neuroendocrine-like (NE-like) subtype.

Various embodiments provide for a method of monitoring the progression of cancer, or assessing the efficacy or effectiveness of an immune checkpoint blockade therapy being administered to a cancer subject, comprising: comparing the expression level of a plurality of marker genes measured in a first sample obtained from the subject at a time t0, with the expression level of the plurality of marker genes measured in a second sample obtained from the subject at a time t1, said t1 is after said t0, wherein the plurality of marker genes is selected from:

1) a plurality of marker genes having substantial sequence identity with those set forth in Table 3;

2) a plurality of marker genes having substantial sequence identity with those set forth in Table 4;

3) a plurality of marker genes having substantial sequence identity with those set forth in Table 5;

4) a plurality of marker genes having substantial sequence identity with those set forth in Table 6;

5) polynucleotides which are complementary to any plurality of the marker genes in any of 1)-4);

6) polypeptides encoded by any plurality of the marker genes in any of 1)-4); or

7) polypeptides have substantial sequence identity with those of 6),

and wherein the immune checkpoint blockade therapy is indicated to be effective for treating the cancer in the subject when AQP3, NDUFA4L2, PALM, DHRS3, GGT5, GIPR, GALNT18, ANO1, PCDHGB2, LURAP1L, S100A2, GCNT3, CXCL6, MMP10, TFF1, ANXA10, FCGBP, IL33, TP53111, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, and CRISP3, if selected, each has a lower expression level at t1 compared to respective expression level at t0, and NEBL, MLIP, CSMD2, NXPH4, SCNN1B, IGFL1, DEFB1, IL13RA2, ALOX12, TMEM63C, CXCL2, WDR72, GUCY2C, B3GALT2, TRIM66, TPH1, S100A9, ODAPH, and NSUN7, if selected, each has a higher expression level at t1 compared to respective expression level at t0, or wherein the immune checkpoint inhibitor is indicated to be effective in treating the cancer in the subject when a risk score of the patient based on the expression level of the selected plurality of marker genes at the time t1 is lower than that at the time t0.

In some aspects, t0 is before the subject's receiving of an immune checkpoint blockade therapy, and t1 is after the subject has received the immune checkpoint blockade therapy. In other aspects, both t0 and t1 are after the subject receives the immune checkpoint blockade.

In a further embodiment, a method of monitoring the progression of cancer, or assessing the efficacy or effectiveness of an immune checkpoint blockade therapy being administered to a bladder cancer subject, comprising: comparing the expression level of a plurality of marker genes measured in a first bladder tumor sample obtained from the subject at a time t0, with the expression level of the plurality of marker genes measured in a second bladder tumor sample obtained from the subject at a time t1, said t1 is after said t0, wherein the plurality of marker genes is selected from:

a) a plurality of marker genes set forth in Table 3;

b) a plurality of marker genes set forth in Table 4;

c) a plurality of marker genes set forth in Table 5;

d) a plurality of marker genes set forth in Table 6;

e) polynucleotides which are complementary to any plurality of the marker genes in any of a)-d);

f) polypeptides encoded by any plurality of the marker genes in any of a)-d); or

g) polypeptides have substantial sequence identity with those of f).

and wherein the immune checkpoint blockade therapy is indicated to be effective for treating the bladder cancer in the subject when AQP3, NDUFA4L2, PALM, DHRS3, GGT5, GIPR, GALNT18, ANO1, PCDHGB2, LURAP1L, S100A2, GCNT3, CXCL6, MMP10, TFF1, ANXA10, FCGBP, IL33, TP53I11, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, and CRISP3, if selected, each has a lower expression level at t1 compared to respective expression level at t0, and NEBL, MLIP, CSMD2, NXPH4, SCNN1B, IGFL1, DEFB1, IL13RA2, ALOX12, TMEM63C, CXCL2, WDR72, GUCY2C, B3GALT2, TRIM66, TPH1, S100A9, ODAPH, and NSUN7, if selected, each has a higher expression level at t1 compared to respective expression level at t0.

In some embodiments, the time t0 is before the treatment has been administered to the subject, and the time t1 is after the treatment has been administered to the subject. In some embodiments, the comparing is repeated over a range of times.

In various embodiments, an immune checkpoint blockade therapy includes an immune checkpoint inhibitor.

### Screening Methods for Therapeutics

Various embodiments provide for a method of identifying an agent effective for treating bladder cancer, and/or improving a subject's responsiveness to an immune checkpoint inhibitor in the treatment of a bladder cancer or another tumor in the subject, comprising: contacting a molecule of interest with cells or tissues derived from bladder or tumor tissue, measuring the expression level of a plurality of marker genes in the cells or tissues in the presence of the molecule of interest, and measuring the expression level of the plurality of marker genes in the cells or tissues before the contact with or in the absence of the molecule of interest, wherein the plurality of marker genes is selected rom

1) a plurality of marker genes having substantial sequence identity with those set forth in Table 3;

2) a plurality of marker genes having substantial sequence identity with those set forth in Table 4;

3) a plurality of marker genes having substantial sequence identity with those set forth in Table 5;

4) a plurality of marker genes having substantial sequence identity with those set forth in Table 6;

5) polynucleotides which are complementary to any plurality of the marker genes in any of 1)-4);

6) polypeptides encoded by any plurality of the marker genes in any of 1)-4); or

7) polypeptides have substantial sequence identity with those of 6),

and wherein a decrease in the expression level of AQP3, NDUFA4L2, PALM, DHRS3, GGT5, GIPR, GALNT18, ANO1, PCDHGB2, LURAP1L, S100A2, GCNT3, CXCL6, MMP10, TFF1, ANXA10, FCGBP, IL33, TP53111, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, and CRISP3, if selected, in the presence of the molecule of interest compared to that before the contact with or in the absence of the molecule of interest indicates that the molecule is an agent effective for treating the bladder cancer or other tumor and/or improving a subject's responsiveness to an immune checkpoint inhibitor in the treatment of the bladder cancer or other tumor.

### Assay System

Various embodiments provide for an assay system for predicting patient response or outcome to immune checkpoint blockade therapy for cancer comprising nucleic acid probes that comprise complementary nucleic acid sequences to at least 10 to 50 nucleic acid sequences of a plurality of marker genes selected rom

1) a plurality of marker genes having substantial sequence identity with those set forth in Table 3;

2) a plurality of marker genes having substantial sequence identity with those set forth in Table 4;

3) a plurality of marker genes having substantial sequence identity with those set forth in Table 5;

4) a plurality of marker genes having substantial sequence identity with those set forth in Table 6; and/or

5) polynucleotides which are complementary to any plurality of the marker genes in any of 1)-4);

so as to measure the expression level (e.g., mRNA level, or protein level) of the genes. In one embodiment, the expression level is the RNA level. In one embodiment, the expression level is the protein level. In one embodiment, the expression level includes RNA and protein levels. In other embodiments, an assay system for predicting patient response or outcome to immune checkpoint blockade therapy for cancer comprises binding ligands that specifically detect polypeptides encoded by the plurality of marker genes.

Further embodiments provide the assay system further comprises an assay surface such as a chip, array, or fluidity card.

Exemplary reagents or molecules which specifically bind the marker (gene or polypeptide encoded by the gene), e.g., binding ligand, include but are not limited to antibodies, aptamers and antibody derivatives or fragments.

### Biological Samples

In various embodiments, the biological sample is bladder tissue. In various embodiments, the bladder tissue is cancerous bladder tissue. In further embodiments, normal tissue adjacent to the tumor is measured as a control.

Additional examples of biological samples include but are not limited to body fluids, cancer cells in body fluids isolated by any technique, free RNA and protein in body fluids such as but not limited to whole blood, plasma, stool, intestinal fluids or aspirate, intestinal mucosal biopsies, serum, cerebral spinal fluid (CSF), urine, saliva, pulmonary secretions, breast aspirate, prostate fluid, seminal fluid, cervical scraping, amniotic fluid, mucous, and moisture in breath. In some embodiments of the method, the biological sample may be whole blood, blood plasma, blood serum, stool, intestinal fluid or aspirate or stomach fluid or aspirate. In some embodiments, the biological sample is blood, plasma, and/or urine.

Detection/Measurement techniques

In various embodiments, measurements or detection of gene expression can be performed by extracting RNA/DNA from tissue specimens, obtaining polypeptides (including proteins) from the biological sample, measuring UV absorption with a spectrophotometer, gel electrophoresis coupled with biochemical or luminescent quantification, and/or whole/partial genome amplification. In various embodiments, the expression protein levels are measured using one or more of these techniques.

In various embodiments, measurements or detection of receptor activation can be performed by activation/phosphorylation-specific antibody labeling coupled with gel electrophoresis for quantification.

TABLE 1
Full list of DDR1-overexpression related genes:

| Entrez | | |
|---|---|---|
| Gene ID | Symbol | Description |
| 7031 | TFF1 | trefoil factor 1 |
| 100507487 | LINC02615 | long intergenic non-protein coding RNA 2615 |
| 11199 | ANXA10 | annexin A10 |
| 8857 | FCGBP | Fc fragment of IgG binding protein |
| 90865 | IL33 | interleukin 33 |
| 441058 | LINC01667 | long intergenic non-protein coding RNA 1667 |
| 10529 | NEBL | nebulette |
| 9537 | TP53I11 | tumor protein p53 inducible protein 11 |
| 120224 | TMEM45B | transmembrane protein 45B |
| 90523 | MLIP | muscular LMNA interacting protein |
| 1201 | CLN3 | CLN3 lysosomal/endosomal transmembrane protein, battenin |
| 10863 | ADAM28 | ADAM metallopeptidase domain 28 |
| 114784 | CSMD2 | CUB and Sushi multiple domains 2 |
| 1388 | ATF6B | activating transcription factor 6 beta |
| 56901 | NDUFA4L2 | NDUFA4 mitochondrial complex associated like 2 |
| 11247 | NXPH4 | neurexophilin 4 |
| 388743 | CAPN8 | calpain 8 |
| 256158 | HMCN2 | hemicentin 2 |
| 6338 | SCNN1B | sodium channel epithelial 1 subunit beta |
| 50614 | GALNT9 | polypeptide N-acetylgalactosaminyltransferase 9 |
| 218 | ALDH3A1 | aldehyde dehydrogenase 3 family member A1 |
| 374918 | IGFL1 | IGF like family member 1 |
| 1672 | DEFB1 | defensin beta 1 |
| 360 | AQP3 | aquaporin 3 (Gill blood group) |
| 1116 | CHI3L1 | chitinase 3 like 1 |
| 27132 | CPNE7 | copine 7 |
| 5081 | PAX7 | paired box 7 |
| 1113 | CHGA | chromogranin A |
| 5625 | PRODH | proline dehydrogenase 1 |
| 10516 | FBLN5 | fibulin 5 |
| 83872 | HMCN1 | hemicentin 1 |
| 7033 | TFF3 | trefoil factor 3 |
| 4046 | LSP1 | lymphocyte specific protein 1 |
| 2261 | FGFR3 | fibroblast growth factor receptor 3 |
| 25803 | SPDEF | SAM pointed domain containing ETS transcription factor |
| 136288 | C7orf57 | chromosome 7 open reading frame 57 |
| 1396 | CRIP1 | cysteine rich protein 1 |
| 4549 | RNR1 | s-rRNA |
| 10551 | AGR2 | anterior gradient 2, protein disulphide isomerase family member |
| 90993 | CREB3L1 | cAMP responsive element binding protein 3 like 1 |
| 94234 | FOXQ1 | forkhead box Q1 |
| 3485 | IGFBP2 | insulin like growth factor binding protein 2 |
| 10595 | ERN2 | endoplasmic reticulum to nucleus signaling 2 |
| 102724788 | LOC102724788 | proline dehydrogenase 1, mitochondrial |
| 771 | CA12 | carbonic anhydrase 12 |
| 2304 | FOXE1 | forkhead box E1 |
| 221261 | NaN | NaN |
| 81693 | AMN | amnion associated transmembrane protein |
| 7356 | SCGB1A1 | secretoglobin family 1A member 1 |
| 1917 | EEF1A2 | eukaryotic translation elongation factor 1 alpha 2 |
| 5797 | PTPRM | protein tyrosine phosphatase receptor type M |
| 3560 | IL2RB | interleukin 2 receptor subunit beta |
| 57537 | SORCS2 | sortilin related VPS10 domain containing receptor 2 |
| 51297 | BPIFA1 | BPI fold containing family A member 1 |
| 10811 | NOXA1 | NADPH oxidase activator 1 |
| 283848 | CES4A | carboxylesterase 4A |
| 84969 | TOX2 | TOX high mobility group box family member 2 |
| 1E+08 | MRPL23-AS1 | MRPL23 antisense RNA 1 |
| 122622 | ADSS1 | adenylosuccinate synthase 1 |
| 57016 | AKR1B10 | aldo-keto reductase family 1 member B10 |
| 1953 | MEGF6 | multiple EGF like domains 6 |
| 4550 | RNR2 | l-rRNA |
| 64714 | PDIA2 | protein disulfide isomerase family A member 2 |
| 10562 | OLFM4 | olfactomedin 4 |
| 83481 | EPPK1 | epiplakin 1 |
| 139065 | SLITRK4 | SLIT and NTRK like family member 4 |
| 284339 | TMEM145 | transmembrane protein 145 |
| 54848 | ARHGEF38 | Rho guanine nucleotide exchange factor 38 |
| 8170 | SLC14A2 | solute carrier family 14 member 2 |
| 5064 | PALM | paralemmin |
| 130576 | LYPD6B | LY6/PLAUR domain containing 6B |
| 7140 | TNNT3 | troponin T3, fast skeletal type |
| 151473 | SLC16A14 | solute carrier family 16 member 14 |
| 643596 | RNF224 | ring finger protein 224 |
| 93429 | LOC93429 | uncharacterized LOC93429 |
| 7051 | TGM1 | transglutaminase 1 |
| 1555 | CYP2B6 | cytochrome P450 family 2 subfamily B member 6 |
| 4883 | NPR3 | natriuretic peptide receptor 3 |
| 80726 | IQCN | IQ motif containing N |
| 101927501 | PINCR | p53-induced noncoding RNA |
| 9145 | SYNGR1 | synaptogyrin 1 |
| 5909 | RAP1GAP | RAP1 GTPase activating protein |
| 4586 | MUC5AC | mucin 5AC, oligomeric mucus/gel-forming |
| 105379194 | LOC105379194 | uncharacterized LOC105379194 |
| 23584 | VSIG2 | V-set and immunoglobulin domain containing 2 |
| 163688 | CALML6 | calmodulin like 6 |
| 84152 | PPP1R1B | protein phosphatase 1 regulatory inhibitor subunit 1B |
| 6340 | SCNN1G | sodium channel epithelial 1 subunit gamma |
| 810 | CALML3 | calmodulin like 3 |
| 780 | DDR1 | discoidin domain receptor tyrosine kinase 1 |
| 283120 | H19 | H19 imprinted maternally expressed transcript |
| 79812 | MMRN2 | multimerin 2 |
| 389197 | C4orf50 | chromosome 4 open reading frame 50 |
| 6317 | SERPINB3 | serpin family B member 3 |
| 135 | ADORA2A | adenosine A2a receptor |
| 84871 | AGBL4 | ATP/GTP binding protein like 4 |
| 56704 | JPH1 | junctophilin 1 |
| 1152 | CKB | creatine kinase B |
| 2078 | ERG | ETS transcription factor ERG |
| 146336 | SSTR5-AS1 | SSTR5 antisense RNA 1 |
| 28 | ABO | alpha 1-3-N-acetylgalactosaminyltransferase & alpha 1-3- |
| | | galactosyltransferase |
| 343578 | ARHGAP40 | Rho GTPase activating protein 40 |
| 55068 | ENOX1 | ecto-NOX disulfide-thiol exchanger 1 |
| 401546 | C9orf152 | chromosome 9 open reading frame 152 |
| 341 | APOC1 | apolipoprotein C1 |
| 25825 | BACE2 | beta-secretase 2 |
| 401093 | MBNL1-AS1 | MBNL1 antisense RNA 1 |
| 1556 | CYP2B7P | cytochrome P450 family 2 subfamily B member 7, pseudogene |
| 100500872 | MIR3911 | microRNA 3911 |
| 4143 | MAT1A | methionine adenosyltransferase 1A |
| 2657 | GDF1 | growth differentiation factor 1 |
| 140825 | NEURL2 | neutralized E3 ubiquitin protein ligase 2 |
| 57161 | PELI2 | pellino E3 ubiquitin protein ligase family member 2 |
| 8581 | LY6D | lymphocyte antigen 6 family member D |
| 644809 | C15orf56 | chromosome 15 open reading frame 56 |
| 340113 | LINC02120 | long intergenic non-protein coding RNA 2120 |
| 2036 | EPB41L1 | erythrocyte membrane protein band 4.1 like 1 |
| 4210 | MEFV | MEFV innate immunity regulator, pyrin |
| 342574 | KRT27 | keratin 27 |
| 3860 | KRT13 | keratin 13 |
| 115650 | TNFRSF13C | TNF receptor superfamily member 13C |
| 161247 | FITM1 | fat storage inducing transmembrane protein 1 |
| 4582 | MUC1 | mucin 1, cell surface associated |
| 6899 | TBX1 | T-box transcription factor 1 |
| 56924 | PAK6 | p21 (RAC1) activated kinase 6 |
| 51162 | EGFL7 | EGF like domain multiple 7 |
| 642954 | RBBP4P1 | RBBP4 pseudogene 1 |
| 116154 | PHACTR3 | phosphatase and actin regulator 3 |
| 79838 | TMC5 | transmembrane channel like 5 |
| 1525 | CXADR | CXADR Ig-like cell adhesion molecule |
| 51286 | CEND1 | cell cycle exit and neuronal differentiation 1 |
| 767557 | SYS1-DBNDD2 | SYS1-DBNDD2 readthrough (NMD candidate) |
| 9249 | DHRS3 | dehydrogenase/reductase 3 |
| 8515 | ITGA10 | integrin subunit alpha 10 |
| 441425 | ANKRD20A3P | ankyrin repeat domain 20 family member A3, pseudogene |
| 793 | CALB1 | calbindin 1 |
| 202151 | RANBP3L | RAN binding protein 3 like |
| 84525 | HOPX | HOP homeobox |
| 7136 | TNNI2 | troponin I2, fast skeletal type |
| 4923 | NTSR1 | neurotensin receptor 1 |
| 90019 | SYT8 | synaptotagmin 8 |
| 3853 | KRT6A | keratin 6A |
| 196051 | PLPP4 | phospholipid phosphatase 4 |
| 2687 | GGT5 | gamma-glutamyltransferase 5 |
| 260293 | CYP4X1 | cytochrome P450 family 4 subfamily X member 1 |
| 286 | ANK1 | ankyrin 1 |
| 100533483 | DNAAF4-CCPG1 | DNAAF4-CCPG1 readthrough (NMD candidate) |
| 7062 | TCHH | trichohyalin |
| 85358 | SHANK3 | SH3 and multiple ankyrin repeat domains 3 |
| 8436 | CAVIN2 | caveolae associated protein 2 |
| 1464 | CSPG4 | chondroitin sulfate proteoglycan 4 |
| 94122 | SYTL5 | synaptotagmin like 5 |
| 3398 | ID2 | inhibitor of DNA binding 2 |
| 23508 | TTC9 | tetratricopeptide repeat domain 9 |
| 6755 | SSTR5 | somatostatin receptor 5 |
| 54873 | PALMD | palmdelphin |
| 3037 | HAS2 | hyaluronan synthase 2 |
| 494470 | RNF165 | ring finger protein 165 |
| 89874 | SLC25A21 | solute carrier family 25 member 21 |
| 55089 | SLC38A4 | solute carrier family 38 member 4 |
| 55638 | SYBU | syntabulin |
| 25891 | PAMR1 | peptidase domain containing associated with muscle regeneration 1 |
| 319085 | ITPK1-AS1 | ITPK1 antisense RNA 1 |
| 4586 | MUC5AC | mucin 5AC, oligomeric mucus/gel-forming |
| 51673 | TPPP3 | tubulin polymerization promoting protein family member 3 |
| 146802 | SLC47A2 | solute carrier family 47 member 2 |
| 3861 | KRT14 | keratin 14 |
| 191585 | PLAC4 | placenta enriched 4 |
| 1551 | CYP3A7 | cytochrome P450 family 3 subfamily A member 7 |
| 400916 | CHCHD10 | coiled-coil-helix-coiled-coil-helix domain containing 10 |
| 8645 | KCNK5 | potassium two pore domain channel subfamily K member 5 |
| 4128 | MAOA | monoamine oxidase A |
| 4856 | CCN3 | cellular communication network factor 3 |
| 3705 | ITPK1 | inositol-tetrakisphosphate 1-kinase |
| 2331 | FMOD | fibromodulin |
| 56241 | SUSD2 | sushi domain containing 2 |
| 56172 | ANKH | ANKH inorganic pyrophosphate transport regulator |
| 2624 | GATA2 | GATA binding protein 2 |
| 41 | ASIC1 | acid sensing ion channel subunit 1 |
| 79007 | DBNDD1 | dysbindin domain containing 1 |
| 5138 | PDE2A | phosphodiesterase 2A |
| 284021 | MILR1 | mast cell immunoglobulin like receptor 1 |
| 283897 | C16orf54 | chromosome 16 open reading frame 54 |
| 7045 | TGFBI | transforming growth factor beta induced |
| 6217 | RPS16 | ribosomal protein S16 |
| 1396 | CRIP1 | cysteine rich protein 1 |
| 353194 | KRT16P6 | keratin 16 pseudogene 6 |
| 5360 | PLTP | phospholipid transfer protein |
| 5187 | PER1 | period circadian regulator 1 |
| 90632 | LINC00473 | long intergenic non-protein coding RNA 473 |
| 4588 | MUC6 | mucin 6, oligomeric mucus/gel-forming |
| 6781 | STC1 | stanniocalcin 1 |
| 100505817 | LINC02582 | long intergenic non-protein coding RNA 2582 |
| 4680 | CEACAM6 | CEA cell adhesion molecule 6 |
| 2354 | FOSB | FosB proto-oncogene, AP-1 transcription factor subunit |
| 3776 | KCNK2 | potassium two pore domain channel subfamily K member 2 |
| 83643 | CCDC3 | coiled-coil domain containing 3 |
| 2696 | GIPR | gastric inhibitory polypeptide receptor |
| 3557 | IL1RN | interleukin 1 receptor antagonist |
| 2295 | FOXF2 | forkhead box F2 |
| 374378 | GALNT18 | polypeptide N-acetylgalactosaminyltransferase 18 |
| 2752 | GLUL | glutamate-ammonia ligase |
| 80195 | TMEM254 | transmembrane protein 254 |
| 6563 | SLC14A1 | solute carrier family 14 member 1 (Kidd blood group) |
| 93010 | B3GNT7 | UDP-GlcNAc:betaGal beta-1,3-N-acetylglucosaminyltransferase 7 |
| 145482 | PTGR2 | prostaglandin reductase 2 |
| 6564 | SLC15A1 | solute carrier family 15 member 1 |
| 5583 | PRKCH | protein kinase C eta |
| 112399 | EGLN3 | egl-9 family hypoxia inducible factor 3 |
| 55107 | ANO1 | anoctamin 1 |
| 627 | BDNF | brain derived neurotrophic factor |
| 875 | CBS | cystathionine beta-synthase |
| 6337 | SCNN1A | sodium channel epithelial 1 subunit alpha |
| 23242 | COBL | cordon-bleu WH2 repeat protein |
| 56103 | PCDHGB2 | protocadherin gamma subfamily B, 2 |
| 1645 | AKR1C1 | aldo-keto reductase family 1 member C1 |
| 1646 | AKR1C2 | aldo-keto reductase family 1 member C2 |
| 286343 | LURAP1L | leucine rich adaptor protein 1 like |
| 1397 | CRIP2 | cysteine rich protein 2 |
| 64115 | VSIR | V-set immunoregulatory receptor |
| 3934 | LCN2 | lipocalin 2 |
| 1293 | COL6A3 | collagen type VI alpha 3 chain |
| 85379 | KIAA1671 | KIAA1671 |
| 9351 | SLC9A3R2 | SLC9A3 regulator 2 |
| 9252 | RPS6KA5 | ribosomal protein S6 kinase A5 |
| 23213 | SULF1 | sulfatase 1 |
| 3958 | LGALS3 | galectin 3 |

TABLE 2
Full list of DDR2-knockdown related genes:

| Entrez | | |
|---|---|---|
| Gene ID | Symbol | Description |
| 225642 | Grp | gastrin releasing peptide |
| 11656 | Alas2 | aminolevulinic acid synthase 2, erythroid |
| 15122 | Hba-a1 | hemoglobin alpha, adult chain 1 |
| 217328 | Myo15b | myosin XVB |
| 12985 | Csf3 | colony stimulating factor 3 (granulocyte) |
| 110257 | Hba-a2 | hemoglobin alpha, adult chain 2 |
| 16165 | Il13ra2 | interleukin 13 receptor, alpha 2 |
| 11687 | Alox15 | arachidonate 15-lipoxygenase |
| 11684 | Alox12 | arachidonate 12-lipoxygenase |
| 20311 | Cxcl5 | chemokine (C-X-C motif) ligand 5 |
| 16017 | Ighg1 | immunoglobulin heavy constant gamma 1 (G1m marker) |
| 217733 | Tmem63c | transmembrane protein 63c |
| 20310 | Cxcl2 | chemokine (C-X-C motif) ligand 2 |
| 546144 | Wdr72 | WD repeat domain 72 |
| 228564 | Frmd5 | FERM domain containing 5 |
| 14917 | Gucy2c | guanylate cyclase 2c |
| 26878 | B3galt2 | UDP-Gal:betaGlcNAc beta 1,3-galactosyltransferase, polypeptide 2 |
| 216643 | Gabrp | gamma-aminobutync acid (GABA) A receptor, pi |
| 19016 | Pparg | peroxisome proliferator activated receptor gamma |
| 330627 | Trim66 | tripartite motif-containing 66 |
| 100727 | Ugt2b34 | UDP glucuronosyltransferase 2 family, polypeptide B34 |
| 15112 | Hao1 | hydroxyacid oxidase 1, liver |
| 21990 | Tph1 | tryptophan hydroxylase 1 |
| 20202 | S100a9 | S100 calcium binding protein A9 (calgranulin B) |
| 330122 | Cxcl3 | chemokine (C-X-C motif) ligand 3 |
| 140919 | Slc17a6 | solute carrier family 17 (sodium-dependent inorganic phosphate cotransporter) |
| | | member 6 |
| 12981 | Csf2 | colony stimulating factor 2 (granulocyte-macrophage) |
| 11571 | Crisp1 | cysteine-rich secretory protein 1 |
| 381651 | Odaph | odontogenesis associated phosphoprotein |
| 11685 | Alox12e | arachidonate lipoxygenase, epidermal |
| 70918 | Nsun7 | NOL1/NOP2/Sun domain family, member 7 |
| 16691 | Krt8 | keratin 8 |
| 53897 | Gal3st1 | galactose-3-O-sulfotransferase 1 |
| 72077 | Gcnt3 | glucosaminyl (N-acetyl) transferase 3, mucin type |
| 16175 | Il1a | interleukin 1 alpha |
| 56221 | Ccl24 | chemokine (C-C motif) ligand 24 |
| 226610 | Fam78b | family with sequence similarity 78, member B |
| 628324 | S100a2 | S100 calcium binding protein A2 |
| 18703 | Pigr | polymeric immunoglobulin receptor |
| 53624 | Cldn7 | claudin 7 |
| 20297 | Ccl20 | chemokine (C-C motif) ligand 20 |
| 17384 | Mmp10 | matrix metallopeptidase 10 |
| 14600 | Ghr | growth hormone receptor |
| 18413 | Osm | oncostatin M |
| 21946 | Pglyrp1 | peptidoglycan recognition protein 1 |
| 22249 | Unc13b | unc-13 homolog B |
| 20755 | Sprr2a1 | small proline-rich protein 2A1 |
| 12351 | Car4 | carbonic anhydrase 4 |
| 114142 | Foxp2 | forkhead box P2 |
| 17002 | Ltf | lactotransferrin |
| 17829 | Muc1 | mucin 1, transmembrane |
| 14825 | Cxcl1 | chemokine (C-X-C motif) ligand 1 |
| 14133 | Fcna | ficolin A |
| 57349 | Ppbp | pro-platelet basic protein |
| 70894 | Efcab3 | EF-hand calcium binding domain 3 |
| 110310 | Krt7 | keratin 7 |
| 69993 | Chn2 | chimerin 2 |
| 70350 | Basp1 | brain abundant, membrane attached signal protein 1 |
| 93961 | B3galt5 | UDP-Gal:betaGlcNAc beta 1,3-galactosyltransferase, polypeptide 5 |
| 22317 | Vamp1 | vesicle-associated membrane protein 1 |
| 78906 | Misp | mitotic spindle positioning |
| 66889 | Rnf128 | ring finger protein 128 |
| 213948 | Atg9b | autophagy related 9B |
| 72269 | Cda | cytidine deaminase |
| 13522 | Adam28 | a disintegrin and metallopeptidase domain 28 |
| 70835 | Prss22 | protease, serine 22 |
| 56312 | Nupr1 | nuclear protein transcription regulator 1 |
| 16669 | Krt19 | keratin 19 |
| 20201 | S100a8 | S100 calcium binding protein A8 (calgranulin A) |

TABLE 3
DDR1-overexpression related genes correlated with
high overall survival in the Z-score model.

| Entrez Gene ID | Symbol | log₂FC (CR vs. PD) |
|---|---|---|
| 360 | AQP3 | −0.80045 |
| 56901 | NDUFA4L2 | −0.52419 |
| 5064 | PALM | −0.30714 |
| 9249 | DHRS3 | −0.39227 |
| 2687 | GGT5 | −0.41081 |
| 2696 | GIPR | −0.58305 |
| 374378 | GALNT18 | −0.36309 |
| 55107 | ANO1 | −0.43583 |
| 56103 | PCDHGB2 | −0.41683 |
| 286343 | LURAP1L | −0.68979 |

TABLE 4
DDR1-overexpression related genes correlated
with high overall survival in the Cox model.

| Entrez Gene ID | Symbol | log₂FC (CR vs. PD) |
|---|---|---|
| 7031 | TFF1 | −0.90075 |
| 11199 | ANXA10 | −2.1676 |
| 8857 | FCGBP | −0.90073 |
| 90865 | IL33 | −0.35132 |
| 10529 | NEBL | 0.356101 |
| 9537 | TP53I11 | −0.39781 |
| 120224 | TMEM45B | −0.58108 |
| 90523 | MLIP | 0.187131 |
| 10863 | ADAM28 | −0.06444 |
| 114784 | CSMD2 | 0.082547 |
| 1388 | ATF6B | −0.05956 |
| 56901 | NDUFA4L2 | −0.52419 |
| 11247 | NXPH4 | 0.212618 |
| 388743 | CAPN8 | −1.04855 |
| 256158 | HMCN2 | −0.21974 |
| 6338 | SCNN1B | 0.6252 |
| 218 | ALDH3A1 | −0.16768 |
| 374918 | IGFL1 | 0.843856 |
| 1672 | DEFB1 | 0.01582 |

TABLE 5
DDR2-knockdown related genes correlated with
high overall survival in the Z-score model.

| Entrez Gene ID | Symbol | log₂FC (CR vs. PD) |
|---|---|---|
| 6273 | S100A2 | −1.73606 |
| 9245 | GCNT3 | −0.71333 |
| 6372 | CXCL6 | −0.66467 |
| 4319 | MMP10 | −2.14264 |

TABLE 6
DDR2-knockdown related genes correlated with
high overall survival in the Cox model.

| Entrez Gene ID | Symbol | log₂FC (CR vs. PD) |
|---|---|---|
| 2922 | GRP | −0.28229 |
| 212 | ALAS2 | −0.75455 |
| 3040 | HBA2 | −0.37202 |
| 80022 | MYO15B | −0.29427 |
| 3039 | HBA1 | −0.50559 |
| 3598 | IL13RA2 | 0.301616 |
| 246 | ALOX15 | −0.36571 |
| 239 | ALOX12 | 0.964897 |
| 6372 | CXCL6 | −0.66467 |
| 57156 | TMEM63C | 0.241303 |
| 2920 | CXCL2 | 0.080034 |
| 256764 | WDR72 | 0.113391 |
| 84978 | FRMD5 | −0.08131 |
| 2984 | GUCY2C | 0.133768 |
| 8707 | B3GALT2 | 0.292314 |
| 2568 | GABRP | −0.60858 |
| 5468 | PPARG | −0.09445 |
| 9866 | TRIM66 | 0.00296 |
| 7166 | TPH1 | 0.230412 |
| 6280 | S100A9 | 0.4511 |
| 2921 | CXCL3 | −0.05529 |
| 1437 | CSF2 | −0.25575 |
| 10321 | CRISP3 | −0.16152 |
| 152816 | ODAPH | 0.204385 |
| 79730 | NSUN7 | 0.066317 |

TABLE 7
Union of DDR1-overexpression related genes (table 3)
and DDR2-knockdown related genes (table 5) correlated
with high overall survival in the Z-score model.

| | log₂FC | |
|---|---|---|
| Symbol | (CR vs. PD) | Name of Genes or Encoded Proteins |
| AQP3 | −0.80045 | Aquaporin 3 (Gill Blood Group) |
| NDUFA4L2 | −0.52419 | NADH dehydrogenase 1 alpha subcomplex, |
| | | 4-like 2 |
| PALM | −0.30714 | Paralemmin |
| DHRS3 | −0.39227 | Dehydrogenase/Reductase 3 |
| GGT5 | −0.41081 | Gamma-Glutamyltransferase 5 |
| GIPR | −0.58305 | Gastric Inhibitory Polypeptide Receptor |
| GALNT18 | −0.36309 | Polypeptide N- |
| | | Acetylgalactosaminyltransferase 18 |
| ANO1 | −0.43583 | Anoctamin-1 |
| PCDHGB2 | −0.41683 | Protocadherin Gamma Subfamily B, 2 |
| LURAP1L | −0.68979 | Leucine Rich Adaptor Protein 1 Like |
| S100A2 | −1.73606 | S100 Calcium Binding Protein A2 |
| GCNT3 | −0.71333 | Glucosammyl (N-Acetyl) Transferase 3 |
| CXCL6 | −0.66467 | C-X-C Motif Chemokine Ligand 6 |
| MMP10 | −2.14264 | matrix metalloproteinase-10 |

TABLE 8
Union of DDR1-overexpression related genes (table 4) and DDR2-knockdown related
genes (table 6) correlated with high overall survival in the Cox model.

| EntrezID | Symbol | log₂FC (CR vs. PD) | Name of Genes of Encoded Proteins |
|---|---|---|---|
| 56901 | NDUFA4L2 | −0.52419 | NADH dehydrogenase 1 alpha subcomplex, 4-like 2 |
| 80022 | MYO15B | −0.29427 | myosin XVB |
| 90523 | MLIP | 0.187131 | Muscular LMNA Interacting Protein |
| 6372 | CXCL6 | −0.66467 | Chemokine (C-X-C motif) ligand 6 |
| 256764 | WDR72 | 0.113391 | WD repeat-containing protein 72 |
| 2921 | CXCL3 | −0.05529 | Chemokine (C-X-C motif) ligand 3 |
| 8857 | FCGBP | −0.90073 | IgGFc-binding protein |
| 114784 | CSMD2 | 0.082547 | CUB and sushi domain-containing protein 2 |
| 2920 | CXCL2 | 0.080034 | Chemokine (C-X-C motif) ligand 2 |
| 7166 | TPH1 | 0.230412 | Tryptophan hydroxylase 1 |
| 1672 | DEFB1 | 0.01582 | Beta-defensin 1 |
| 7031 | TFF1 | −0.90075 | Trefoil Factor 1 |
| 9866 | TRIM66 | 0.00296 | Tripartite Motif Containing 66 |
| 218 | ALDH3A1 | −0.16768 | Aldehyde Dehydrogenase 3 Family Member A1 |
| 11199 | ANXA10 | −2.1676 | Annexin A10 |
| 1437 | CSF2 | −0.25575 | Colony Stimulating Factor 2 |
| 90865 | IL33 | −0.35132 | Interleukin 33 |
| 9537 | TP53I11 | −0.39781 | Tumor Protein P53 Inducible Protein 11 |
| 388743 | CAPN8 | −1.04855 | Calpain 8 |
| 212 | ALAS2 | −0.75455 | Delta-aminolevulinate synthase 2 |
| 11247 | NXPH4 | 0.212618 | Neurexophilin 4 |
| 84978 | FRMD5 | −0.08131 | FERM Domain Containing 5 |
| 6280 | S100A9 | 0.4511 | S100 calcium-binding protein A9 |
| 57156 | TMEM63C | 0.241303 | Transmembrane Protein 63C |
| 2984 | GUCY2C | 0.133768 | guanylate cyclase 2c |
| 120224 | TMEM45B | −0.58108 | Transmembrane Protein 45B |
| 2922 | GRP | −0.28229 | Gastrin Releasing Peptide |
| 10863 | ADAM28 | −0.06444 | Disintegrin and metalloproteinase domain- |
| | | | containing protein 28 |
| 2568 | GABRP | −0.60858 | Gamma-aminobutyric acid receptor subunit pi |
| 239 | ALOX12 | 0.964897 | arachidonate 12-lipoxygenase |
| 10529 | NEBL | 0.356101 | Nebulette |
| 3039 | HBA1 | −0.50559 | hemoglobin subunit alpha 1 |
| 374918 | IGFL1 | 0.843856 | Insulin-like growth factor 1 |
| 6338 | SCNN1B | 0.6252 | sodium channel epithelial 1 subunit beta |
| 3040 | HBA2 | −0.37202 | Hemoglobin, alpha 2 |
| 256158 | HMCN2 | −0.21974 | Hemicentin 2 |
| 3598 | IL13RA2 | 0.301616 | Interleukin 13 Receptor Subunit Alpha 2 |
| 5468 | PPARG | −0.09445 | Peroxisome proliferator-activated receptor γ |
| 152816 | ODAPH | 0.204385 | Odontogenesis Associated Phosphoprotein |
| 10321 | CRISP3 | −0.16152 | Cysteine Rich Secretory Protein 3 |
| 246 | ALOX15 | −0.36571 | Arachidonate 15-Lipoxygenase |
| 8707 | B3GALT2 | 0.292314 | Beta-1,3-Galactosyltransferase 2 |
| 79730 | NSUN7 | 0.066317 | NOP2/Sun RNA Methyltransferase Family |
| | | | Member 7 |
| 1388 | ATF6B | −0.05956 | Activating Transcription Factor 6 Beta |

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1. Discoidin Domain Receptor-Driven Gene Signatures Stratify Patient Response to Anti PD-L1 Immune Checkpoint Therapy

Brief summary: Anti PD-1/PD-L1 based immune checkpoint therapy (ICT) provide durable responses in many patients with various cancer types and has emerged as one of the major pillars in anti-cancer therapies. However, a significant fraction of patients does not respond, and research is needed to identify biomarkers for their stratification to alternative treatments or clinical trials. Recently, we implicated discoidin domain receptor tyrosine kinase 2 (DDR2), a major collagen receptor, as a contributor to anti-PD-1 resistance. Here, we investigate whether downstream genes regulated by DDR2 and its closely related family member DDR1, might be able to stratify response to ICT in patients with cancer. Given that some patients with advanced bladder cancer exhibit durable responses to anti PD-1/PD-L1 ICT, we use this tumor type to evaluate this idea. Interestingly, DDR1 and DDR2 showed mutually exclusive expression pattern in human tumor tissues. In cancers with high DDR2 gene expression, gene set enrichment analysis revealed the enrichment of immune pathways, as well as a high “immune score”, indicative of a T-cell-inflamed phenotype. Conversely, bladder cancers with high DDR1 gene expression exhibit a non-T-cell-inflamed phenotype. Further, transcriptome analysis was conducted using bladder cancer models in which both DDR receptor tyrosine kinases (RTKs) were perturbed, and the corresponding DDR1- and DDR2-driven signature scores were compiled. The utility of these signature scores as a prognostic tool to stratify anti PD-L1 patient response was evaluated first on tumor data from the IMvigor210 bladder cancer clinical trial and then from that in several independent non-small cell lung cancer cohorts. Remarkably, DDR1 and 2 signature scores were able to successfully stratify response to ICT, likely reflecting their unique biology in modulating immune checkpoint response.

### 1.1 Divergent Expression of Discoidin Domain Receptor 1 and 2 in Bladder Carcinomas

We previously reported the biologic roles of the discoidin domain receptors (DDR) family, DDR1 and DDR2, in modulating bladder cancer metastasis and immune checkpoint therapy response, respectively. Here, we perform a comprehensive evaluation of this DDR gene family using the BLCA TCGA dataset (Table 9) (n=259).

TABLE 9
Clinicopathological characteristics of TCGA BLCA
cohort by DDR expression levels (n = 259*)

| | | Level of DDR1 and DDR2 Gene Expression | | | |
|---|---|---|---|---|---|
| | | DDR1 | DDR1 | DDR1 | DDR1 |
| | | high & | high & | low & | low & |
| | | DDR2 | DDR2 | DDR2 | DDR2 |
| Characteristics | | High | low | high | low |
| Sex | Female | 10 | 22 | 26 | 12 |
| | Male | 39 | 58 | 54 | 38 |
| Race | Caucasian | 47 | 62 | 69 | 38 |
| | African | 0 | 4 | 5 | 4 |
| | American | | | | |
| | Asian | 1 | 9 | 4 | 7 |
| | N/A | 1 | 5 | 2 | 1 |
| Pathologic | II | 10 | 34 | 12 | 12 |
| stage | III | 17 | 27 | 37 | 24 |
| | IV | 22 | 19 | 31 | 14 |
| T stage | T2 | 11 | 39 | 13 | 14 |
| | T3 | 28 | 33 | 54 | 28 |
| | T4 | 10 | 8 | 13 | 8 |
| Recurrent | No | 17 | 40 | 28 | 18 |
| | Recurrent | 2 | 10 | 5 | 5 |
| | N/A | 30 | 30 | 47 | 27 |
| Overall | Living | 24 | 53 | 40 | 29 |
| survival | Deceased | 25 | 27 | 40 | 29 |
| Histological | High grade | 49 | 80 | 80 | 50 |
| grade | Low grade | 0 | 0 | 0 | 0 |

*Out of a total 407 samples, 259 samples were selected by filtering with these criteria: 1) any stage other than T2-4; 2) 52 (13%) had urothelial carcinoma with variant histology, including 42 squamous, 4 small cell/neuroendocrine, 2 micropapillary, and 4 plasmacytoid. 5 additional tumors that met screening criteria were included: 3 pure squamous cell bladder carcinomas, 1 squamous cell carcinoma of non-bladder origin, and 1 bladder adenocarcinoma. So total of 57; 3) 35 patients had received prior intravesical immunotherapy with Bacille Calmette-Guerin (BCG); and 4) 12 had received neoadjuvant chemotherapy (NAC).

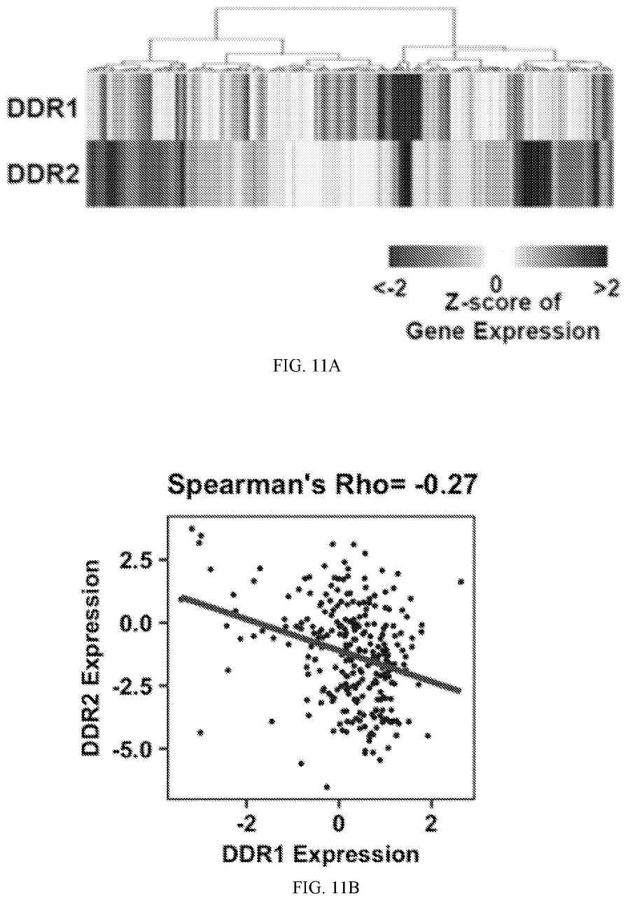

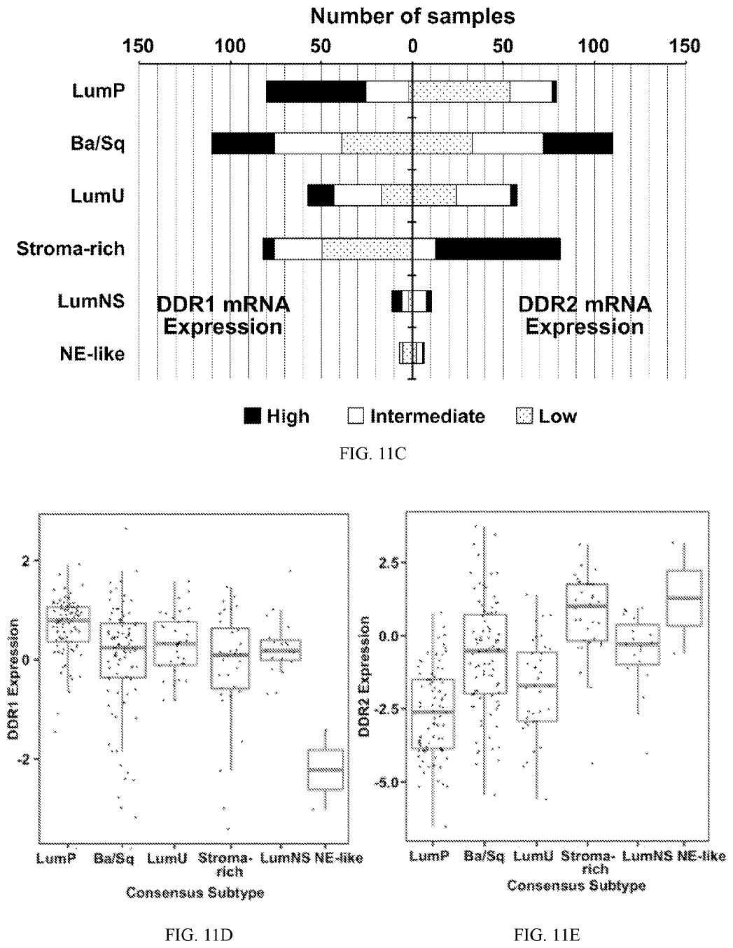

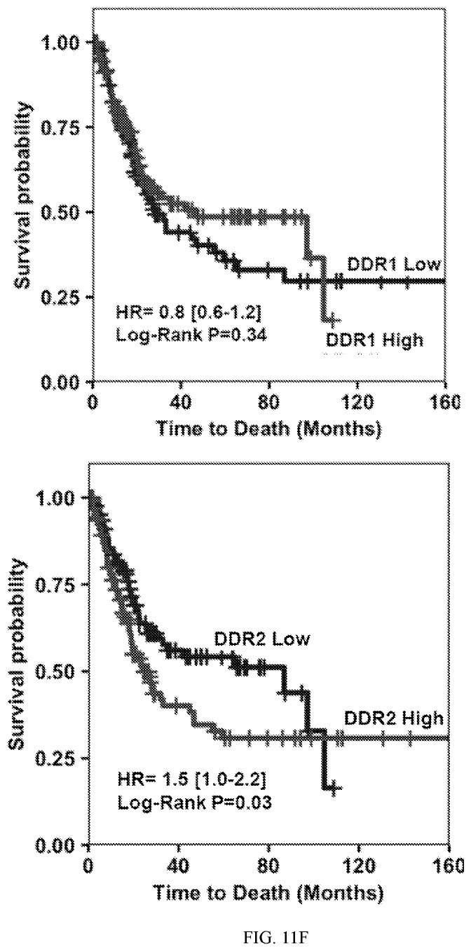

Unsupervised hierarchical clustering analysis of DDR1 and DDR2 expression shows clear separation of DDR1^{high}; DDR2^{low} versus DDR1^{low}; DDR2^{high} tumors into two groups (FIG. 11A; Chi-square P-value=0.0002). Further analysis revealed DDR2 demonstrates an inverse linear relationship to that of DDR1 expression (FIG. 11B, Spearman's ρ=−0.27). Taken together, these findings indicate DDR1 and DDR2 gene expression may reflect bladder carcinomas with distinct characteristics.

Since gene expression defined bladder cancer subtypes have been associated with different clinical behaviors in patients, we investigated the distribution and prevalence of high DDR1 or DDR2 tumor expression (DDR1^{high} and DDR2^{high}) among various bladder cancer subtypes, namely Luminal papillary (LumP), Basal/squamous (Ba/Sq), Luminal unstable (LumU), Stromal-rich, Luminal non-specified (LumNS), and Neuroendocrine-like (NE-like) (FIG. 11C). Interestingly, LumP and Ba/Sq subtypes which are reported to have divergent properties both have a relatively higher fraction and overall levels of DDR1^{high} expression (FIG. 11C, 11D; Chi-square P-value=0.0024). In contrast, only the Ba/Sq subtype has a relatively high proportion of DDR2^{high} tumors (FIG. 11C, 11E). Furthermore, a high proportion of DDR2^{high} tumors is also found in the Stromal-rich subtype. Such finding is interesting, since DDR2 is not restrictedly expressed in epithelial cancer cells, but is also found to be expressed in stromal fibroblasts in several cancer types. Thus, contribution of DDR2 expression in this Stromal-rich subtype might come from cancer associated fibroblasts as well as epithelial cells.

The above findings suggest that the commonly defined bladder cancer subtypes are more diverse than previously appreciated and that such diversity and its clinical consequences may in part be driven by DDR1 or DDR2 expression. In addition, it supports a role for stromal host components in bladder cancer behavior. Given this, we examined if mRNA expression of DDR1 or DDR2 alone stratified outcome in the TCGA patient cohort. While DDR1^{high} tumors trended toward a better overall survival this was not significant (p=0.34) (FIG. 11F). In contrast, DDR2^{high} tumors were associated with a poor overall survival (p=0.03) (FIG. 11F) suggesting that DDR2 may be a driver of tumor growth and progression in human bladder cancer.

1.2 Distinct Bladder Tumor Microenvironment (bTME) in DDR1^{high} and DDR2^{high} Tumors

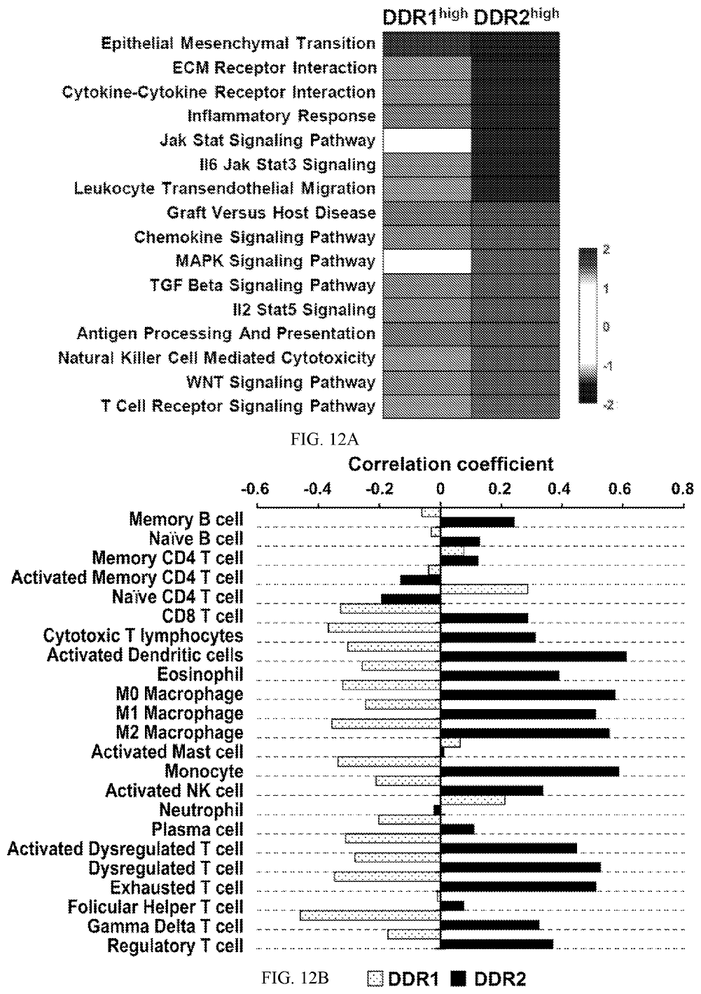

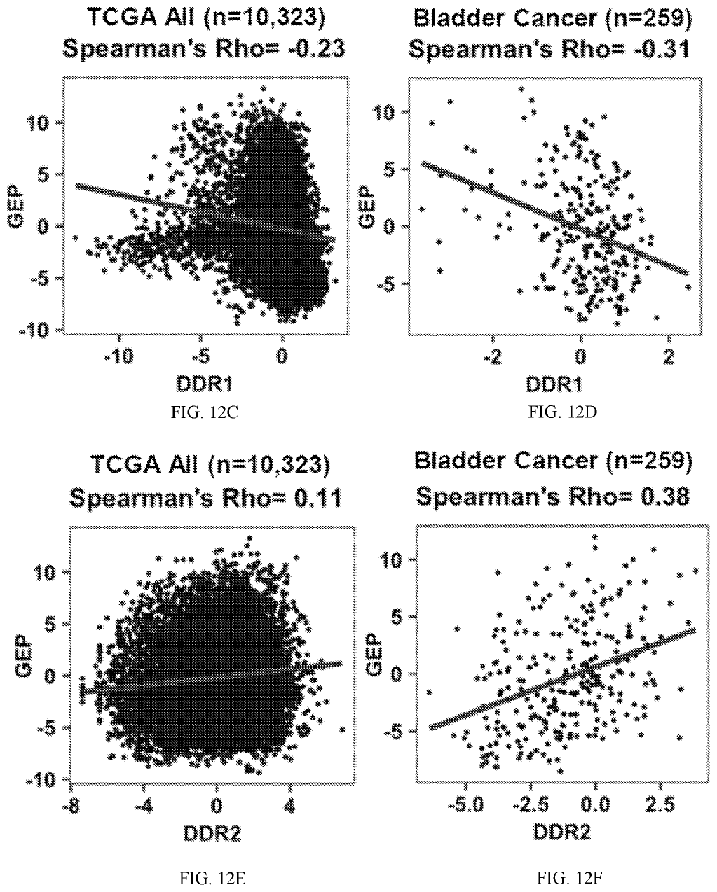

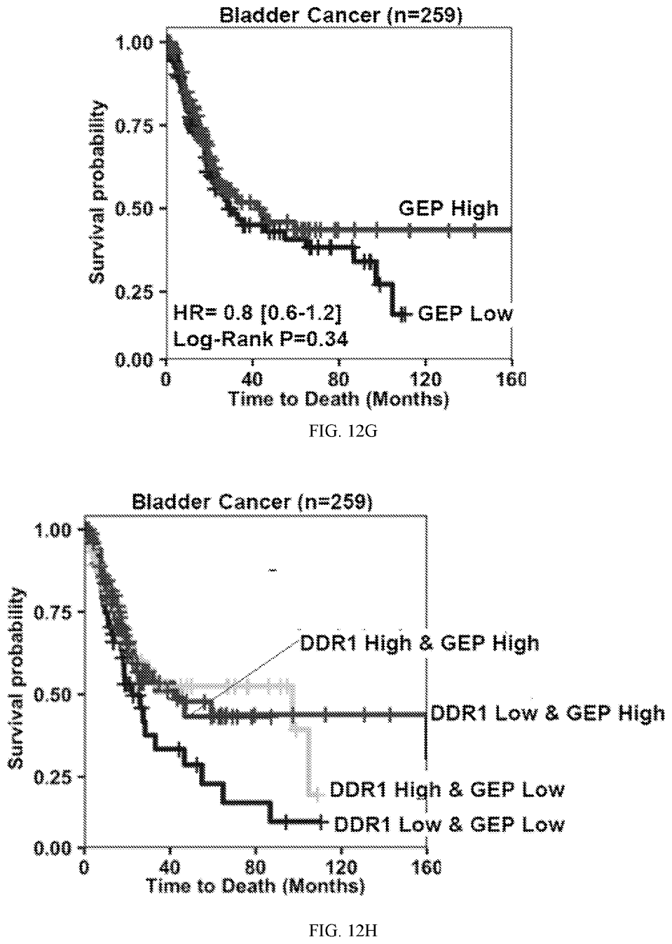

To further investigate the biological consequences of DDR1^{high} and DDR2^{high} in bladder carcinomas, we performed gene set enrichment analysis (GSEA) and digital dissection analyses to identify the cellular processes and immune cell components that are enriched in each of these tumors. We first stratified TCGA BC tumors into DDR1 (or DDR2) high and low at their median expression level and then compared the high and low groups for ranking genes ascending order. Median expression level of DDR1 (or DDR2) was computed with expression values across 259 TCGA BC tumor samples. When the sample has higher DDR1 (or DDR2) expression compared to the median value of DDR1 (or DDR2), it assigned to DDR1^{high} (or DDR2^{high}) group. Finally, normalized enrichment score of 50 hallmark gene sets were computed for DDR1 and DDR2, separately. Of note, 16 hallmark gene sets are significantly enriched (GSEA nominal P<0.05) in DDR2^{high} tumors compared to DDR2^{low} tumors, while these gene sets exhibit low expression in DDR1^{high} tumors compared to DDR1^{low} tumors (FIG. 12A). Intriguingly, DDR2^{high} bladder carcinomas are highly enriched in cellular processes such as “Epithelial Mesenchymal Transition” and “ExtraCellular Matrix (ECM) receptor interaction” (FIG. 12A), supporting its higher prevalence in the Stroma-rich subtype (FIG. 11C). On the other hand, DDR1^{high} bladder carcinomas are primarily enriched in the “TGFB signaling pathway” and “WNT signaling pathway” (FIG. 12A). Intriguingly, another key finding is that DDR2^{high} tumors are highly enriched with a spectrum of cellular processes related to immune cells, including “Cytokine-Cytokine receptor interaction”, “Inflammatory response”, “Chemokine signaling pathway”, “Antigen processing and presentation”, “Natural killer cell mediated cytotoxicity” and “T cell receptor signaling pathway” (FIG. 12A), indicative of its role in immune regulation and suggesting an association of DDR2 expression with a T-cell-inflamed or “hot” phenotype. Conversely, DDR1^{high} tumors are negatively associated with these immune cell-related cellular processes (FIG. 12A), indicating such tumors are more likely to be non-T-cell-inflamed or “cold”. Relevant genes in FIG. 12A and FIG. 12B are defined by Gene Ontology Biological Processes and can be viewed in Gene Ontology database.

To evaluate these findings in more detail, we performed digital dissection via transcriptome-based cell type quantification method using MCP-Counter to estimate how the presence of various immune cell types correlates with DDR expression (FIG. 12B). We assessed whether immune infiltrates are correlated with DDR1 or DDR2 expression levels using 23 immune cell type signatures, which were obtained from three tools including CIBERSORT, MCP-counter and xCell and refined the signatures with unique genes for each cell types (i.e., memory B cells, naïve B cells, memory CD4 T cells, activated memory CD4 T cells, naïve CD4 T cells, CD8 T cells, cytotoxic T lymphocytes, activated dendritic cells, eosinophils, M0 macrophages, M1 macrophages, M2 macrophages, activated mast cells, monocytes, activated NK cells, neutrophils, plasma cells, activated dysregulated T cells, dysregulated T cells, exhausted T cells, follicular helper T cells, gamma delta T cells, and regulatory T cells). Consistent with the GSEA analysis in FIG. 12A, the correlation coefficients of immune cell types are primarily opposite between DDR1^{high} and DDR2^{high} bladder carcinomas (FIG. 12B). DDR2^{high} tumors are positively correlated with a wide spectrum of immune cells, spanning B cells, T cells, dendritic cells, macrophages, monocytes and NK cells (FIG. 12B), indicative of an immunologically “hot” bladder tumor microenvironment (bTME). Conversely, DDR1^{high} tumors are negatively correlated with all immune cell types analyzed, except memory and naïve CD4+ T cells, mast cells and neutrophils (FIG. 12B), indicative of an immunologically “cold” bTME. These analyses for the first time highlight a completely distinct bladder tumor microenvironment (bTME) in DDR1^{high} and DDR2^{high} bladder carcinomas supporting the notion these genes have different biological roles. This is particularly unexpected given their functional similarity.

Next, we sought to determine if the relationship between DDR1 and DDR2 gene expression and the immune TME in bladder cancer is also present across various tumor types. T cell inflamed gene-expression profile (GEP) score was used as an estimation for assessing the immune TME, and its correlation with DDR1 and DDR2 gene expression evaluated using the pan TCGA dataset (n=10,323) (with transcriptomic data obtained from Xena browser). T cell inflamed GEP signature contains 18 genes including CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, TIGIT. Weighted Z-score method was used to compute the T cell inflamed GEP score of individual tumor samples. Patients were stratified by DDR1 and DDR2 gene expression levels and T cell inflamed GEP score (median) using cutoffs equivalent in terms of prevalence to those that were used to define the clinical response groups in the pan-cancer cohort. Consistent with the digital dissection data described above indicating DDR1^{high} tumors are immunologically “cold” (FIG. 12B), DDR1 gene expression is inversely correlated with T cell inflamed GEP score in both pan-cancer TCGA (FIG. 12C, ρ=−0.23 p<0.001) and bladder TCGA cohorts (FIG. 12D, ρ=−0.31, p<0.001). Conversely, DDR2 gene expression is significantly correlated with the T cell inflamed GEP score in both pan-cancer TCGA (FIG. 12E, ρ=0.11; p<0.001) and bladder TCGA cohorts (FIG. 12F, ρ=0.38; p<0.001), as did DDR2 and CD8 T cell (r=0.29; p<0.001) and DDR2 and Cytotoxic T lymphocytes (r-0.31; p<0.001) (FIG. 12B), supporting the notion that DDR2^{high} tumors are immunologically “hot”.

TABLE 10
Multiple Log-Rank tests were performed with DDR^{low} &
GEP^{high} group in TCGA BLCA cohort as a base line. Table
shows Hazard ratio (HR), significance level (P-value)
and confidence interval (CI) for each comparison.

| Comparison | HR | P-value | CI 95% |
|---|---|---|---|
| DDR1 Hi & GEP Low vs. | 0.58 | 0.033 | 0.35-0.95 |
| DDR1 Low & GEP Low | | | |
| DDR1 Low & GEP Hi vs. | 0.56 | 0.026 | 0.34-0.93 |
| DDR1 Low & GEP Low | | | |
| DDR1 Hi & GEP Hi vs. | 0.64 | 0.107 | 0.37-1.10 |
| DDR1 Low & GEP How | | | |
| DDR2 Low & GEP Low vs. | 1.74 | 0.089 | 0.92-3.28 |
| DDR2 Low & GEP High | | | |
| DDR2 Hi & GEP Low vs. | 2.51 | 0.007 | 1.28-4.91 |
| DDR2 Low & GEP High | | | |
| DDR2 Hi & GEP Hi vs. | 2.02 | 0.028 | 1.08-3.79 |
| DDR2 Low & GEP High | | | |

Survival analysis was also performed using bladder TCGA cohort (FIG. 12G-12I). GEP score alone has no significance association with overall survival in the cohort (FIG. 12G), while further stratified GEP scores by DDR1 and DDR2 gene expression exhibits differences between patient subgroups. DDR1^{low} & GEP^{low} group shows significant poor overall survival compared to DDR1^{hi} & GEP^{low} and DDR1^{low} & GEP^{hi} groups (FIG. 12H). Additionally, DDR2 low groups (i.e. DDR2^{low} & GEP^{low}, DDR2^{low} & GEP^{hi}) exhibit better overall survival compared to DDR2 high groups (i.e. DDR2^{hi} & GEP^{low} and DDR2^{hi} & GEP^{hi}) (FIG. 12I). This implies that DDR and GEP are associated with patient survival with bladder cancer.

### 1.3 Association of DDR Expression and Response to Immune Checkpoint Therapy

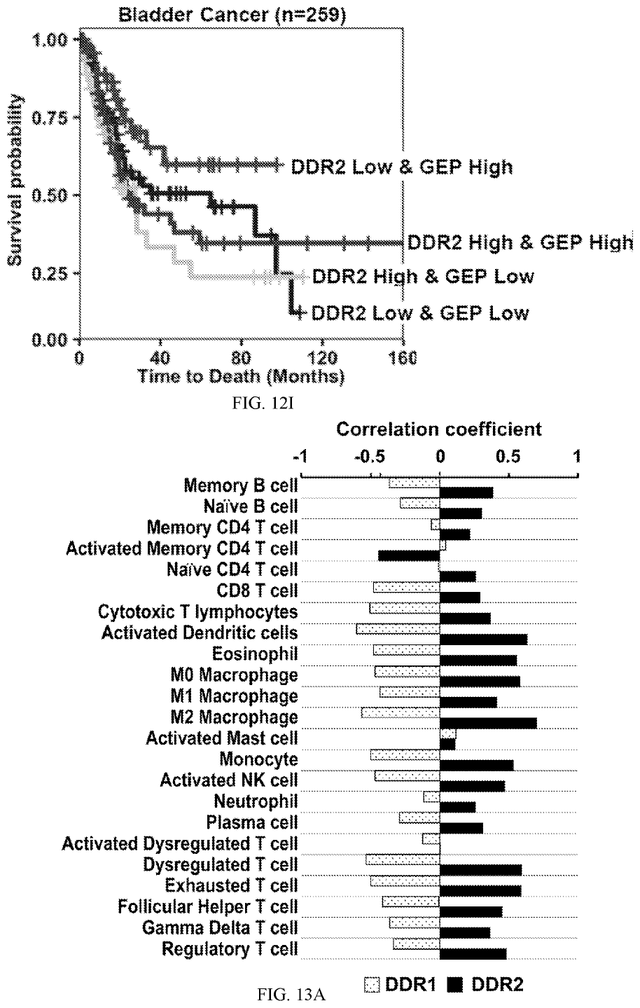

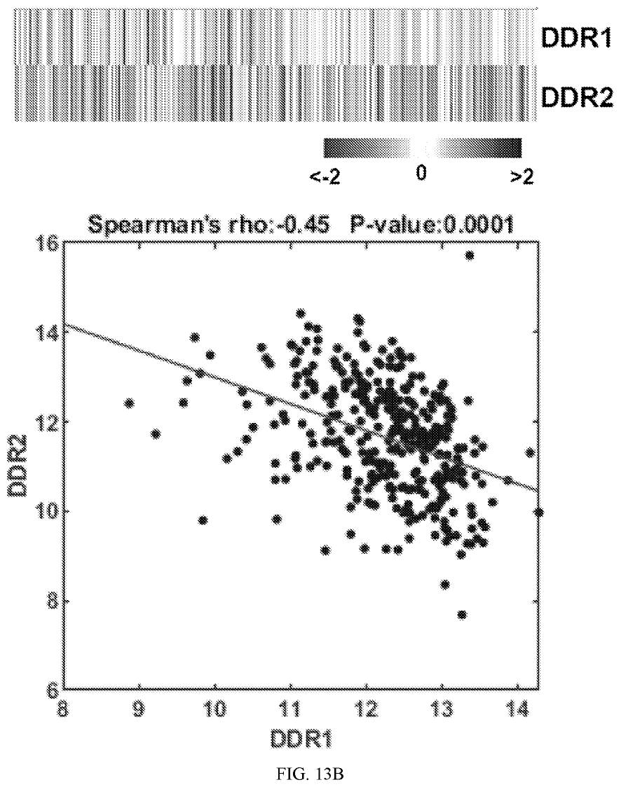

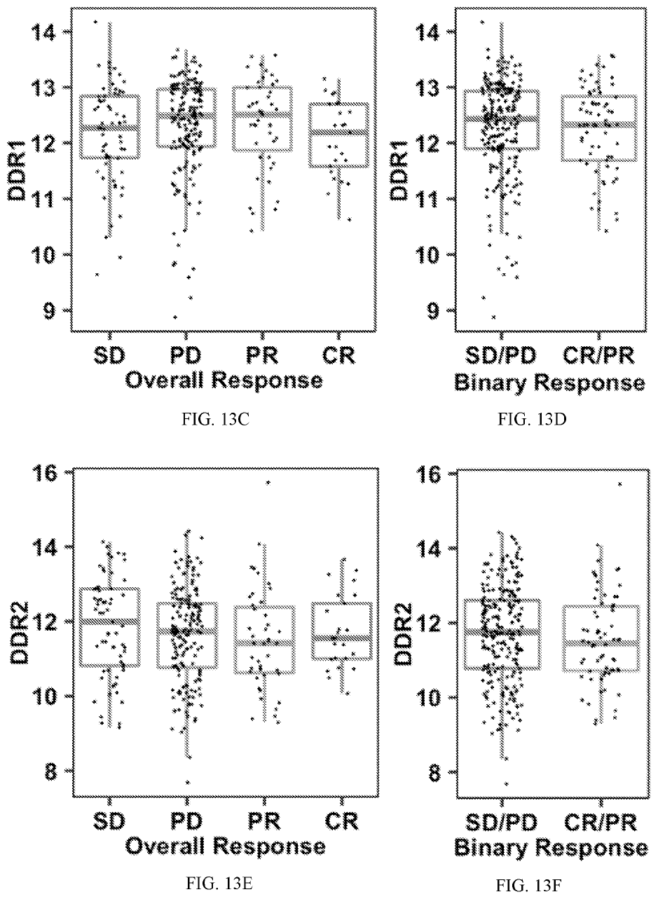

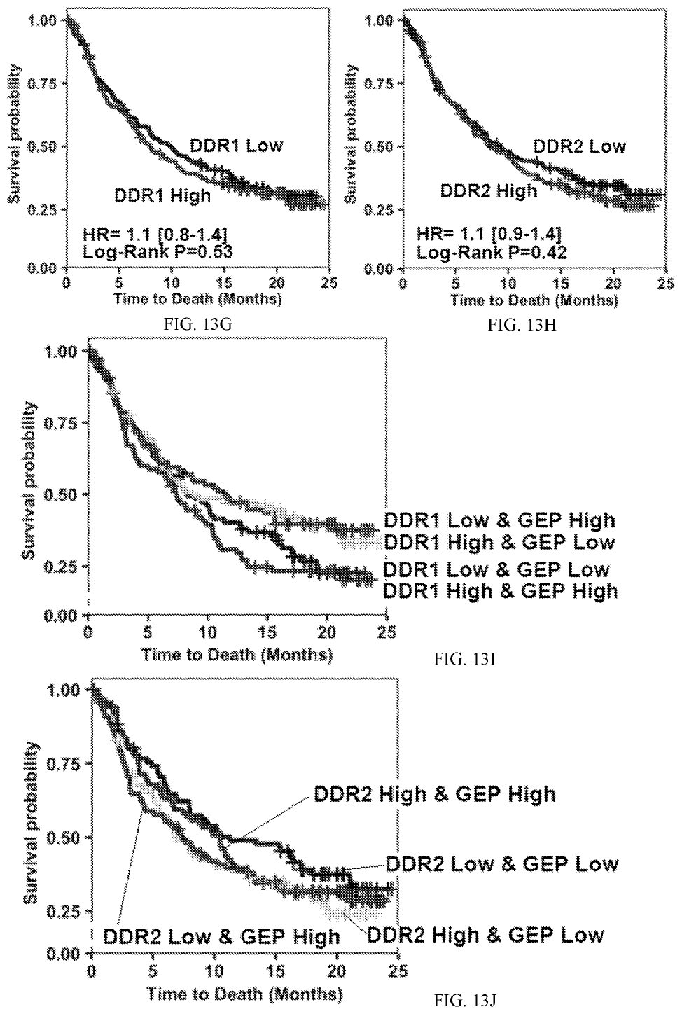

DDR1^{high} and DDR2^{high} tumors appear to be associated with two divergent TMEs, immunologically “cold” and “hot” respectively and in the case DDR2^{high} portend for a worse patient outcome following surgery. Given these associations, we further investigated whether DDR1^{high} or DDR2^{high} bladder tumors correspond to clinical response toward ICT which is currently being evaluated in the adjuvant clinical setting following surgery. Using the data from the IMvigor210 clinical trial, where bladder cancer patients were treated with Atezolizumab (anti-PD-L1), we applied the methodology above to assess if DDR1 and DDR2 gene expression is associated with immune infiltration using IMvigor210 study data. Consistent with the result of TCGA BC cohort, IMvigor210 data showed the same pattern of correlation between DDR1/2 expression and immune infiltration scores of various immune cell types (FIG. 13A). Unsupervised hierarchical clustering analysis of DDR1 and DDR2 expression using Pearson's coefficient similarity and complete linkage method shows clear separation of DDR1^{high}; DDR2^{low} versus DDR1^{low}; DDR2^{high} tumors (FIG. 13B). Spearman's rank correlation coefficient shows that DDR1 and DDR2 expression have inverse linear relationship (rho=−0.45, p=0.0001) in IMvigor210 (FIG. 13B). We also found that DDR1 and DDR2 gene expression did not correlate with response (FIG. 13C-13F) or stratify overall survival (FIG. 13G, 13H) to ICT. Additional stratified GEP scores by DDR1 and DDR2 gene expression did not improve stratification (FIG. 131, 13J).

TABLE 11
Multiple Log-Rank tests were performed with DDR^{low} &
GEP^{high} group in IMvigor210 cohort as a base line. Table
shows Hazard ratio (HR), significance level (P-value)
and confidence interval (CI) for each comparison.

| Comparison | HR | P-value | CI 95% |
|---|---|---|---|
| DDR1 Hi & GEP Low vs. | 0.58 | 0.033 | 0.35-0.95 |
| DDR1 Low & GEP Low | | | |
| DDR1 Low & GEP Hi vs. | 0.56 | 0.026 | 0.34-0.93 |
| DDR1 Low & GEP Low | | | |
| DDR1 Hi & GEP Hi vs. | 0.64 | 0.107 | 0.37-1.10 |
| DDR1 Low & GEP How | | | |
| DDR2 Low & GEP Low vs. | 1.74 | 0.089 | 0.92-3.28 |
| DDR2 Low & GEP High | | | |
| DDR2 Hi & GEP Low vs. | 2.51 | 0.007 | 1.28-4.91 |
| DDR2 Low & GEP High | | | |
| DDR2 Hi & GEP Hi vs. | 2.02 | 0.028 | 1.08-3.79 |
| DDR2 Low & GEP High | | | |

1.4 Development of DDR-Driven Gene Signatures from Animal Model Data

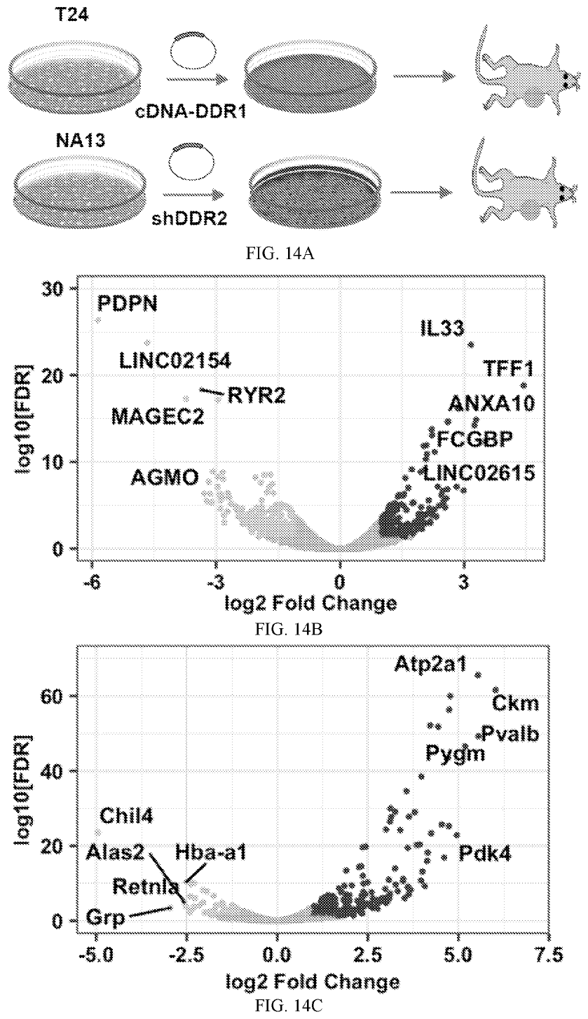

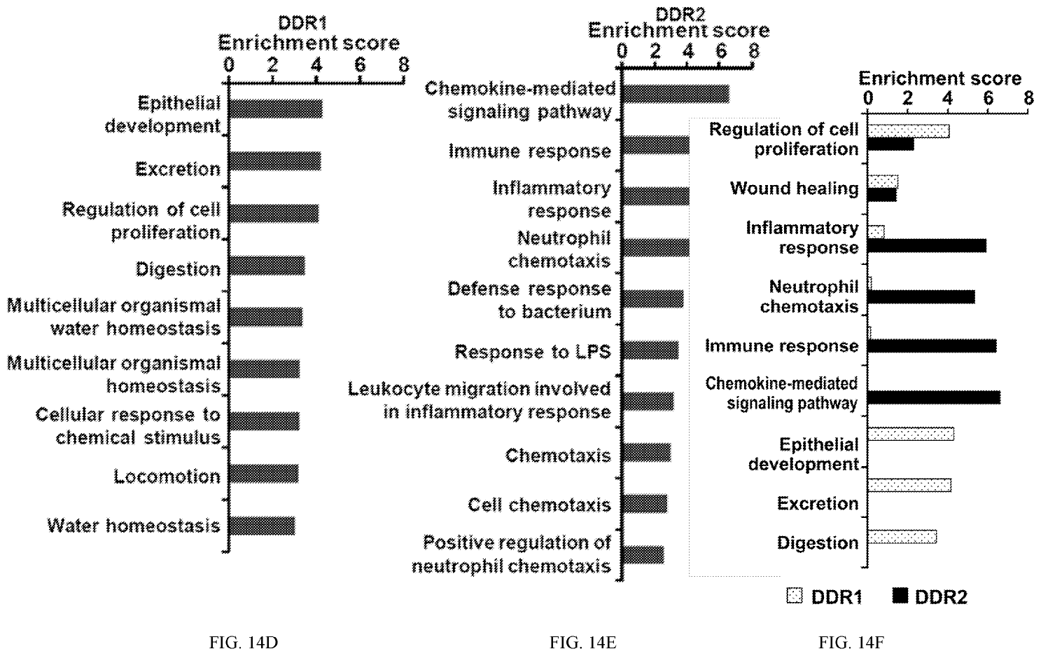

The lack of response or outcome stratification following ICT as a function DDR1 or DDR2 expression or GEP led us to hypothesize that the specific determinants of response to ICT, if present, may consist of a subset of genes that are specifically regulated by DDRs which would not be captured by assessment of DDR1 and DDR2 expression alone. Thus, changes in expression of these genes would represent a biologically active DDR signaling pathway in the tumor compared which DDR expression per se, would not necessarily reflect. This rationale provided the impetus to develop gene-signatures representative of changes in DDR1 and DDR2 status as we have done before for GTPases. To accomplish this, we perturbed DDR1 and DDR2 function via enforced DDR1 expression and DDR2 knock-down, respectively in human and murine tumor models. First, xenografts generated from DDR1 overexpressing T24 human bladder cancer cells and control were subjected to RNA-seq profiling (FIG. 14A). Second, NA13, a recently described murine tumor cell line with high DDR2 expression was transduced with shDDR2 or scrambled shRNA and injected in C57B6 mice to generate tumors which were subsequently subjected to RNA-seq profiling (FIG. 14A). Differential expression analysis was performed in these two models and the corresponding volcano plots were illustrated in FIGS. 14B and 14C. From the DDR1-overexpression model, 225 up-regulated genes and 367 down-regulated genes were identified with FDR<0.05 and |log 2-fold-change|≥1. From the DDR2-shRNA model, 211 up-regulated genes and 69 down-regulated genes were identified with FDR<0.05 and |log 2-fold-change|≥1. Functional enrichment analysis using DAVID software of 225 up-regulated genes by DDR1 overexpression and 69 down-regulated genes by DDR2 knocking down revealed distinct association of cellular processes. DDR1 overexpression is associated with cellular processes (e.g., excretion, extracellular matrix organization, and cell migration) (FIG. 14D), while DDR2 knockdown is associated with cellular processes (e.g., chemokine signaling and immune responses) (FIG. 14E). Intriguingly, regulation of cell proliferation and wound response are two enriched cellular processes that are shared between DDR1 and DDR2 while other processes were clearly different between the two DDRs (FIG. 14F).

The 225 upregulated genes with FDR<0.05 and log 2-fold-change≥1 from the DDR1-overexpression model are: ABO, ASIC1, ADORA2A, ALDH3A1, ANK1, APOC1, AQP3, BDNF, CA12, DDR1, CALB1, CALML3, CBS, CHGA, CHI3L1, CKB, CLN3, COL6A3, ATF6B, CRIP1, CRIP2, CSPG4, CXADR, CYP3A7, CYP2B6, CYP2B7P, AKR1C1, AKR1C2, DEFB1, EEF1A2, MEGF6, EPB41L1, ERG, FGFR3, FOXF2, FOXE1, FMOD, FOSB, GATA2, GDF1, GGT5, GIPR, GLUL, HAS2, ID2, IGFBP2, IL1RN, IL2RB, ITPK1, KCNK2, KRT6A, KRT13, KRT14, LCN2, LGALS3, LSP1, MAOA, MAT1A, MEFV, RNR1, RNR2, MUC1, MUC5AC, MUC6, CEACAM6, CCN3, NPR3, NTSR1, PALM, PAX7, PDE2A, PER1, PLTP, PRKCH, PRODH, PTPRM, RAP1GAP, RPS16, SERPINB3, SCNN1A, SCNN1B, SCNN1G, SLC14A1, SLC15A1, SSTR5, STC1, TBX1, TFF1, TFF3, TGFBI, TGM1, TCHH, TNNI2, TNNT3, SCGB1A1, SLC14A2, CAVIN2, ITGA10, LY6D, KCNK5, FCGBP, SYNGR1, DHRS3, RPS6KA5, SLC9A3R2, TP53I11, FBLN5, NEBL, AGR2, OLFM4, ERN2, NOXA1, ADAM28, ANXA10, NXPH4, SULF1, COBL, TTC9, VSIG2, SPDEF, BACE2, PAMR1, CPNE7, GALNT9, EGFL7, CEND1, BPIFA1, TPPP3, ARHGEF38, PALMD, ENOX1, SLC38A4, ANO1, SYBU, PCDHGB2, ANKH, SUSD2, JPH1, NDUFA4L2, PAK6, AKR1B10, PELI2, SORCS2, VSIR, PDIA2, DBNDD1, MMRN2, TMC5, TMEM254, IQCN, AMN, EPPK1, CCDC3, HMCN1, PPP1R1B, HOPX, AGBL4, TOX2, SHANK3, KIAA1671, SLC25A21, SYT8, MLIP, LINC00473, IL33, CREB3L1, B3GNT7, LOC93429, SYTL5, FOXQ1, EGLN3, CSMD2, TNFRSF13C, PHACTR3, TMEM45B, ADSS1, LYPD6B, C7orf57, SLITRK4, NEURL2, PTGR2, SSTR5-AS1, SLC47A2, SLC16A14, FITM1, CALML6, PLAC4, PLPP4, RANBP3L, HMCN2, CYP4X1, H19, CES4A, C16orf54, MILR1, TMEM145, LURAP1L, ITPK1-AS1, LINC02120, KRT27, ARHGAP40, KRT16P6, GALNT18, IGFL1, CAPN8, C4orf50, CHCHD10, MBNL1-AS1, C9orf152, LINC01667, ANKRD20A3P, RNF165, RBBP4P1, RNF224, C15orf56, SYS1-DBNDD2, RNA5-8SN5, MRPL23-AS1, MIR3911, LINC02582, LINC02615, DNAAF4-CCPG1, PINCR, LOC102724788, and LOC105379194.

The 367 down-regulated genes with FDR<0.05 and |log 2-fold-change|≥1 (i.e., log 2-fold-change≤−1) are from the DDR1-overexpression model are: PDPN, LINC02154, MAGEC2, RYR2, AGMO, LOC101928336, PTPN7, ANO2, RTL1, POTEE, MYH2, CD69, COL14A1, FAM133A, HSD11B1, LOC440910, GABRG3, MAL, LOC339260, MYH1, TNFRSF4, HERC2P4, LOC101927513, ADAMTS9, COLEC10, LINC012 04, LNCAROD, ADAMTS12, MMP9, HNF4G, ACTA1, POTEKP, LINC00839, NUP62, CH25H, LINC01239, ANKRD20A19P, OR13H1, ITGAM, KRT81, PCNPP3, ZNF705D, ROS1, KRT8P8, PTPRD, KCNT2, MYOZ1, DISC1FP1, MIR3142HG, NLRP3, GABRQ, MSR1, ALPK2, LINC02 328, LNCOG, PROM1, STX17-AS1, CLTRN, BMS1P12, ARL14EPL, SLC12A8, PHF2P2, ST8SIA1, MAGEA3, NaN, TINAG, DACH1, CYP39A1, LINC01842, ADAM23, TDRP, FHAD1, OSTN, ZNF197-AS1, CASC9, ILDR2, GABRA3, RAB39B, CSRNP3, PCP4L1, ATP6V0A4, PLAC1, LINC01694, FAR2P2, HS6ST2, THY1, OTOF, WIPF1, SLA, CS MD3, SOX6, GRK3, LPL, LINC01483, RNF182, ZNF229, IL7R, ADAMTS18, LINC01234, SDK 2, C4orf48, PGM5, MANCR, RP1, UBE2V1, MYH16, WT1, SIRPB2, AFF3, LINC00964, PTX3, S LC8A1, FAM96AP2, SIDT1, ADAMTS20, HPX, SCARF1, L3MBTL4, SFRP1, DYNC1I1, SCN 3A, CAPN14, YES1P1, GPC6, IL32, LYPD1, EEF1A1P24, CYP2J2, ITGB6, LOC100506258, CN PY2, NPY1R, SEMA3E, IGFBP7-AS1, CFHR3, MAGEA2, TNIK, APCDD1L-DT, LCP1, TUBAP2, TLR1, SGIP1, CD163L1, CSF2, CADM1, MME, GABRG2, C3orf70, RN7SL674P, EBF1, SMARCA5-AS1, G0S2, CACNA1D, NUP62CL, RABGEF1, OR2W6P, HKDC1, SLC40A1, PCDH17, PROX1, SOGA3, FAR2P1, LINC01358, OR2B2, DOC K2, CACNA2D3, MIG7, LOC102725191, ZBP1, LRP1B, STPG4, GAB3, LINC02389, IFNA1, A CTN2, SLC7A11-AS1, KCNJ16, ACY1, CD83, MMP10, DHRS2, RBM22P2, EGF, MBL1P, CD38, TNFRSF9, LDB2, DAPP1, SCG5, FAM155B, MGAM, LINC00601, FOXP2, VWDE, TBXAS1, TSPEAR, IL1A, PXDN, GRAP, SCN2A, NCAM1, ZEB2-AS1, PDE11A, DMRTA1, PTN, GALNT3, NaN, MGC32805, S1PR3, CSAG3, CTNNA3, TIE1, FEZF1-AS1, FAM172BP, LINC02185, CD200R1, CELF2, LRTM1, LINC02433, PKNOX2, SERPINA6, KRTAP2-3, TNFSF18, HERC5, TUFMP1, ROBO4, FAM89A, CLDN24, TRPM2, CTSS, IL1B, CCN4, AMTN, BIRC3, BATF2, GIMAP5, SEMA6B, SLC2A3, P KDCC, SMPD4P1, FGF5, GDI2P2, PCDHB2, TCIM, GBP5, GPC3, COL5A3, RPSAP36, KIAA1 755, MAGEB17, TEX13B, FTCDNL1, DENND2A, CECR7, PSG4, DNAH6, TTLL11-IT1, NEGR1, DTNA, RASGRP3, NOVA1, DUSP8, BCYRN1, MUC12-AS1, CPSF1P1, LOC100132077, TNF, QPRT, OVOS, INHBA, TRIM34, LINC01134, DOCK8, LYPD8, GRPR, NUTM2E, IGSF11, IL31RA, PLCB4, MEF2C, MCOLN2, RSAD2, KCNMA1, P DGFD, PREX1, FYB2, TLR6, COL6A4P1, LOC101927661, VEPH1, TSPAN18, TMPRSS11D, NELL2, COL8A1, ADA2, CPA4, KIF5C, PDGFRL, USP32P3, LOC101928978, FSIP2, CMPK2, ERVMER34-1, EVA1A, HDAC9, NUS1P2, LINC00607, ZEB2, POU2F2, GPR39, SPAG17, FAP, KRT75, TNFSF13B, ST3GAL5, CD177, RHOB, CCL5, SEL1L3, SNORA81, HA S3, CYSLTR2, ADAM8, RAG1, LPXN, DISC1, RNASE7, SHISA3, MAGEA12, SHANK1, BLID, RBM11, TTLL7, NAV3, NFKBIE, CFH, CCL20, TP53AIP1, CSF1, OASL, ATP8A2, HELZ2, SE MA7A, DCHS1, TLR3, SNORD3B-1, KALRN, USP18, RELN, CAPS, APOD, LRRN4, STAMBPL1, MX1, IGF2, PCDH7, TMEM40, ZFPM2, UGT2B17, ADGRV1, EEF1A1P16, GJB2, F3, VGLL3, PLPP3, FRAS1, and ZDHHC14.

The 69 down-regulated genes with FDR<0.05 and |log 2-fold-change|≥1 (i.e., log 2-fold-change≤−1) from the DDR2-shRNA model are: Grp, Alas2, Hba-a1, Myo15b, Csf3, Hba-a2, I113ra2, Alox15, Alox12, Cxcl5, Ighg1, Tmem63c, Cxcl2, Wdr72, Frmd5, Gucy2c, B3galt2, Gabrp, Pparg, Trim66, Ugt2b34, Haol, Tphl, S100a9, Cxcl3, Sicl7a6, Csf 2, Crisp1, Odaph, Alox12e, Nsun7, Krt8, Gal3st1, Gcnt3, Il1a, Ccl24, Fam78b, S100a2, Pigr, Cldn7, Ccl20, Mmp10, Ghr, Osm, Pglyrp1, Unc13b, Sprr2a1, Car4, Foxp2, Ltf, Muc1, Cxcl1, Fcna, Ppbp, Ef cab3, Krt7, Chn2, Basp1, B3galt5, Vamp1, Misp, Rnfl28, Atg9b, Cda, Adam28, Prss22, Nupr1, Krt19, and S100a8.

The 211 up-regulated genes with FDR<0.05 and log 2-fold-change≥1 from the DDR2-shRNA model are: Ogn, Ggh, 2300002M23Rik, Klk8, Abcg2, Scara3, Prxl2b, Gyg, Prelp, Cilp, Fgf7, Ephx3, Svep1, Jph2, Krtdap, Cacnb1, Mb, Ccn5, Itgb3 bp, Krt10, Lipk, Speg, F xyd1, Zfhx4, Aplnr, Gkn1, Sdr9c7, Ugt8a, Krtl, Ccr4, Fmod, Ptk6, Gm11992, Cd36, Rnase1, Plce1, Rrad, Zfp365, Fabp3, Kcnma1, Reep1, Epdr1, Synpo2, Apoa2, Igf2, Aif1l, Eya4, Sbsn, Adgrf2, Capn 6, Vtn, Hspb6, Rptn, Cdsn, Sync, Olfml1, Cfd, Itih5, Capn3, Endou, Tnni1, Prkag3, Fhl1, Klk9, Prr9, L ypd5, Clec3b, Tmtc1, Rps12-ps16, Acyp2, Adgrg7, Bmper, Pi16, Kctd4, Scx, Alpk2, Dpp4, Aadacl2, Rnf222, Mef2c, Pnpla1, Clec12a, Tlx1, Ret, Ky, Dhdh, Coro6, Gsdma, P2rx5, Hpdl, Comp, Kcnj12, Amot, Svopl, Tmprss11a, Ddit4l, Rragd, Usp13, Rai2, Bcl11a, Cryab, Rnase2b, Hpn, Hmgc s2, Efemp1, Sele, Stac3, Calm4, Synm, Atp1b2, Kcnb1, Apoc1, Crct1, Ttc25, Myoz2, Scn4b, Tnxb, H spb2, Ankrd1, Camk2b, Asb14, Bmp5, S1c4a4, Abcb4, Gucy1b2, Thbs4, Fbxl22, Snord68, Colq, Du sp13, My14, Mt3, Ucp3, Asprv1, Kprp, Fndc5, Myh1, Pgam2, Unc45b, Itgb1bp2, Flnc, Slpi, Cst6, Srl, Myom1, H19, Asb2, Gm94, Actc1, Lrrc2, Ablim2, Tnnt1, Pla2g2f, Lce3f, Tnnc1, Mlf1, Txlnb, Sgcd, Camk2a, Klhl30, Slc38a4, Hrc, Car3, Dsc1, Klk7, Gpd1, Asb11, Atp1a2, Apobec2, Tmod4, Des, Xir p2, Ankrd23, Eno3, Trim72, Cap2, Ttn, Pkia, Cacna1 s, Dusp27, Tnni2, Ppp1r3c, Klhl41, Phkg1, Ryr 1, Tgm3, Cacng1, Neb, Hspb7, Pdlim3, Sh3bgr, Mylpf, Ampd1, Ank1, Ldb3, Mustn1, Myl1, Casq1, Acta1, Nrap, Xirp1, Tnnc2, Cmya5, Actn3, Tnnt3, Pdk4, Pygm, Atp2a1, Pvalb, Ckm, and Myh4.

### 1.5 Characterization of the DDR Gene Signature Scores in Human Tumors

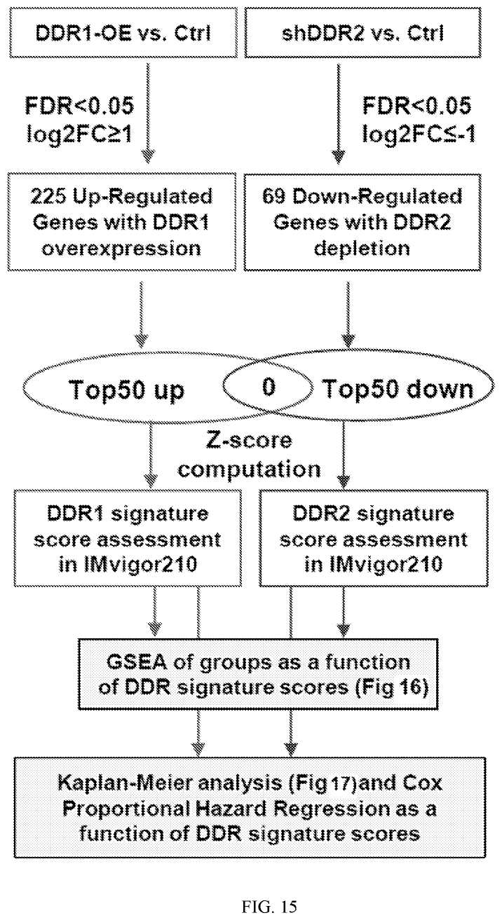

To derive the DDR1 and DDR2 gene signatures, we ranked the 225 up-regulated genes in the DDR1 and 69 down-regulated gene in DDR2 (FIG. 15) in descending and ascending order respectively. We then selected the top 50 most differentially expressed genes in each group (FIG. 15). The top 50 genes were chosen based on recent data from large scale transcriptome analysis which revealed that the top 50 ranked genes in similar comparisons usually provided markers that could discriminate between experimental groups with high confidence. These gene sets had unique composition with no overlap and were employed to derive the gene signatures used to calculate DDR1 and DDR2 scores in human tumors and assess these as a function of molecular, clinical and therapeutic outcomes as presented below (FIG. 15).

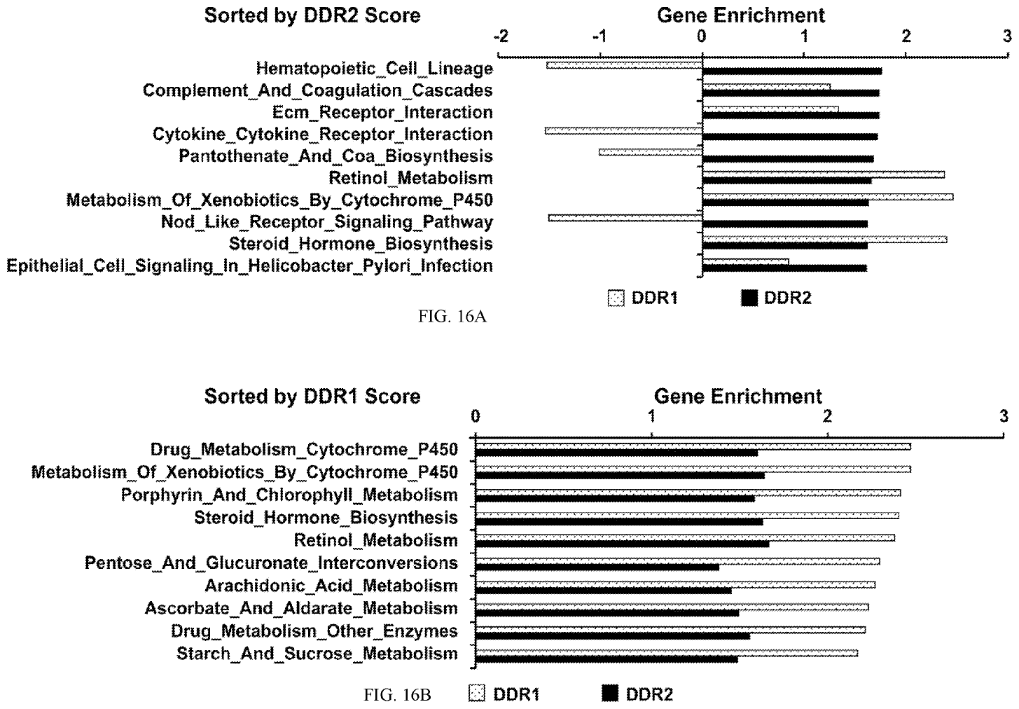

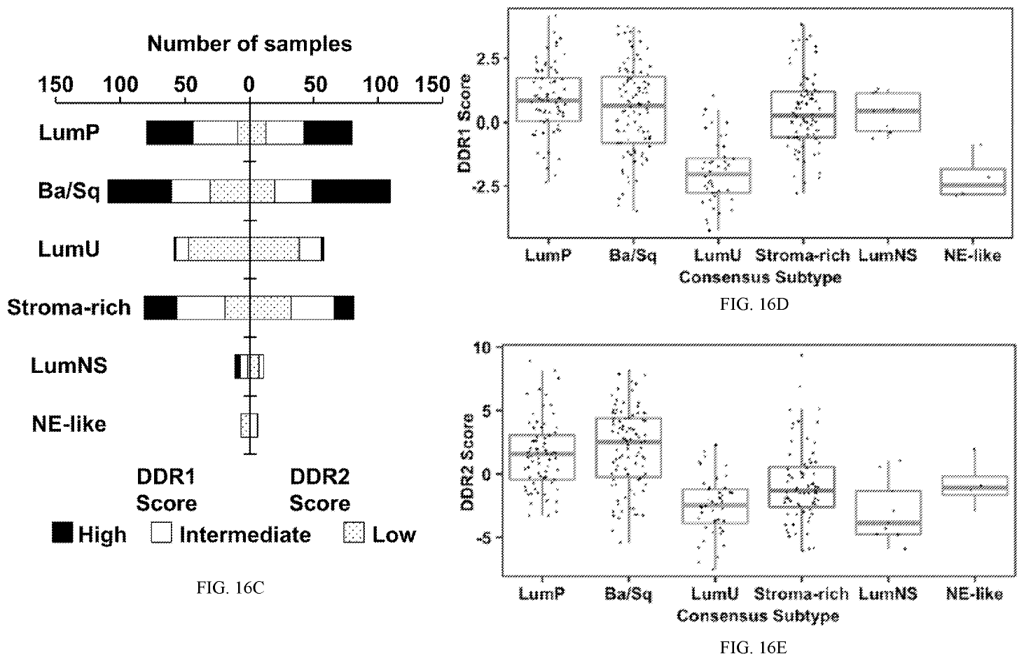

Next we attempted to identify key pathways significantly enriched in bladder tumors with high DDR1 or DDR2 signature scores using GSEA method. To this end, we computed DDR1 score using weighted Z-score method with the top 50 up-regulated genes from the initially identified 225 up-regulated genes from the DDR1-overexpression model; and computed DDR2 score using weighted Z-score method with the top 50 down-regulated genes of the initially identified 69 down-regulated genes from the DDR2-shRNA model (FIG. 15), and stratified IMvigor210 cohort into DDR1^{high} or DDR1^{low} score groups at the median DDR1 score group, and into DDR2^{high} or DDR2^{low} score group at the median DDR2 score. Prior to Z-score calculation, patient gene expression data were centered by median of all the samples. The score represents the difference between the error-weighted mean of the expression values of the genes in the DDR signature and the error-weighted mean of all genes in a sample. GSEA performed for DDR1 and DDR2, separately, resulting that the top10 KEGG pathways are significantly enriched by DDR1 or DDR2 high tumors (FIG. 16A). Consistent with the hallmark gene set enrichment result in FIG. 12A, DDR2 active tumors exhibit significant enrichment of immune related pathways (e.g. Hematopoietic cell lineage, Complement and coagulation cascades, Cytokine-cytokine receptor interaction, and Node like receptor signaling pathway). On the contrary, DDR1 active tumors exhibit significant enrichment of metabolisms (FIG. 16B). Next, we assessed the relationship of consensus subtypes of tumors in the IMvigor210 cohort to DDR1/2 score (FIG. 16C). Of note, LumP and Ba/Sq subtypes have the largest fractions of high DDR1 and DDR2 scoring tumors (FIG. 16A).

1.6 DDR Gene Signature Scores as Predictors of Anti-PD-L1 Response in Patients with Advanced Bladder Cancer

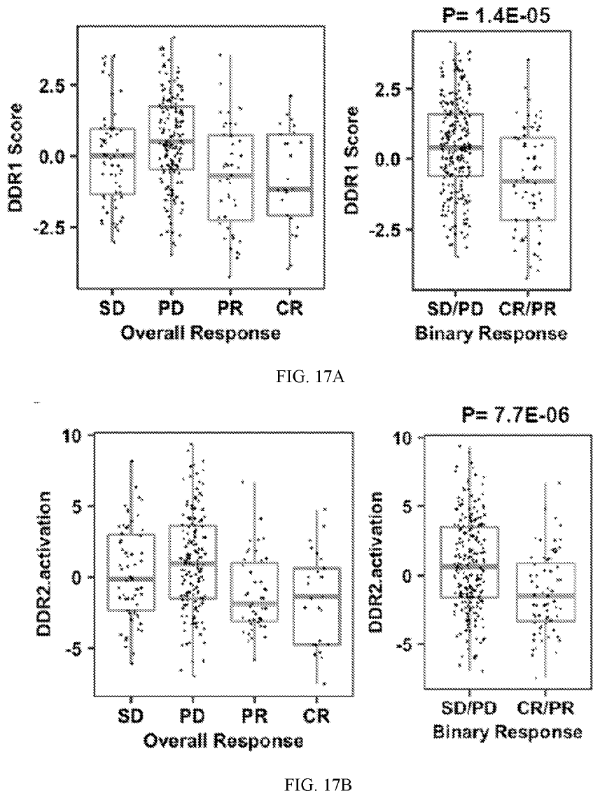

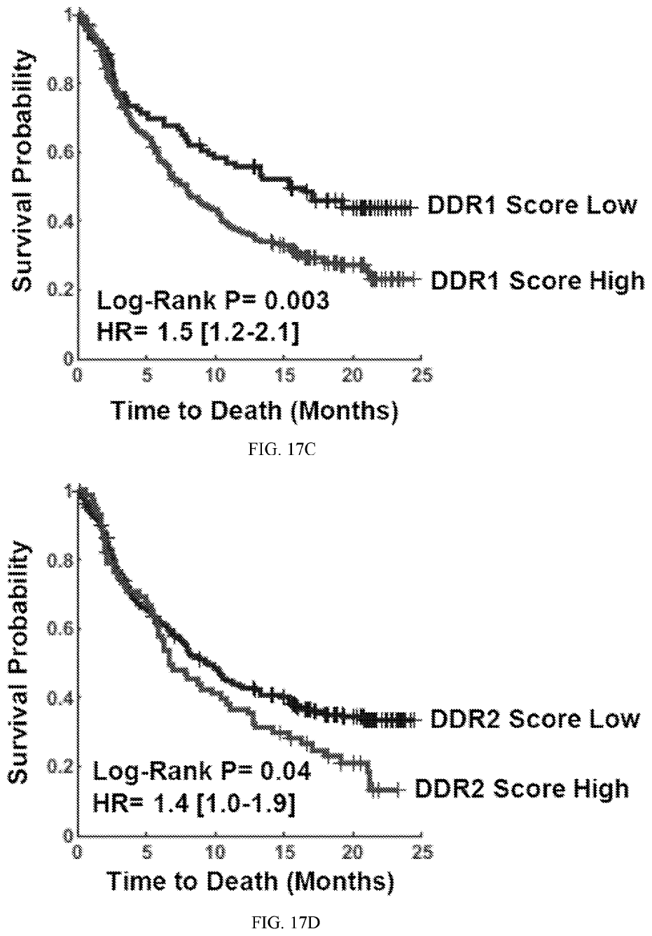

Next, we used the clinical outcome and gene expression data from IMvigor210 to examine the ability of the DDR1 and DDR2 scores to stratify response and survival. We found that DDR scores are significantly different between SD/PD and CR/PR (FIG. 17A, 17B), indicating that DDR scores have the potential to discriminate patient with different responses to anti-PD-L1 therapy. Finally, we tested whether DDR scores are associated with overall survival, which indicated that tumors with high DDR1 (HR=1.5, p=0.003) (FIG. 17C) or DDR2 (HR=1.4, p=0.04) (FIG. 17D) scores have poorer overall survival in IMvigor210 cohort. IMvigor 210 is a phase II trial of atezolizumab (MPDL3280A; an anti-PD-1 monoclonal antibody) in urothelial carcinoma patients who progressed during or following platinum-based chemotherapy.

### 1.7 Independent Validation of Optimized DDR Gene Signatures for Anti-PD-L1 Response Prediction in Lung Cancer

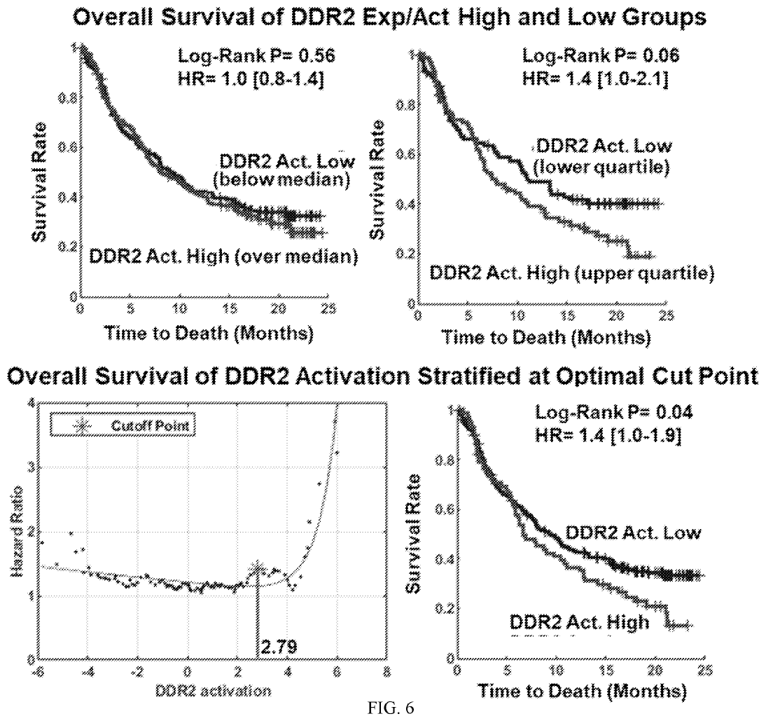

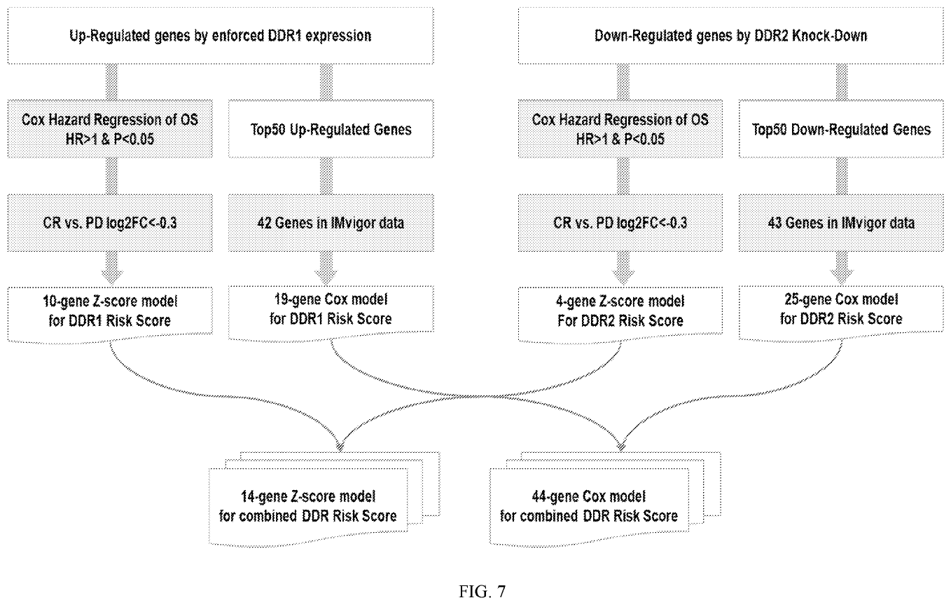

Given the significant correlation of DDR1/2 scores with immunotherapy response and survival in bladder cancer (FIG. 17A-17D), we sought to optimize the composition of the gene signature with the aim to eventually develop it as a rapid and relatively inexpensive clinical biomarker. With this goal in mind, we sought to select core genes among the 50 that are highly correlated with overall survival in IMvigor210, through the two different subtractive approaches (FIG. 7). Finally, we developed four gene signatures: 10-gene signature based on Z-score model (CS-10) and 19-gene signature based on Cox model (CS-19) for DDR1, and 4-gene signature based on Z-score model (CS-4) and 25-gene signature based on Cox model (CS-25) for DDR2 for risk stratification of the patients.

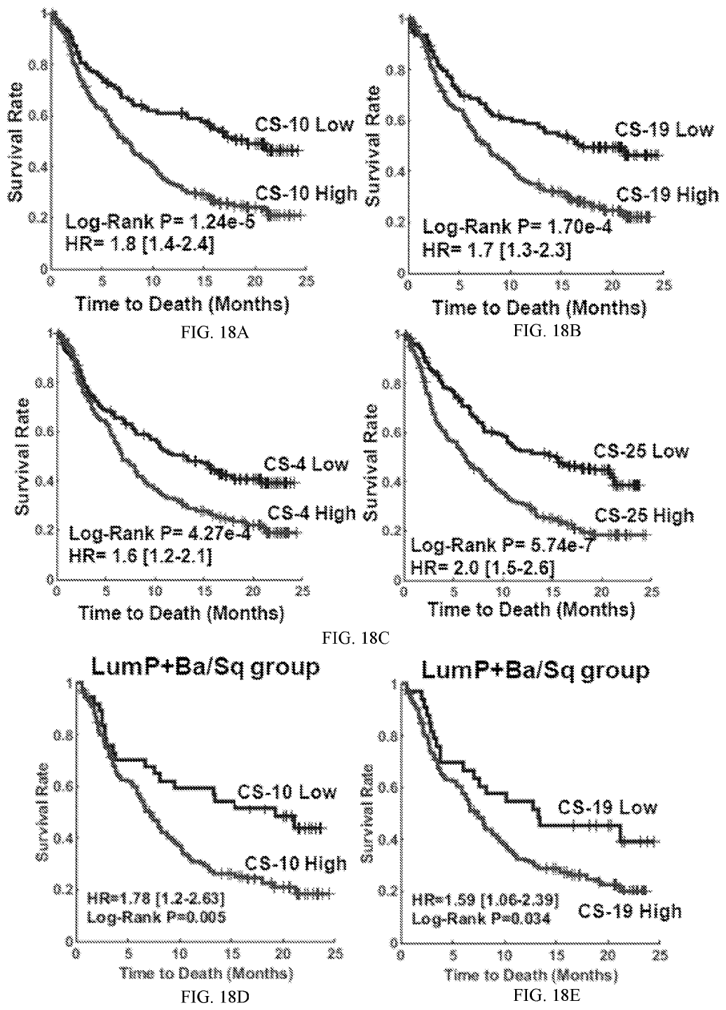

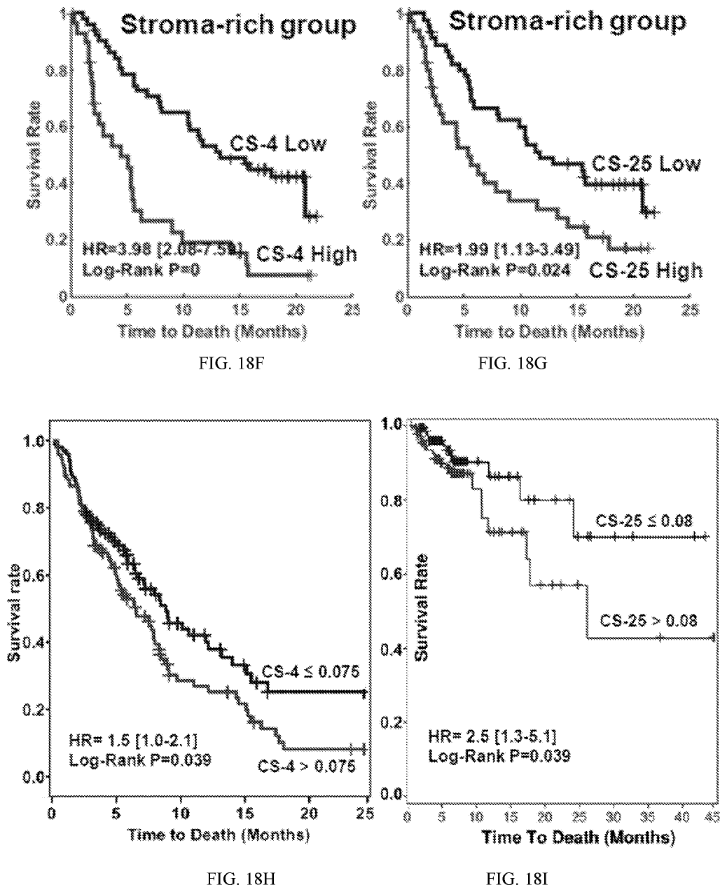

We evaluated the DDR gene signatures by performing Kaplan-Meier survival curve analysis of the IMvigor210 cohort. We first searched an optimal cut-point of signature scores by assessing the trend of HR versus DDR signature scores for overall survival. We found that DDR1 signature scores of −0.77 (CS-10) and −0.079 (CS-19) exhibit the lowest P-values (1.24e-5 and 1.70e-4) and Hazard Ratio (HR)=1.8 and 1.7, respectively, showing the best separation of the two groups (FIG. 18A, 18B). We also evaluated the DDR2 gene signatures using the same way. Optimal cut-point search for DDR2 signature scores were done by assessing the trend of HR versus DDR2 signature scores for OS. We found that DDR2 signature scores of 0.039 (CS-4) and −0.059 (CS-25) exhibit the lowest P-values (4.27e-4 and 5.74e-7) and HR=1.6 and 2.0, respectively (FIG. 18C).

Next, we evaluated whether DDR1 gene signatures (i.e., CS-10 and CS-19) can stratify tumors in the LumP and Ba/Sq subtypes and found that tumors with high score exhibit worse overall survival both with CS-10 (FIG. 18D) and CS-19 (FIG. 18E). We also tested the same hypothesis to the DDR2 and found that in Stroma-rich subtype (FIG. 18F) tumors with high CS-4 (FIG. 18F) or CS-25 (FIG. 18G) scores had worse overall survival. Of note, CS-25 shows the best performance in Stroma-rich subtype and significant stratification of overall survival all subtypes, indicating it could be a molecular subtypes independent predictor of survival post PD-L1 therapy. Here, there are two different score computation methods applied: one is Z-score method, and the other is based on Cox-model parameter weighted sum. The scores of CS-10 and CS-4 are based on Z-score method, and the scores of CS-19 and CS-25 are based on the Cox-model parameters. Once we have the scores (a.k.a. Risk Score or RS), we broke the cohort into two groups: a score high group and a score low group, at the optimal cut-point as described in the immediate prior paragraph. That is, if a sample has a higher score than the optimal cut-point, we assigned it to the high group. So the survival outcome was compared between the score high group and the score low group, and the score low group is control group in all respective the comparisons.

Given the exciting association of DDR1 and DDR2 gene signature scores with outcomes post PD-L1 therapy, we sought to determine if this predictive ability was only applicable to bladder cancer since the gene expression signatures were developed in animal models of bladder cancer, or more widely to other tumor types. Therefore, we identified two independent outcome and molecular datasets from non-small cell lung cancer (NSCLC) patients treated with anti-PD-L1 as a part of routine clinical practice in combination of prior therapy regimen including platinum-based chemotherapy, taxane, tyrosine kinase inhibitor, chemoradiation, VEGF targeted therapy (patient characteristic shown in Table 12).

TABLE 12
NSCLC patient characteristics.

| Characteristic | Tempus | | Caris | |
|---|---|---|---|---|
| No. of patients | 196 | | 259 | |
| Age, Median, years (range) | 67 | (26-90) | 67 | (36-89) |
| Sex, male, n (%) | 95 | (48) | 136 | (52) |
| Race, Caucasian, n (%) | 106 | (54) | not available | |
| Site of primary tumor, n (%) | | | | |
| Lung | 196 | (100) | 259 | (100) |
| ECOG PS, n (%) | | | | |
| 0 | 19 | (10) | not available | |
| 1 | 36 | (18) | not available | |
| Other (2, 3, 4) | 21 | (11) | not available | |
| Unknown | 120 | (61) | not available | |
| Tobacco use, n (%) | | | | |
| Current | 38 | (19) | not available | |
| Never | 30 | (15) | not available | |
| Previous | 110 | (56) | not available | |
| Unknown | 18 | (9) | | |
| Metastatic sites at baseline, n (%) | | | | |
| Visceral* | 131 | (67) | 135 | (52) |
| Liver | 16 | (8) | 18 | (7) |
| Lymph node only | 12 | (6) | 37 | (14) |
| Time from prior chemotherapy ≤3 | 8 | (4) | 31 | (12) |
| months, n (%) | | | | |
| Prior therapy regimen, n (%) | | | | |
| Platinum-based chemotherapy | 88 | (45) | 36 | (14) |
| Taxane | 44 | (22) | 17 | (7) |
| Tyrosine Kinase Inhibitor | 16 | (8) | 4 | (2) |
| Chemoradiation | 33 | (17) | 90 | (35) |
| Targeted therapy** | 37 | (19) | 6 | (2) |
| Pemetrexed | 72 | (37) | 18 | (7) |
| Other | 30 | (15) | 40 | (15) |
| PD-L1 status in tumor-infiltrating | | | | |
| immune cells (Proportion of PD- | | | | |
| L1-stained tumor-infiltrating immune | | | | |
| cells within the tumor area) | | | | |
| ≥5% of the tumor area | 56 | (29) | 129 | (50) |
| <5% of the tumor area | 80 | (41) | 130 | (50) |
| Unknown | 60 | (31) | | |

*Visceral metastases defined as liver, lung, bone, or any nonlymph node or soft tissue metastasis.
**VEGF targeted therapy + TKI.

We then applied our scoring schemes of the four gene signatures to such data. Importantly, this analysis was done in a fully blinded fashion with the DDR score assignments made on the RNA-seq data that was completely devoid of any other information. The survival curves were computed by investigators which had the DDR signature scores for each RNA-seq file but no other information on gene composition or the signature of how the score was derived. It is important to note that the two cohorts have differences in the proportion of PD-L1 stained tumor infiltrating immune cells in the tumor with 29% of patients (n=59) in the Tempus cohort having >5% of PD-L1 positive cells compared to 50% of patients (n=129) in the Canis cohort. This difference may explain why CS-4 exhibited significant separation in the Tempus cohort (Log-rank P=0.039; FIG. 18H), while CS-25 exhibited significant survival separation in the Caris cohort (Log-rank P=0.008; FIG. 18I). Interestingly, both are DDR2 signatures and CS-4 shows higher hazard ratio compared to CS-25 in the Stroma-rich subtype (FIG. 18F, 18G). These suggest that DDR2 gene signatures can predict patient survival for PD-L1 checkpoint inhibitor therapy and use of CS-4 or CS-25 may be guided by the proportion of immune cell infiltration or other clinical or molecular features. Further research will be needed to determine this. It also suggests that DDR2 dependent transcriptional program is linked with immunotherapy response.

### 1.8 Overall

Immune-related biomarkers for stratifying responders from non-responders to ICT can be broadly categorized into two subsets: biomarkers indicative of a T cell-inflamed tumor microenvironment (i.e., PD-L1 expression, tumor infiltrating lymphocytes, and interferon gamma signature), and genomic biomarkers (i.e., tumor mutational burden and mismatch-repair deficiency). The expression of PD-L1, detected through immunohistochemical (IHC) staining of tumor tissue sections, has been associated with clinical response to PD-1/PD-L1 ICT in multiple cancer types such as melanoma, non-small cell lung carcinoma (NSCLC), renal cell carcinoma (RCC), and colon cancer. However, PD-L1 can be expressed on both tumor cells and immune cells, and the predictive value of PD-L1 expression on either tumor or immune cells differs based on the cancer type. For instance, PD-L1 expression on tumor cells is correlated with clinical benefit to ICT in some cancers (i.e., melanoma, NSCLC, and RCC), while epithelial PD-L1 expression in bladder carcinomas do not stratify response. In contrast, PD-L1 expression on immune cells is associated with clinical benefit in both bladder and colon cancer. Additionally, there have been reports indicating technical challenges in interpreting PD-L1 expression by IHC, due to the variability in antibody clones and scoring methods of different PD-L1 IHC detection assays. Perhaps more importantly, other studies have shown that patients with PD-L1-negative tumors can also benefit from ICT, suggesting the presence of biological drivers of response unrelated to PD-L1. Alternatively, tumor biopsy samples on which these assessments are made, may not be representative of PD-L1 expression due to intratumoral heterogeneity. Collectively, PD-L1 expression alone does not seem to represent an ideal predictive marker of response.

Other parameters evaluated as predictors of ICT response include: i) the presence of tumor-infiltrating lymphocytes (TILs), ii) T cell-inflamed gene expression profile (GEP), and iii) interferon gamma (IFN-7) gene signature. In general, high densities of TILs (i.e. CD8+ T cells) have been associated with better clinical outcome after surgical removal of primary tumors. In the context of ICT, targeting PD-1/PD-L1 signaling is thought to reinvigorate pre-existing anti-tumor response of TILs which express immune checkpoint molecules. IFN-γ is a cytokine released by activated T cells, natural killer (NK) cells and NKT cells in the TME and is important for both antitumor response and adaptive immune resistance mechanisms in cancer. In melanoma, ICT responders had higher baseline expression of IFN-γ response genes which lead to expression of cytotoxic molecules (i.e. perforin, granzyme B, TRAIL, and TNF-a), enhancement of antigen presentation machinery, and increased T cell-attracting chemokines important for antitumor response. IFN-γ-signaling also upregulates expression of PD-L1 tumor, stromal and other immune infiltrating cells, which can interact with PD-1 on TILs.

Genomic biomarkers such as tumor mutational burden (TMB) and microsatellite instability (MSI) have also been associated with clinical response to ICT in different cancers. High TMB has been correlated with improved clinical outcome in NSCLC, melanoma, colorectal, and bladder cancer. However, like PD-L1 expression, the variable detection methods, and lack of validated cutoff for TMB detection in bladder cancer limits the predictive capabilities of TMB as a biomarker. Colorectal cancer patient with high MSI due to mismatch repair-deficiency has also shown better clinical response to ICT. The predictive capabilities of TMB and MSI-H status have been attributed to the increased formation of immunogenic neoepitopes presented by tumor cells to immune cells in the context of MHC.

The limitations of predicting ICT treatment response are particularly relevant to bladder cancer which motivated the current study. As is true for other cancers, while PD-L1 expression is not recommended for use in stratifying ICT treatment for all cases, in bladder cancer this is required in specific settings such as patients which cannot tolerate cisplatin or in some cases, any platin based chemotherapy. These limitations of the current predictive biomarkers also highlight the significance of the present study, in which we identified biological-relevant gene signatures connected to a RTK family (i.e., Discoidin Domain Receptor), with its reported functions in mediating immune checkpoint resistance and metastasis in bladder cancer. Such DDR2 and DDR1 scores not only have values in predicting PD-L1 response in bladder cancer but also in non-small cell lung cancer cohorts, indicating a generalization of their putative utilities to other cancer types. Importantly, our findings also make an important link to other recent studies, revealing a role for the stromal microenvironment, extracellular matrix and cognate receptors in the modulation of immune checkpoint resistance in bladder carcinomas. For instance, in the CheckMate 275 bladder cancer patient cohort, a high CD8 T cell infiltration together with a low epithelial mesenchymal transition (EMT)/Stromal core signature was associated with the highest response rates, longest progression-free and overall survival to the anti-PD-1 drug Nivolumab. Conversely, patients with a high CD8 T cell infiltration but also a high EMT/Stromal core signature showed a significantly worse progression-free and overall survival, implicating a role of the stromal bTME in impeding T cell function and thus driving immune checkpoint resistance. We conceive future studies of using the DDR2 and DDR1 scores in predicting immune checkpoint response in the setting of clinical trials of ICT. Furthermore, if such scores are shown to be predictive, it would be important to determine if this translates to superior outcome for patients with high signature scores treated with DDR1/2 inhibitors such as Sitravatinib and Dasatinib in combination with ICT in bladder NCT03606174 and other cancers (lung: NCT02750514, hematologic NCT02819804, NCT02011945).

### 1.9 Materials and Methods

1.9.1 Differential Expression Analysis of RNA-Seq Data from DDR Animal Models

NA13 was a murine C57B6 bladder cancer line and T24 human bladder tumor cells. RNA-seq analysis was done through the standard Illumina RNA-seq protocol. The sequenced reads were put into STAR-RSEM RNA-seq data analysis pipeline to quantify gene level expression. DDR1 data were aligned with Human Genome GRCh38 version, while DDR2 data were aligned with Mouse Genome 19 version. Read counts were normalized by TMM normalization method (Robinson, M. D., et al., Genome Biol., 2010, 11, R25) and Negative Binomial test (Anders, S., et al., Nature Precedings, 2010.4282.1) were applied to identify differentially expressed genes (DEGs) of these comparisons including DDR1 OE versus control (DDR1 in vivo), shDDR2 versus scramble control (DDR2 in vitro), and shDDR2 versus IgG control (DDR2 in vivo). False discovery rate (FDR) was estimated with Storey's method (Storey, J. D., et al., PNAS, 2003, vol. 100, no. 16, 9440-9445). DEGs were selected with FDR<0.05 and log 2-fold-change≥1.

### 1.9.2 TCGA Bladder Cancer Sample Selection

Out of 407 TCGA bladder cancer patient data retrieved on TCGA Bladder Cancer (BLCA) cohort from UCSC Xena browser, we have selected 259 sample data for further analysis. The samples which met the following criteria were excluded from our analysis: 1) stage other than T2-4; 2) variant histology (N=52, 42 squamous, 4 small cell/neuroendocrine, 2 micropapillary, 4 plasmacytoid), pure squamous cell carcinomas (N=3), squamous cell carcinoma of non-bladder origin (N=1) and adenocarcinoma (N=1); 3) prior intravesical immunotherapy with Bacille Calmette-Guerin (N=35); 4) neoadjuvant chemotherapy (NAC) (N=12); and 5) low histological grade (N=21).

### 1.9.3 Estimation of Immune Infiltration Using Cancer Transcriptome

Gene signatures for various immune cells and stromal components were obtained from CIBERSORT, MCP-Counter and xCell. If the genes are overlapped between different immune cell type signatures, only the cell type specific genes were remained. For example, if the B cell subsets such as Memory B cell and Naïve B cell have overlapping genes in the original signatures, we removed the overlapping genes from both signatures to represent specific subset specific expression, resulting signatures have no overlap and 11 and 14 Memory B cell and Naïve B cell signatures, respectively. Finally, we refined gene signatures for 23 immune cell types including Memory B cell, Naïve B cell, Memory CD4 T cell, Activated Memory CD4 T cell, Naïve CD4 T cell, CD8 T cell, Cytotoxic T lymphocytes, Activated Dendritic cells, Eosinophil, M0 Macrophage, M1 Macrophage, M2 Macrophage, Activated Mast cell, Monocyte, Activated NK cell, Neutrophil, Plasma cell, Activated Dysregulated T cell, Dysregulated T cell, Exhausted T cell, Follicular Helper T cell, Gamma Delta T cell, Regulatory T cell. Given these gene sets for immune cell types, we computed immune score using MCP-counter method (Aran, D., et al., Genome Biology, 2017, 18, 220). Briefly, Aran, D. et al. describe that the raw score is the average single-sample GSEA (ssGSEA) score of all signatures corresponding to the cell type, that using simulations of gene expression for each cell type, a function is derived to transform the non-linear association between the scores to a linear scale and the dependencies are derived between cell type scores, and that a spillover compensation method is applied to adjust the scores, which includes using simulations to generate a spillover matrix that allows correcting for correlations between cell types.

### 1.9.4 Computation of DDR Signature Score

Weighted Z-score method (Levine, D. M., Genome Biology, 2006, 7, R93) was employed to compute DDR signature scores in individual tumors with the DDR1 and DDR2 gene signature. Prior to the score calculation, patient gene expression data were centered by median of all the samples. The score represents the difference between the error-weighted mean of the expression values of the genes in the DDR signature and the error-weighted mean of all genes in a sample. The result reflects both the magnitude and relative direction of DDR downstream genes.

### 1.9.5 Differential Expression Analysis of Distinct Response Groups in IMvigor210 Data

Log 2-scaled and TMM normalized expression data were employed to test differential expression of the genes. Differential expression was assessed by Negative Binomial testing method and FDR was estimated by Storey's method.

### 1.9.6 Refinement of DDR Gene Signatures for Predicting Response to PD-L1 Therapy

We start with up-regulated genes from DDR1 RNA-seq data and selected genes satisfied these two criteria: 1) Cox Proportional Hazard Regression of overall survival P-value<0.05 and HR>1 and 2) log 2-fold-change<−0.3, which is 95 percentile cutoffs of differential expression between CR versus PD in IMvigor210 cohort. This yield 10-gene signature (CS-10) that are high correlation with both poor survival outcome and progressive disease. In a different way, we also performed backward feature selection based on Cox regression of OS with 42 genes detected in IMvigor210 data. Comparison of full model with all 42 genes and reduced model with subset of the 42 genes were conducted with log-likelihood test and 19 genes were selected for optimal subset for the DDR1 Cox model (CS-19). Using the same way, we selected 4 genes (CS-4) and 25 genes (CS-25) for Z-score model and Cox model, respectively. Finally, four gene signatures were developed: 10-gene signature based on Z-score model (CS-10) and 19-gene signature based on Cox model (CS-19) for DDR1, and 4-gene signature based on Z-score model (CS-4) and 25-gene signature based on Cox model (CS-25) for DDR2.

### 1.9.7 Statistical Analysis

To examine the association between clinical outcomes and DDR signature scores, we used Kaplan-Meier survival analysis and Cox proportional hazards regression analysis with the following outcomes: overall survival (OS). To test whether DDR signature performance was prognostic independent of other clinical variables, multivariable analyses were performed adjusting for pathological grade. All analyses were conducted using MATLAB (version 2.7) and R (version 3.2.1). P<0.05 was considered statistically significant.

### Example 2. Derivation of Novel Gene Signature for the Stratification of Immune Checkpoint Blockade Response in Cancer

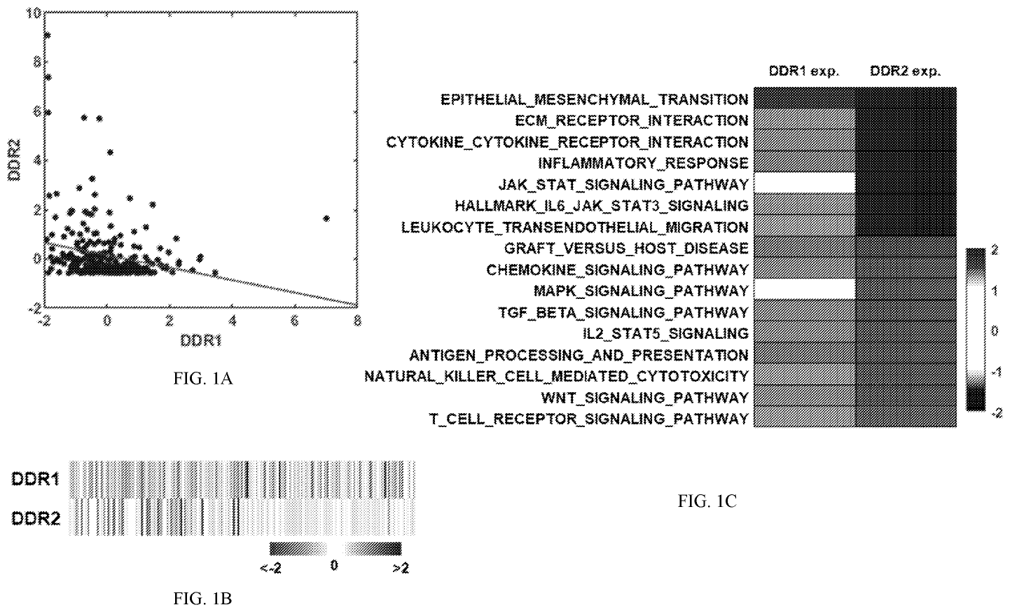

We have assessed DDR1 and DDR2 gene expression in the cancer genome atlas (TCGA) bladder cancer (BC) with TNM stage 2 and higher (n=259). Spearman's rank correlation coefficient shows that DDR1 and DDR2 expression have inverse linear relationship (rho=−0.3, P=0.00001) in the TCGA BC (FIG. 1A). Unsupervised hierarchical clustering analysis of DDR1 and DDR2 expression using Pearson's coefficient similarity and complete linkage method shows clear separation of DDR1-high and DDR2-low versus DDR1-low and DDR2-high tumors (FIG. 1B). These results indicated that based on the DDR1 and DDR2 expression, BC can be divided into two groups with inverse relationship.

We further examined whether cellular processes are differentially represented by the combination of DDR1/DDR2 expressions. To this end, we performed gene set enrichment analysis (GSEA). We first stratified TCGA BC tumors into DDR1 (or DDR2) high and low at the median expression level of DDR1 (or DDR2) and then compared the high and low groups for ranking genes ascending order. Finally, normalized enrichment score of 50 hallmark gene sets were computed for DDR1 and DDR2, separately. Of note, the 16 hallmark gene sets are significantly enriched and genes in the gene sets are highly expressed in DDR2 high tumors compared to DDR2 low tumors, while the genes in these gene sets exhibit low expression in DDR1 high tumors compared to DDR1 low tumors (FIG. 1C). This analysis revealed that DDR1 and DDR2 have inverse relationship in both expression and cellular process levels and 11 out of 16 enriched gene sets in DDR2 high tumors are immune related.

TCGA Exclusion criteria I-VI:

I. any stage other than T2-4.
II. 52 (13%) had urothelial carcinoma with variant histology, including 42 squamous, 4 small cell/neuroendocrine, 2 micropapillary, and 4 plasmacytoid. 5 additional tumors that met screening criteria were excluded: 3 pure squamous cell bladder carcinomas, 1 squamous cell carcinoma of non-bladder origin, and 1 bladder adenocarcinoma. So this total of 57 were excluded.
III. 35 patients had received prior intravesical immunotherapy with Bacillus Calmette-Guerin (BCG) vaccine.
IV. 12 had received neoadjuvant chemotherapy (NAC).
V. 21 low grade tumors.
VI. 3 missing survival information.

### Example 3. DDR1 and DDR2 Signatures as Surrogate Markers for DDR1 and DDR2 Activation

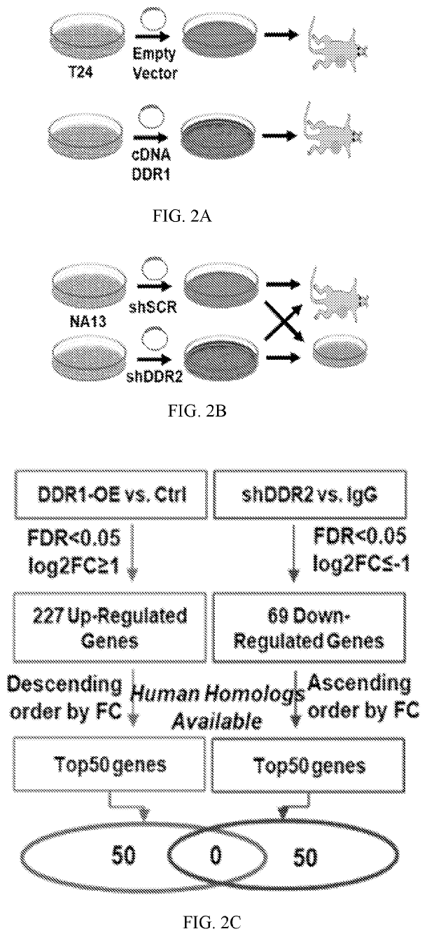

We generated mouse tumors and cell lines models by perturbing DDR1 and DDR2 through enforced DDR1 expression and DDR2 knock-down with shRNA, respectively. DDR1 overexpressing T24 cells, or control (T24 cells carrying empty vector) were injected in the mice and assessed for tumor formation (FIG. 2A); NA13 mouse tumor cells with or without shDDR2 treatment were examined in a cell line model, and also examined after being injected in C57B6 mice, which grew as tumors (FIG. 2B) In a DDR2 experiment, tumors treated with IgG and scrambled shRNA controls (shSCR) were considered as a control group to compare with the shDDR2 treated group. Both cell line and mice tumors were employed to RNA-seq analysis. As a total, there were three pairs of sample groups were transferred to perform RNA-seq analysis, which was done through the standard Illumina RNA-seq protocol. The three pairs were: (1) mouse tumor grown from injected DDR1-overexpressing T24 cells, and mouse tissue grown from injected control T24 cells; (2) mouse tumor grown from injected shDDR2-treated (DDR2 knockdown) NA13 cells, and mouse tissue grown from injected IgG and random scrabble shSCR-treated NA13 cells; and (3) shDDR2-treated (DDR2 knockdown) NA13 cell line, and random scrabble shSCR-treated NA13 cells. The sequenced reads were put into STAR-RSEM RNA-seq data analysis pipeline to quantify gene level expression. DDR1 data were aligned with Human Genome GRCh38 version, while DDR2 data were aligned with Mouse Genome 19 version. Read counts were normalized by TMM normalization method, and Negative Binomial test were applied to identify differentially expressed genes (DEGs) of these comparison including DDR1 overexpression (OE) versus control, denoted as “DDR1 in vivo”); shDDR2 cell line versus scramble control cell line, denoted as “DDR2 in vitro”; and shDDR2 in mice versus IgG control in mice, denoted as “DDR2 in vivo”. False discovery rate (FDR) was estimated with the method described in Storey J D, et al., Proc Natl Acad Sci USA 100, 9440-9445, (2003). DEGs were selected with FDR<0.05 and log 2-fold-change≥1.

From the DDR1 in vivo data, we identified 227 up-regulated genes and 370 down-regulated genes by DDR1 over-expression in T24 tumors. From DDR2 in vivo data, we identified 69 down-regulated genes perturbed by shDDR2. From the DDR2 in vitro data, we have identified 221 up-regulated genes and 349 down-regulated genes by shDDR2. In order to select DDR1 and DDR2 gene expression signature as surrogates for DDR1 and DDR2 activation, we began with 227 up-regulated genes in the DDR1 and 69 down-regulated gene in DDR2 (FIG. 2C). We then sorted the genes with log 2-fold-changes in a descending and an ascending order for DDR1-overexpression related DEGs and for DDR2-knockdown related DEGs, respectively. Finally, we selected the top 50 genes each for DDR1-overexpression related DEGs and DDR2-knowckdown related DEGs to evaluate the performance of these genes as a signature for DDR1 and DDR2 activations in BC.

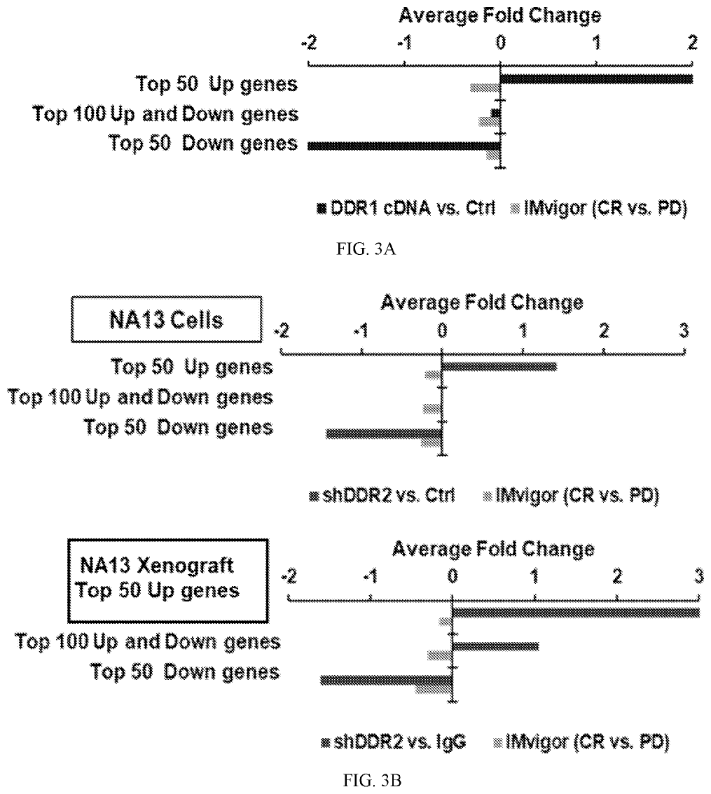

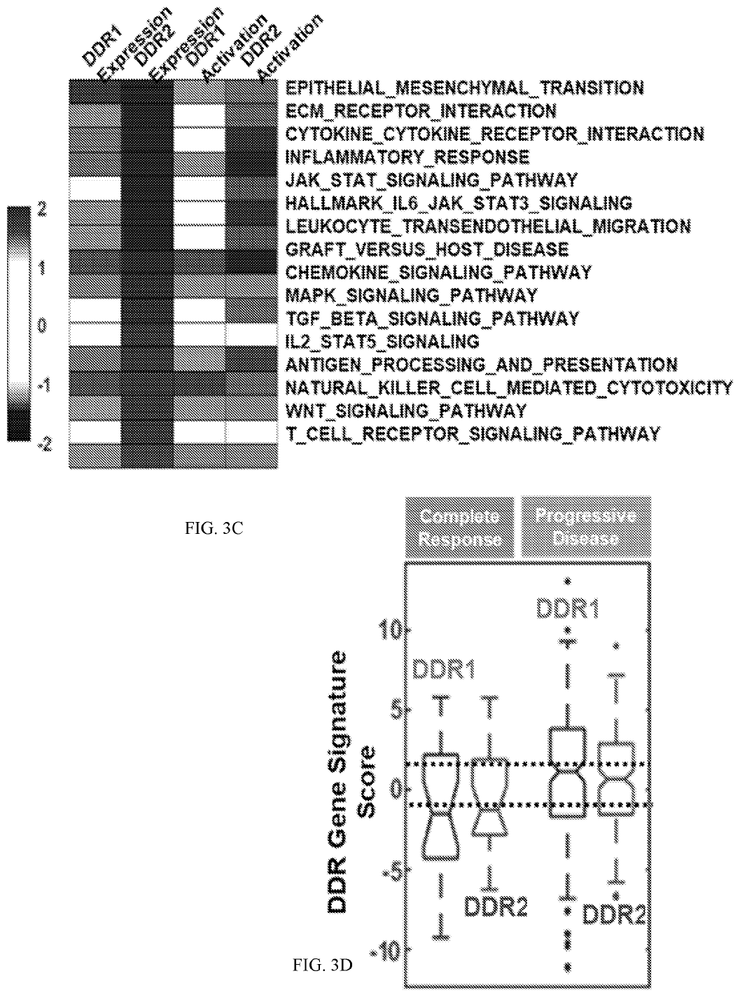

To evaluate the top 50 genes from DDR1-overexpression related DEGs and DDR2-knowckdown related DEGs as representative indicators of DDR1/2 activations in BC, we first computed average fold changes of the distinct combination of the top genes (i.e. top 50 up, top100 up and down, and top 50 down) in DDR and the gene expression data from the IMvigor clinical trial (NCT02108652). This was performed to examine the response to immune checkpoint drug in BC patients and provides comprehensive gene expression data and clinical annotation for the response to the checkpoint drug. We compared expression levels of the top 50 gene sets in the complete responder (CR) and progressive disease (PD) groups from the trial data. We then assessed whether the average fold changes of top 50 gene sets in the DDR data are consistent with IMvigor data (FIGS. 3A and 3B). The average fold change of the top 50 up genes from DDR1 data displayed the largest down-regulation in CR compared to PD. This indicates that the top 50 up genes are down-regulated in CR group and the top 50 gene set has better representation of the down-regulation of DDR1 activation compared to other two combinations (i.e. top100 up and down and top 50 down) (FIG. 3A). We also checked the same combinations in DDR2 and IMvigor data (FIG. 3B). The average fold change of the top 50 down genes from DDR2 in vivo data displayed the largest down-regulation in CR compared to PD, which is consistent that these genes are significantly down-regulated by shDDR2. However, the same combination of top gene sets from DDR2 in vitro data have no difference of average fold changes between the three top gene combinations. It indicates that the top gene set from DDR2 in vivo data has better correlation with IMvigor data. We assessed whether these DDR1/2 activation score based on the top 50 DDR1 and DDR2 genes consistently represent differential enrichment of the 16 hallmark gene sets, which were significantly enriched by DDR1/2 expression difference in IMvigor. We thus computed DDR1 and DDR2 activation score using weighted Z-score method as described in Levine D M, et al., Genome Biol 7, R93, (2006), with the top 50 up- and down-regulated genes, respectively, and stratified IMvigor cohort into DDR1 or DDR2 activation high and low groups at the median DDR activation score. GSEA performed for DDR1 and DDR2, separately, showing that consistent enrichment pattern was observed in the comparison of DDR1/2 activation high versus low groups as shown in the comparison with DDR1/2 expression high and low groups (FIG. 3C). In line with this, we checked DDR1/2 activation score in CR and PD groups, resulting in significantly higher activation of DDR1 and DDR2 in PD compared to CR. It indicates that high activation of DDR1 and/or DDR2 is associated with poor response to the immune checkpoint drug in BC patients.

### Example 4. Clinical Association of DDR1 and DDR2 Activations in TCGA BC Cohort

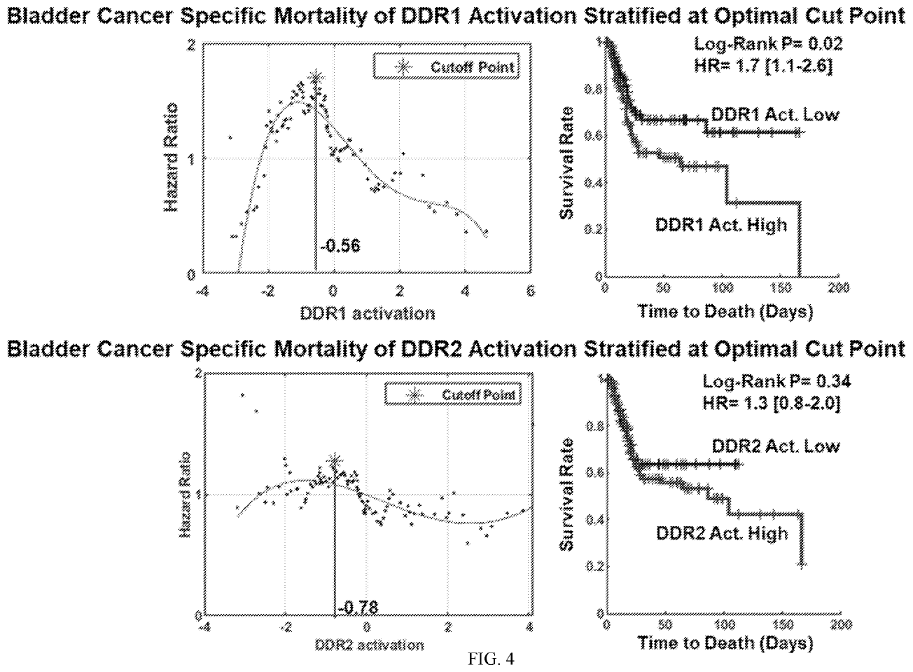

We performed survival analysis to check if the DDR gene activations status are associated with clinical outcomes using TCGA BC data. Bladder cancer specific mortality (BCSM) was employed as a clinical endpoint. We first stratified TCGA BC cohort into DDR1/2 high and low groups to assess the survival association with the BCSM In order to set the optimal point of separation of the cohort, we examined the trend of Hazard Ratio (HR) versus DDR activation scores (FIG. 4 left). Cox proportional hazard regression was evaluated at every cutoff points of DDR activation scores to compute HRs and selected optimal point that have highest HR with the smallest P-value from the Log-Rank test. We found that DDR1 activation at the value of −0.56 showed the highest HR=1.7 and Log-Rank P=0.02 with significant survival difference of DDR1 high and low groups (FIG. 4 upper right), while there is no significant separation point on the DDR2 activation versus HR curve with the highest HR=1.3 and Log-Rank P=0.34, although there is certain level of difference between DDR2 high and low groups after around 50 days (FIG. 4 lower right panels). This indicates that clear association of DDR1 activation and weak association of DDR2 activation with BCSM.

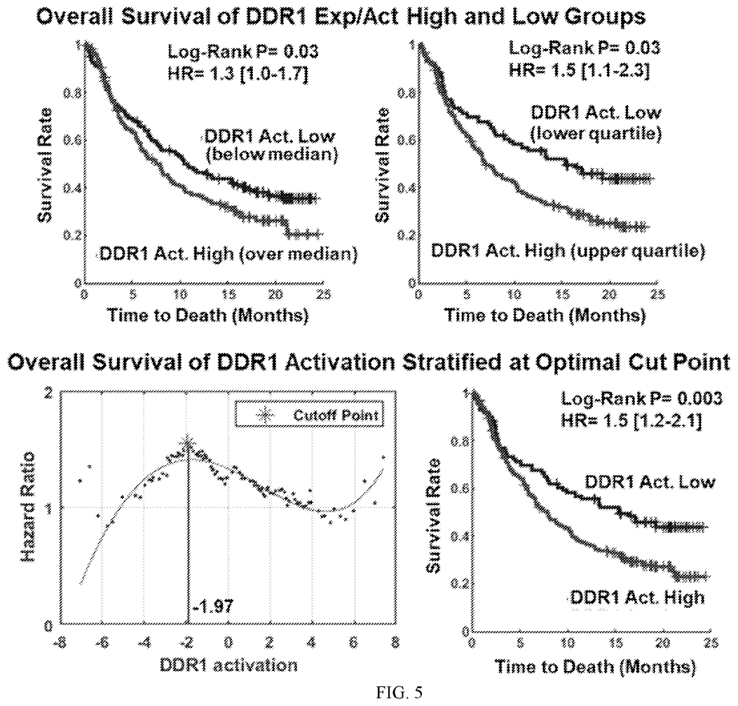

As an independent validation of clinical association with DDR1 activation status in BC, we performed Kaplan-Meier survival curve analysis with three distinct stratifications of the IMvigor cohort. We first checked if cohort division at median DDR1 expression value shows significant separation of overall survival (OS) in IMvigor cohort, indicating significant Log-Rank P=0.03 with HR 1.3 (FIG. 5 upper left). Comparing the two groups with upper and lower quartile of DDR1 activation scores shows larger HR=1.5 with statistical significance (P=0.03) (FIG. 5 upper right). We then searched an optimal cut-point of DDR1 activation by assessing the trend of HR versus DDR1 activation score in IMvigor for OS (FIG. 5 lower left). We found that DDR1 activation value of −1.97 exhibits the lowest P-value (0.003) and HR=1.5, showing the best separation of the two groups (FIG. 5 lower right). It demonstrates that DDR1 activation has significant association with patient survival and may have a link with response to immune checkpoint drug in BC.

We further evaluate the DDR2 activation status by performing Kaplan-Meier survival curve analysis with three distinct stratifications of the IMvigor cohort. The cohort division at median DDR2 expression value doesn't show significant separation of OS rate (FIG. 6 upper left). Comparing the two groups with upper and lower quartile of DDR2 activation scores shows larger HR=1.4 with marginal significance (P=0.06) (FIG. 6 upper right). We also searched an optimal cut-point of DDR2 activation by assessing the trend of HR versus DDR2 activation score in IMvigor for OS (FIG. 6 lower left). We found that DDR2 activation value of 2.79 exhibits the lowest P-value (0.04) and HR=1.4, showing the best separation of the two groups (FIG. 6 lower right). This demonstrates that DDR2 activation has significant association with response to immune checkpoint drug in BC, although it's association with BCSM was not significant in the TCGA.

### Example 5. Gene Panel Selection Process and Validation with Data in IMvigor Study

Given the significant correlation of DDR1/2 transcriptome (indicative of their activation status) with survival outcomes, we attempted to select core genes that are highly correlated with overall survival (OS) through the two different subtractive approaches (FIG. 7). For example in a Z-score model, we started with up-regulated genes from DDR1 RNA-seq data and selected genes satisfying these two criteria: 1) Cox Proportional Hazard Regression of OS: HR>1, P-value<0.05; and 2) log 2FC<−0.3, which is 95 percentile cutoff of differential expression between CR versus PD in IMvigor cohort. This resulted in a 10-gene signature that is in high correlation with both poor survival outcome and progressive disease (Table 3). In a different way, we also performed backward feature selection based on Cox regression of OS with 42 genes detected in IMvigor data. Comparison of full model with all 42 genes and reduced model with subset of the 42 genes were conducted with log-likelihood test, and 19 genes were selected for optimal subset for the DDR1 Cox model (Table 4). Using the same way, we selected 4 genes (Table 5) and 25 genes (Table 6) for Z-score model and Cox model that were downregulated in the DDR2 shRNA model, respectively. Finally, we have integrated two gene models based on the co-expression of these genes from DDR1 and DDR2 models. As a result, we have 14-gene Z-score model (combining Tables 3 and 5) and 44-gene Cox model (combining Tables 4 and 6) for risk stratification of nonresponder BC patients to immune checkpoint blockade.

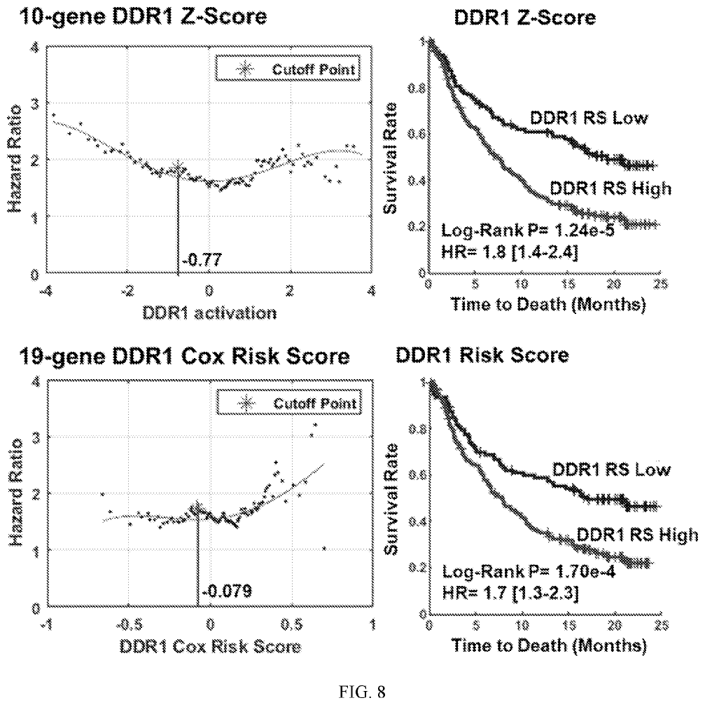

As shown in FIG. 8, we evaluated the DDR1 Risk Scores (RS) by performing Kaplan-Meier survival curve analysis of the IMvigor cohort. This analysis was performed with two gene models (a 10-gene DDR1 Z-score model, and a 19-gene DDR1 Cox Risk Score model). We first searched an optimal cut-point of DDR1 RS by assessing the trend of HR versus DDR1 RS for OS (FIG. 8 left). We found that DDR1 RS values of −0.77 and −0.079 exhibit the lowest P-values (1.24e-5 and 1.70e-4) and HR=1.8 and 1.7, respectively, showing the best separation of the two groups (FIG. 8 right). This demonstrates that DDR1 RSs from the two gene models have significant association with overall survival in BC patients.

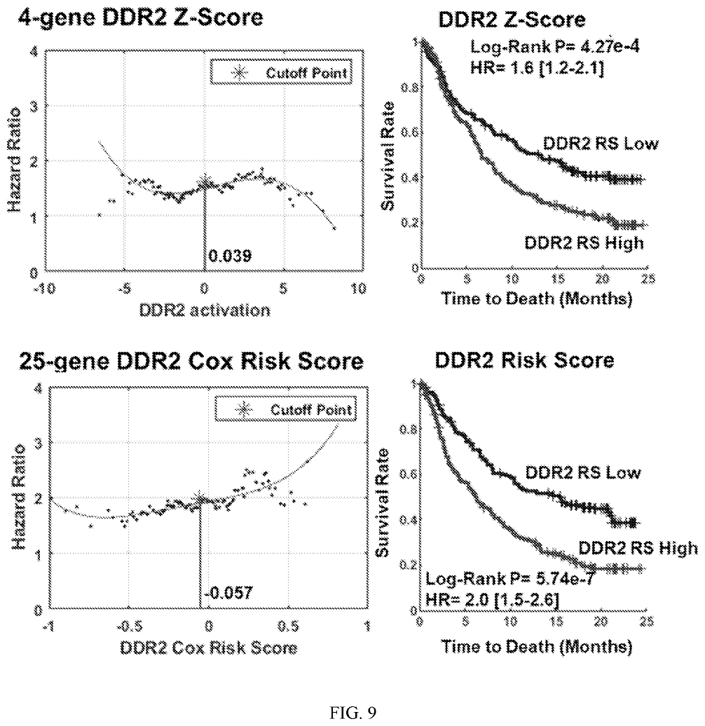

As shown in FIG. 9, we evaluated the DDR2 RS using the same way as did in DDR2 RS evaluation above. This analysis was performed with two gene models (a 4-gene DDR2 Z-score model, and a 25-gene DDR2 Cox Risk Score model). Optimal cut-point search for DDR2 RSs were done by assessing the trend of HR versus DDR2 RS for OS (FIG. 9 left). We found that DDR2 RS values of 0.039 and −0.059 exhibit the lowest P-values (4.27e-4 and 5.74e-7) and HR=1.6 and 2.0, respectively (FIG. 9 right). This also shows that DDR2 RSs from the two independent gene models have significant association with overall survival in BC patients.

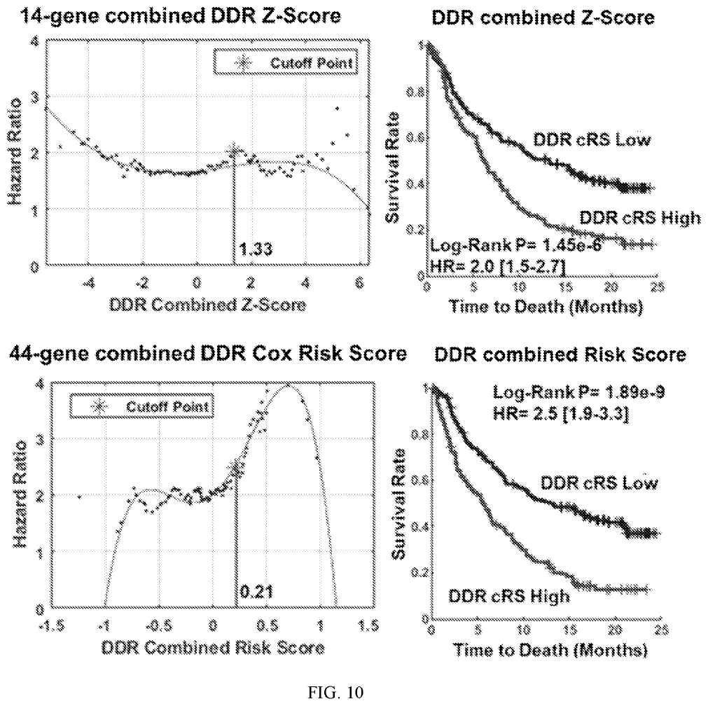

As shown in FIG. 10, we have integrated two combined Risk Score models by unifying the 10-gene and 4-gene models for combined DDR Z-score model and unifying the 19-gene and 25-gene Cox models for combined DDR Cox Risk Model. We evaluated these combined RS (cRS) models using the same way as was done above. Optimal cut-point search for the combined RSs were done by assessing the trend of HR versus cRS for OS (FIG. 10 left). We found that DDR cRS values of 1.33 and 0.21 exhibit the lowest P-values (1.45e-6 and 1.89e-9) and HR=2.0 and 2.5, respectively (FIG. 10 right). cRS models have better correlation with OS compared with DDR1 or DDR2 only models.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A method of selecting a cancer patient for administration of an immune checkpoint inhibitor, comprising:
measuring in a sample of tumor cells from the patient, an expression level of a plurality of marker genes according to claim 16.

2. The method of claim 1, wherein the patient is selected for the administration of an immune checkpoint inhibitor when AQP3, NDUFA4L2, PALM, DHRS3, GGT5, GIPR, GALNT18, ANO1, PCDHGB2, LURAP1L, S100A2, GCNT3, CXCL6, MMP10, TFF1, ANXA10, FCGBP, IL33, TP53I11, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, and CRISP3, if selected, each has an expression level below respective reference value, and NEBL, MLIP, CSMD2, NXPH4, SCNN1B, IGFL1, DEFB1, IL13RA2, ALOX12, TMEM63C, CXCL2, WDR72, GUCY2C, B3GALT2, TRIM66, TPH1, S100A9, od and NSUN7, if selected, each has an expression level above respective reference value, and the patient is not selected for the administration of an immune checkpoint inhibitor when the expression level of AQP3, NDUFA4L2, PALM, DHRS3, GGT5, GIPR, GALNT18, ANO1, PCDHGB2, LURAP1L, S100A2, GCNT3, CXCL6, MMP10, TFF1, ANXA10, FCGBP, IL33, TP53I11, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, or CRISP3, if selected, has an expression level above respective reference value, or when NEBL, MLIP, CSMD2, NXPH4, SCNN1B, IGFL1, DEFB1, IL13RA2, ALOX12, TMEM63C, CXCL2, WDR72, GUCY2C, B3GALT2, TRIM66, TPH1, S100A9, ODAPH, or NSUN7, if selected, has an expression level below respective reference value.

3. The method of claim 1, further comprising obtaining a risk score based on the expression level of the selected plurality of marker genes, wherein the patient is selected for the administration of an immune checkpoint inhibitor when the risk score is below a predefined cut off threshold; and optionally wherein the risk score is calculated by a Z-score metric, or by Cox proportional hazard regression analysis.

4. (canceled)

5. A method of treating a cancer in a subject in need thereof, comprising:
administering an effective amount of an immune checkpoint inhibitor to the subject, wherein the subject's tumor cells have been determined to have expression levels of selected marker genes that are i) a plurality of marker genes having at least 95% sequence identity with genes in Table 3, or homologs or variants thereof, ii) a plurality of marker genes having at least 95% sequence identity with genes in Table 4, or homologs or variants thereof; iii) a plurality of marker genes having at least 95% sequence identity with genes in Table 5, or homologs or variants thereof; iv) a plurality of marker genes having at least 95% sequence identity with genes in Table 6, or homologs or variants thereof, v) polynucleotides which are complementary to the marker genes in any one of i)-vi); or vi) polypeptides encoded by the marker genes of any of i)-vi),
wherein said Table 3 contains genes encoding aquaporin 3 (AQP3), NDUFA4 mitochondrial complex associated like 2 (NDUFA4L2), paralemmin (PALM), dehydrogenase/reductase 3 (DHRS3), gamma-glutamyltransferase 5 (GGT5), gastric inhibitory polypeptide receptor (GIPR), polypeptide N-acetylgalactosaminyltransferase 18 (GALNT18), anoctamin 1 (ANO1), protocadherin gamma subfamily B, 2 (PCDHGB2), and leucine rich adaptor protein 1 like (LURAP1L);
said Table 4 contains genes encoding trefoil factor 1 (TFF1), annexin A10 (ANXA10), Fc fragment of IgG binding protein (FCGBP), interleukin 33 (IL33), nebulette (NEBL), tumor protein p53 inducible protein 11 (TP53I11), transmembrane protein 45B (TMEM45B), muscular LMNA interacting protein (MLIP), ADAM metallopeptidase domain 28 (ADAM28), CUB and Sushi multiple domains 2 (CSMD2), activating transcription factor 6 beta (ATF6B), NDUFA4 mitochondrial complex associated like 2 (NDUFA4L2), neurexophilin 4 (NXPH4), calpain 8 (CAPN8), hemicentin 2 (HMCN2), sodium channel epithelial 1 subunit beta (SCNN1B), aldehyde dehydrogenase 3 family member A1 (ALDH3A1), IGF like family member 1 (IGFL1), and defensin beta 1 (DEFB1);
said Table 5 contains genes encoding S100 calcium binding protein A2 (S100A2), glucosaminyl (N-acetyl) transferase 3 (GCNT3), C-X-C Motif Chemokine Ligand 6 (CXCL6), and matrix metallopeptidase 10 (MMP10);
said Table 6 contains genes encoding gastrin releasing peptide (GRP), aminolevulinic acid synthase 2 (ALAS2), Hemoglobin alpha 2 (HBA2), myosin XVB (MYO15B), hemoglobin subunit alpha 1 (HBA1), interleukin 13 receptor alpha 2 (IL13RA2), arachidonate 15-lipoxygenase (ALOX15), arachidonate 12-lipoxygenase (ALOX12), C-X-C Motif Chemokine Ligand 6 (CXCL6), Transmembrane Protein 63C (TMEM63C), chemokine C-X-C motif ligand 2 (CXCL2), WD repeat domain 72 (WDR72), FERM domain containing 5 (FRMD5), guanylate cyclase 2c (GUCY2C), UDP-Gal: betaGlcNAc beta 1,3-galactosyltransferase polypeptide 2 (B3GALT2), gamma-aminobutyric acid A receptor (GABRP), peroxisome proliferator activated receptor gamma (PPARG), tripartite motif-containing 66 (TRIM66), tryptophan hydroxylase 1 (TPH1), S100 calcium binding protein A9 (S100A9), chemokine C-X-C motif ligand 3 (CXCL3), colony stimulating factor 2 (CSF2), cysteine rich secretory protein 3 (CRISP3), odontogenesis associated phosphoprotein (ODAPH), and NOP2/Sun RNA Methyltransferase Family Member 7 (NSUN7); and
wherein if selected, AQP3, NDUFA4L2, PALM, DHRS3, GGT5, GIPR, GALNT18, ANO1, PCDHGB2, LURAP1L, S100A2, GCNT3, CXCL6, MMP10, TFF1, ANXA10, FCGBP, IL33, TP53I11, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, and CRISP3 each has an expression level below its respective reference value, and if selected, NEBL, MLIP, CSMD2, NXPH4, SCNN1B, IGFL1, DEFB1, IL13RA2, ALOX12, TMEM63C, CXCL2, WDR72, GUCY2C, B3GALT2, TRIM66, TPH1, S100A9, ODAPH, and NSUN7 each has an expression level above its respective reference value.

6. The method of claim 5, further comprising selecting a subject who has been determined to have the expression levels of the selected marker genes in a cancer sample of the subject, before administering the effective amount of the immune checkpoint inhibitor to the subject; or further comprising determining that the subject has the expression levels of the selected marker genes in a cancer sample of the subject, before administering the effective amount of the immune checkpoint inhibitor to the subject.

7. (canceled)

8. The method of claim 5, wherein the cancer comprises bladder cancer, lung cancer, leukemia, or a combination thereof.

9. The method of claim 5, wherein the reference value is an expression level of respective gene from a subject not having the cancer, or from a non-cancerous tissue of the cancer patient or of the subject in need thereof, or wherein the reference value is median level of expression of respective gene from a pool of subjects with the cancer.

10. (canceled)

11. The method of claim 5, wherein the immune checkpoint inhibitor comprises atezolizumab, avelumab, durvalumab, pembrolizumab, nivolumab, cemiplimab, spartalizumab, camrelizumab, sintilimab, tislelizumab, toripalimab, or a combination thereof, or wherein the immune checkpoint inhibitor comprises magrolimab, IBI-188, ALX-148, AO-176, CC-90002, tiragolumab, AB-154, BMS-986207, vibostolimab, BGBA-1217, AB-928, ciforadenant, HTL-1071, PBF-509, EOS-100850, or a combination thereof.

12. (canceled)

13. The method of claim 5, wherein the immune checkpoint inhibitor is administered in combination with an inhibitor of discoidin domain receptor 2 (DDR2), an anticancer drug, an anti-VEGF therapy, chemoradiation, or a combination thereof.

14. The method of claim 5, wherein the plurality of marker genes comprises all the marker genes in any one set of:
a) all marker genes set forth in Table 3;
b) all marker genes set forth in Table 4;
c) all marker genes set forth in Table 5;
d) all marker genes set forth in Table 6;
e) polynucleotides which are complementary to any plurality of the marker genes in any of a)-d); or
f) polypeptides encoded by any plurality of the marker genes in any of a)-d).

15. The method of claim 5, wherein the marker gene or the plurality of marker genes comprises having at least 95% sequence identity with genes set forth in Table 7 or in Table 8.

16. A method of detecting expression levels of a plurality of marker genes in a subject, comprising:
measuring in a biological sample obtained from the subject the expression level of the plurality of marker genes selected from the group consisting of i) a plurality of marker genes having at least 95% sequence identity with genes in Table 3, or homologs or variants thereof; ii) a plurality of marker genes having at least 95% sequence identity with genes in Table 4, or homologs or variants thereof, iii) a plurality of marker genes having at least 95% sequence identity with genes in Table 5, or homologs or variants thereof; iv) a plurality of marker genes having at least 95% sequence identity with genes in Table 6, or homologs or variants thereof; v) polynucleotides which at least in a portion are complementary to the marker genes in any one of i)-vi); vi) polypeptides encoded by the marker genes of any of i)-vi); and vii) fragments of polypeptides of vi).

17. The method of claim 16, wherein the sample comprises cells or tissue from a bladder of the subject.

18. The method of claim 16, wherein the subject is diagnosed with or suspected of having bladder cancer, or wherein the subject desires a prognosis of a cancer following or before receiving an immune checkpoint blockade therapy.

19. A method of determining if a cancer patient is predicted to respond to the administration of an immune checkpoint inhibitor, comprising:
measuring in a sample of tumor cells from the patient a level of expression of a plurality of marker genes according to method 16,
wherein the patient is indicated to be responsive to the administration of an immune checkpoint blockade therapy when if selected, AQP3, NDUFA4L2, PALM, DHRS3, GGT5, GIPR, GALNT18, ANO1, PCDHGB2, LURAP1L, S100A2, GCNT3, CXCL6, MMP10, TFF1, ANXA10, FCGBP, IL33, TP53I11, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, and CRISP3 each has an expression level below its respective reference value, and if selected, NEBL, MLIP, CSMD2, NXPH4, SCNN1B, IGFL1, DEFB1, IL13RA2, ALOX12, TMEM63C, CXCL2, WDR72, GUCY2C, B3GALT2, TRIM66, TPH1, S100A9, ODAPH, and NSUN7 each has an expression level above its respective reference value; or
wherein the patient is indicated to be responsive to the administration of an immune checkpoint blockade therapy when a risk score of the patient based on the expression level of the selected plurality of marker genes is below a predefined cut off threshold.

20. A method of assessing the efficacy or effectiveness of an immune checkpoint inhibitor being administered to a cancer subject, comprising:
comparing the expression level of marker genes measured in a first sample obtained from the subject at a time t0, with the expression level of the marker genes measured in a second sample obtained from the subject at a time t1, said time t1 is subsequent to said time t0,
wherein the marker genes comprise the plurality of marker genes of claim 25, polynucleotides having at least a portion complementary thereto, or polypeptides encoded thereby,
and
wherein the immune checkpoint inhibitor is indicated to be effective in treating the cancer in the subject when if selected, AQP3, NDUFA4L2, PALM, DHRS3, GGT5, GIPR, GALNT18, ANO1, PCDHGB2, LURAP1L, S100A2, GCNT3, CXCL6, MMP10, TFF1, ANXA10, FCGBP, IL33, TP53I11, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, and CRISP3 each has a lower expression level at the time t1 compared to that at the time t0, and if selected, NEBL, MLIP, CSMD2, NXPH4, SCNN1B, IGFL1, DEFB1, IL13RA2, ALOX12, TMEM63C, CXCL2, WDR72, GUCY2C, B3GALT2, TRIM66, TPH1, S100A9, ODAPH, and NSUN7 each has a higher expression level at the time t1 compared to that at the time t0; or
wherein the immune checkpoint inhibitor is indicated to be effective in treating the cancer in the subject when a risk score of the patient based on the expression level of the selected plurality of marker genes at the time t1 is lower than that at the time t0.

21. The method of claim 20, wherein the time t0 is before the immune checkpoint inhibitor is administered to the subject, and the time t1 is after the immune checkpoint inhibitor is administered to the subject.

22. A method of identifying an agent for cancer treatment, comprising:
contacting a molecule of interest with cells or tissues derived from a cancer tissue,
measuring the expression level of a population of marker genes in the cells or tissues in the presence of the molecule of interest, and
measuring the expression level of the plurality of marker genes in the cells or tissues before the contact with or in the absence of the molecule of interest,
wherein the population of marker genes comprises the plurality of marker genes of claim 25, polynucleotides having at least a portion complementary to the plurality of marker genes of claim 25, polypeptides encoded by the plurality of marker genes of claim 25, or polypeptides having at least 95% sequence identity with the polypeptides encoded by the plurality of marker genes of claim 25,
and wherein a decrease in the expression level of AQP3, NDUFA4L2, PALM, DHRS3, GGT5, GIPR, GALNT18, ANO1, PCDHGB2, LURAP1L, S100A2, GCNT3, CXCL6, MMP10, TFF1, ANXA10, FCGBP, IL33, TP53I11, TMEM45B, ADAM28, ATF6B, NDUFA4L2, CAPN8, HMCN2, ALDH3A1, GRP, ALAS2, HBA2, MYO15B, HBA1, ALOX15, CXCL6, FRMD5, GABRP, PPARG, CXCL3, CSF2, and CRISP3, if selected, in the presence of the molecule of interest compared to that before the contact with or in the absence of the molecule of interest indicates that the molecule is an agent effective for treating bladder cancer, lung cancer, leukemia or a combination thereof and/or improving a subject's responsiveness to an immune checkpoint inhibitor in the treatment of the cancer.

23. (canceled)

24. An assay system, comprising:
an assay surface comprising a chip, array, fluidity card, micro-well plate, or a combination thereof; and
nucleic acid probes that comprise complementary nucleic acid sequences to at least 10 to 50 nucleic acid sequences of the plurality of marker genes of claim 25,
and/or antibodies or antigen-binding fragments of antibodies that target polypeptides encoded by the plurality of the marker genes of claim 25.

25. A plurality of marker genes selected from
i) a plurality of marker genes having 95% sequence identity with those set forth in Table 3, or homologs or variants thereof;
ii) a plurality of marker genes having 95% sequence identity with those set forth in Table 4, or homologs or variants thereof;
iii) a plurality of marker genes having 95% sequence identity with those set forth in Table 5, or homologs or variants thereof; or
iv) a plurality of marker genes having 95% sequence identity with those set forth in Table 6, or homologs or variants thereof.
